(19) United States
(12) Patent Application Publication
(10) Pub. No.: US 2023/0098002 A1
(43) Pub. Date: Mar. 30, 2023

(54) MEANS AND METHODS FOR PREPARING ENGINEERED TARGET PROTEINS BY GENETIC CODE EXPANSION IN A TARGET PROTEIN-SELECTIVE MANNER

(71) Applicant: EUROPEAN MOLECULAR BIOLOGY LABORATORY, Heidelberg (DE)

(72) Inventors: Edward Anton LEMKE, Mainz (DE); Christopher Dieter REINKEMEIER, Heidelberg (DE); Gemma ESTRADA GIRONA, London (GB)

(21) Appl. No.: 17/426,338

(22) PCT Filed: Feb. 14, 2020
(86) PCT No.: PCT/EP2020/053883

§ 371 Date: Jul. 28, 2021

(30) Foreign Application Priority Data
Feb. 14, 2019 (EP) 19157257.7

(51) Int. Cl. C12P 21/02; C12N 9/00

(52) U.S. Cl. CPC C12P 21/02; C12N 9/93; C07K 2319/05; C07K 2319/735

(57) ABSTRACT

The present invention is concerned with orthogonal translation systems which allow for the site-specific introduction of non-canonical amino acid residues into a target protein (POI) in a POI-mRNA-selective manner. Specifically, the present invention relates to assembler fusion proteins which bring an RNA-targeting polypeptide (RNA-TP) segment and an orthogonal aminoacyl tRNA synthetase (O-RS) segment into spatial proximity of one another, either by direct linkage in RNA-TP/O-RS fusion proteins, or though the action of “assemblers” fused to each of these segments in assembler fusion proteins (AFPs). The invention also relates to AFP combinations and nucleic acid molecules comprising a POI-encoding sequence together with a targeting nucleotide sequence that is able to interact with an RNA-TP. The invention further relates to nucleic acid molecules, expression cassettes and expression vectors encoding said RNA-TP/O-RS fusion proteins or AFPs, cells comprising same, as well as methods and kits for translationally preparing POIs.

### CROSS-REFERENCE TO RELATED APPLICATION(S)

This application claims the benefit of priority of EP No. 19157257.7, filed Feb. 14, 2019.

### SEQUENCE LISTING

The instant application contains a Sequence Listing which has been submitted electronically in ASCII format and is hereby incorporated by reference in its entirety. Said ASCII copy, created on Feb. 7, 2022, is named 05710_049US1_SL.txt and is 5,207,732 bytes in size.

### FIELD OF THE INVENTION

The present invention is concerned with orthogonal translation systems which allow for the site-specific introduction of non-canonical amino acid (ncAA) residues into a polypeptide of interest (POI) in a POI-mRNA-selective manner. Specifically, the present invention relates to fusion proteins which bring an RNA-targeting polypeptide (RNA-TP) segment and an orthogonal aminoacyl tRNA synthetase (O-RS) segment into spatial proximity of one another. This is achieved by combining an RNA-TP segment and an O-RS segment in one and the same fusion protein (RNA-TP/O-RS fusion protein), or by the action of one or more polypeptide segments which act as “assemblers” (APs) and facilitate a local enrichment of assembler fusion proteins (AFPs) comprising the one or more APs together with an RNA-TP segment or an O-RS segment, thus bringing said RNA-TP and O-RS segments into close proximity of one another. The invention also relates to AFP combinations and nucleic acid molecules comprising a POI-encoding sequence together with a targeting nucleotide sequence (TN) that is able to interact with an RNA-TP. The invention further relates to nucleic acid molecules, expression cassettes and expression vectors encoding said RNA-TP/O-RS fusion proteins or AFPs, cells comprising same, as well as methods and kits for translationally preparing POIs.

### BACKGROUND OF THE INVENTION

The ability to engineer orthogonal (i.e. non-crossreactive) translation systems site-specifically into living cells enables the introduction of new functionality into proteins. However, this is a herculean task, as translation is a complex multistep process in which at least 20 different aminoacylated tRNAs, their cognate aminoacyl tRNA synthetases (RS), ribosomes and diverse other factors work in concert to synthesize a polypeptide chain from the RNA transcript. An ideal orthogonal system would show no cross-reactivity with factors of the host machinery, minimizing its impact on the housekeeping translational activity and normal physiology of the cell.

Towards this goal, genetic code expansion (GCE) is a method that enables reprogramming of a specific codon. With GCE, an orthogonal (suppressor) RS (O-RS) can aminoacylate its cognate suppressor tRNA with non-canonical amino acids (ncAAs). These ncAAs are typically custom designed and harbor chemical functionalities that can, for example, enable protein function to be photocontrolled, encode posttranslational modifications or allow the introduction of fluorescent labels for microscopy studies using click chemistry. To introduce ncAAs site-specifically into a polypeptide of interest (POI), the anticodon loop of the tRNA is chosen to decode and thus suppress one of the stop codons (see, e.g., Liu et al., Annu Rev Biochem 2010, 79:413-444; Lemke, ChemBioChem 2014, 15:1691-1694; Chin, Nature 2017, 550; 53-60). To minimize the impact on the host cell machinery, the Amber stop codon (corresponding tRNA_{CUA}) is often utilized, owing to its particularly low abundance in E. coli, to terminate endogenous proteins (<10%). Nevertheless, in principle any Amber codon in the genome can be suppressed, potentially leading to unwanted background suppression of non-targeted host proteins. If ncAA-modified proteins are recombinantly produced for in vitro applications, this background incorporation might be tolerable as long as the yields of purified full-length protein are acceptable. However, the challenge is different if the host is considered more than just a bioreactor vessel that can be sacrificed for its protein. In order to study the function of a host-cell POI in situ, the physiological condition of that host cell is an important factor. In that context, minimization of background incorporation of the ncAA is particularly required to ensure well-controlled experiments.

At least three elegant approaches have been developed to enable orthogonal translation in E. coli, that is, to decode a specific codon only for the RNA of the POI and not the entire genome. i) Orthogonal ribosomes recognizing a unique Shine-Dalgarno sequence have been developed to decode quadruplet codons, which are then used instead of stop codons to site-specifically encode an ncAA into a POI. (See, e.g., Heumann et al., Nature 2010, 464:441:444; Orelle et al., Nature 2015, 524:119-124; Fried et al., Angew Chem 2015, 54:12791-12794.) ii) Recently, genome engineering has advanced to the stage that E. coli strains can be depleted of selected native codons, providing a genetically clean (e.g. Amber codon free) host background for selective decoding of specific codons only in the POI. (See, e.g., Isaacs et al., Science 2011, 333:348-353; Lajoie et al., Science 2013, 342:357-360; Ostrov et al., Science 2016, 353:819-822; Wang et al., Nature 2016, 539:59-64.) iii) Unique non-canonical codons have been designed using an artificial base pair encoded only in the coding sequence of the POI. This lowers the risk of nonspecific decoding in other parts of the genome (see Zhang et al., Nature 2017, 551:644-647). However, due to genome complexity, it is not straightforward to transfer these orthogonal translation approaches to eukaryotes (see, e.g., Thompson et al., ACS Chem Biol 2018, 13:313-325), in which additionally the Amber codon is highly abundant (20% in mammalian cells).

There is therefore a high demand for strategies for POI-selective orthogonal translation which are versatile and work not only for well-characterized prokaryotes such as E. coli, which are relatively easy to handle and manipulate, but are also applicable to eukaryotic cells. It was therefore an object of the present invention to address this challenge.

### SUMMARY OF THE INVENTION

The inventors found that orthogonal translation systems (OT systems) which are able to selectively translate the mRNA of a POI can be created by generating spatial proximity between the mRNA of the POI and the O-RSs which allow for translationally introducing the ncAA residues into the growing polypeptide chain of the POI. The inventors demonstrated for a variety of POIs, including membrane proteins, that their OT systems allow for site-specifically introducing ncAA residues into a POI in a mammalian cell with selectivity for the mRNA of the POI compared to other mRNAs in the cytoplasm that contain the same stop codon (that is used as selector codon for encoding the ncAA residue of the POI).

In the orthogonal translation systems of the invention, the spatial proximity is achieved by including a targeting sequence (TN) in the mRNA of the POI that can selectively interact with an RNA-targeting polypeptide (RNA-TP), and linking the O-RS with such RNA-TP. Said linkage can be in a fusion protein comprising both, the O-RS and the RNA-TP (RNA-TP/O-RS fusion protein).

In another approach, this can be achieved by the action of one or more polypeptide segments which act as “assemblers” (APs) in facilitating a local enrichment of at least two assembler fusion proteins (AFPs) at least one of which comprising the one or more APs and an RNA-TP segment and at least one other AFP comprising the one or more APs and an O-RS segment, thus bringing said RNA-TP and O-RS segments (RNA-TP and O-RS also designated “effector” or “EP”) into close proximity of one another. The local enrichment of the AFPs allows for the formation of assemblies (OT assemblies, also termed “OT organelles” herein) which can act as artificial orthogonally translating organelles.

The inventors demonstrated that different types of APs can be used. A first type includes APs which drive local enrichment at (previously existing) intracellular structures (such as, e.g., microtubules or the cytoplasmic side of membranes such as the cell membrane or the nuclear membrane, the ER, mitochondrial or Golgi organelles), termed intracellular targeting polypeptide (IC-TP) segments. A second type of APs generates high local AFP concentrations by self-association in the cytoplasm (in particular by phase separation) termed phase separation polypeptide (PSP) segments herein. Said AP types may also be combined with other polypeptide elements having the ability to form multimeric structures, like in particular, coiled coil heterodimers, as formed by synthetic SYNZIP polypeptide pairs. Similarly, said EP types may also be combined with other polypeptide elements having the ability to form multimeric structures, like in particular, coiled coil heterodimers, as formed by synthetic SYNZIP polypeptide pairs. Such multimer formation further improves local enrichment of AFPs.

The inventors further found that AFPs combining different AP types are particularly useful.

In still another approach, AFPs are provided encompassing in a single polypeptide, i.e. fused together, both types of EP segments, i.e. the RNA-TP and O-RS segment, one or both types of AP segments, i.e. the IC-TP and/or PSP segment, optionally supplemented by said polypeptide elements having the ability to form multimeric structures (SYNZIP polypeptide). This provides the advantage that all the elements required for generating an OT system of the invention are included in one single AFP.

Thus, in a first aspect, the present invention relates to an assembler fusion protein (AFP) comprising:
(a) at least one first polypeptide segment acting as assembler (AP) that is selected from:
   (a1) a polypeptide segment derived from an intracellular targeting polypeptide (IC-TP segment), wherein said intracellular targeting polypeptide targets, and thus becomes locally enriched at, an intracellular structural element within or directly adjacent to the cytoplasm; and
   (a2) a polypeptide segment derived from a phase separation polypeptide (PSP segment), wherein said phase separation polypeptide has the ability to undergo self-association in the cytoplasm of a cell so as to create sites of high local concentration in the cytoplasm, and
(b) at least one second polypeptide segment acting as an effector (EP) that is selected from:
   b1) an RNA-targeting polypeptide (RNA-TP) segment, and
   b2) an orthogonal aminoacyl tRNA synthetase (O-RS) segment;
wherein said polypeptide segments are functionally linked in said AFP.

In a second aspect, the present invention relates to an assembler fusion protein (AFP) combination comprising at least two AFPs of the present invention as described herein. Preferably, the AFP combination comprises at least one AFP comprising a RNA-TP segment and at least one AFP comprising an O-RS segment. Including into at least one AFP of said combination a first SYNZIP element and including in at least another AFP of said combination a second SYNZIP element, wherein said first and said second SYNZIP act together by forming a heterodimer structure, represents another advantageous form of said second aspect.

In a third aspect, the present invention relates to a fusion protein (RNA-TP/O-RS fusion protein) comprising:
(i) at least one RNA-targeting polypeptide (RNA-TP) segment; and
(ii) at least one orthogonal aminoacyl tRNA synthetase (O-RS) segment,

wherein said polypeptide segments are functionally linked in said RNA-TP/O-RS fusion protein.

In a further aspect, the present invention provides a nucleic acid molecule, or a combination of two or more nucleic acid molecules, comprising:
(i) a nucleotide sequence that encodes at least one RNA-TP/O-RS fusion protein of the present invention as described herein, or
(ii) a nucleic acid sequence complementary to (i), or
(iii) both of (i) and (ii).

In a further aspect, the present invention provides a nucleic acid molecule, or a combination of two or more nucleic acid molecules, comprising:
(i) a nucleotide sequence that encodes at least one AFP of the present invention as described herein, or
(ii) a nucleic acid sequence complementary to (i), or
(iii) both of (i) and (ii).

In a further aspect, the present invention provides a nucleic acid molecule, or a combination of two or more nucleic acid molecules, comprising:
(i) a nucleotide sequence that encodes at least one AFP combination of the present invention as described herein, or
(ii) a nucleic acid sequence complementary to (i), or
(iii) both of (i) and (ii).

In further aspects, the present invention provides an expression cassette comprising the nucleotide sequence of the nucleic acid molecule, or the combination of nucleic acid molecules, of the present invention as described herein.

In particular embodiments, the present invention provides an expression cassette comprising:
(i) a nucleotide sequence that encodes at least one RNA-TP/O-RS fusion protein of the present invention as described herein, or
(ii) a nucleic acid sequence complementary to (i), or
(iii) both of (i) and (ii).

In further particular embodiments, the present invention provides an expression cassette comprising:
(i) a nucleotide sequence that encodes at least one AFP of the present invention as described herein, or
(ii) a nucleic acid sequence complementary to (i), or
(iii) both of (i) and (ii).

In further particular embodiments, the present invention provides an expression cassette comprising:
(i) a nucleotide sequence that encodes at least one AFP combination of the present invention as described herein, or
(ii) a nucleic acid sequence complementary to (i), or
(iii) both of (i) and (ii).

In further aspects, the present invention provides an expression vector comprising at least one expression cassette of the present invention as described herein.

In further aspects, the present invention provides a cell comprising at least one nucleic acid molecule, or combination of nucleic acid molecules, of the present invention as described herein. In particular embodiments, the cell comprises at least one expression cassette or at least one expression vector of the present invention as described herein.

In a further aspect, the present invention relates to a method for preparing a polypeptide of interest (POI) comprising in its amino acid sequence one or more non-canonical amino acid (ncAA) residues. Said method comprises expressing the POI in a cell of the present invention in the presence of said one or more ncAAs, wherein the cell comprises:
(i) at least one AFP comprising a RNA-TP segment and at least one AFP comprising an O-RS segment as described herein;
(ii) a POI-encoding nucleotide sequence (CS^{POI}) wherein said one or more ncAA residues of the POI are encoded by selector codon(s),
(iii) a targeting nucleotide sequence (TN) that is functionally linked to the CS^{POI} and is able to interact with an RNA-TP segment of at least one of the AFPs in the cell;
(iv) one or more orthogonal tRNA^{ncAA} (O-tRNA^{ncAA}) molecules which carry the anticodon(s) complementary to the selector codon(s) of the CS^{POI}, and wherein said O-tRNA^{ncAA} molecules together with one or more O-RS segments of the AFPs in the cell form one or more orthogonal O-RS/O-tRNA^{ncAA} pairs which allow for the introduction of said one or more ncAA residues into the amino acid sequence of the POI;
and wherein the method optionally further comprises recovering the expressed POI.

Said at least one AFP comprising a RNA-TP segment and said at least one AFP comprising an O-RS segment recited in (i) can be one and the same type of AFP, i.e. an AFP comprising both a RNA-TP segment and an O-RS segment. Alternatively, said at least one AFP comprising a RNA-TP segment and said at least one AFP comprising an O-RS segment recited in (i) can be different AFPs.

In a further aspect, the present invention relates to a method for preparing a polypeptide of interest (POI) comprising in its amino acid sequence one or more non-canonical amino acid (ncAA) residues. Said method comprises expressing the POI in a cell of the present invention in the presence of said one or more ncAAs, wherein the cell comprises:
(i) RNA-TP/O-RS fusion proteins of the present invention as described herein;
(ii) a POI-encoding nucleotide sequence (CS^{POI}) wherein said one or more ncAA residues of the POI are encoded by selector codon(s),
(iii) a targeting nucleotide sequence (TN) that is functionally linked to the CS^{POI} and is able to interact with an RNA-TP segment of at least one of the RNA-TP/O-RS fusion proteins in the cell;
(iv) one or more orthogonal tRNA^{ncAA} (O-tRNA^{ncAA}) molecules which carry the anticodon(s) complementary to the selector codon(s) of the CS^{POI}, and wherein said O-tRNA^{ncAA} molecules together with one or more O-RS segments of the RNA-TP/O-RS fusion proteins in the cell form one or more orthogonal O-RS/O-tRNA^{ncAA} pairs which allow for the introduction of said one or more ncAA residues into the amino acid sequence of the POI;
and wherein the method optionally further comprises recovering the expressed POI.

In a further aspect, the present invention relates to a method for preparing a polypeptide of interest (POI) comprising in its amino acid sequence one or more non-canonical amino acid (ncAA) residues. Said method comprises the steps of:
(a) expressing in a cell one or more AFPs comprising at least one RNA-TP segment and one or more AFPs comprising at least one O-RS segment as described herein;
(b) expressing in said cell one or more orthogonal tRNA^{ncAA} (O-tRNA^{ncAA}) molecules, wherein
   said orthogonal tRNA^{ncAA} molecules and one or more of the O-RS segments of the AFPs in the cell form one or more orthogonal aminoacyl tRNA synthetase/tRNA^{ncAA} (O-RS/O-tRNA^{ncAA}) pairs,
   said O-RS/O-tRNA^{ncAA} pairs allow for introducing said one or more ncAA residues into the amino acid sequence of said POI,
wherein steps (a) and (b) can be concomitantly or sequentially in any order;
(c) then, expressing said POI in said cell in the presence of said one or more ncAAs, wherein
   the POI-encoding nucleotide sequence (CS^{POI}) comprises one or more selector codons encoding said one or more ncAA residues,
   said selector codons match the anticodons of said one or more O-tRNA^{ncAA} molecules;
   said CS^{POI} is functionally linked to a targeting nucleotide sequence (TN), thus forming a CS^{POI}/TN fusion sequence,
   said CS^{POI}/TN fusion sequence is able to interact, via its TN, with an RNA-TP segment of at least one of the AFPs in the cell;
and
(d) optionally recovering the expressed POI.

In a further aspect, the present invention relates to a method for preparing a polypeptide of interest (POI) comprising in its amino acid sequence one or more non-canonical amino acid (ncAA) residues. Said method comprises the steps of:
(a) expressing in a cell RNA-TP/O-RS fusion proteins of the present invention as described herein;
(b) expressing in said cell one or more orthogonal tRNA^{ncAA} (O-tRNA^{ncAA}) molecules, wherein
   said orthogonal tRNA^{ncAA} molecules and one or more of the O-RS segments of the RNA-TP/O-RS fusion proteins in the cell form one or more orthogonal aminoacyl tRNA synthetase/tRNA^{ncAA} (O-RS/O-tRNA^{ncAA}) pairs,
   said O-RS/O-tRNA^{ncAA} pairs allow for introducing said one or more ncAA residues into the amino acid sequence of said POI,
wherein steps (a) and (b) can be concomitantly or sequentially in any order;
(c) then, expressing said POI in said cell in the presence of said one or more ncAAs, wherein
   the POI-encoding nucleotide sequence (CS^{POI}) comprises one or more selector codons encoding said one or more ncAA residues,
   said selector codons match the anticodons of said one or more O-tRNA^{ncAA} molecules;
   said CS^{POI} is functionally linked to a targeting nucleotide sequence (TN), thus forming a CS^{POI}/TN fusion sequence,
   said CS^{POI}/TN fusion sequence is able to interact, via its TN, with an RNA-TP segment of at least one of the RNA-TP/O-RS fusion proteins in the cell;
and
(d) optionally recovering the expressed POI.

In a further aspect, the present invention relates to a nucleic acid molecule comprising:
(i) a nucleotide sequence (CS^{POI}) that encodes a polypeptide of interest (POI), said POI comprising one or more, identical or different, non-canonical amino acid (ncAA) residues which are encoded in the CS^{POI} by selector codons, and
(ii) a targeting nucleotide sequence (TN), wherein an RNA molecule comprising said TN is able to interact via said TN with an RNA-targeting polypeptide (RNA-TP).

In a further aspect, the present invention relates to a kit for preparing a polypeptide of interest (POI) having at least one non-canonical amino acid (ncAA) residue, the kit comprising:
at least one ncAA, or salt thereof, corresponding to the at least one ncAA residue of the POI, and
at least one expression vector of the present invention as described herein.

Said expression vector comprises at least one expression cassette comprising:
(i) a nucleotide sequence that encodes at least one RNA-TP/O-RS fusion protein of the present invention, at least one AFP of the present invention, or at least one AFP combination of the present invention, or
(ii) a nucleic acid sequence complementary to (i), or
(iii) both of (i) and (ii).

### BRIEF DESCRIPTION OF THE FIGURES

FIG. 1 shows a schematic representation of the spatial separation of the components which allow for orthogonal translation so as to decode a specific stop codon in a uniquely tagged mRNA. (A) Conventional expression of the synthetase PylRS leads to aminoacylation of its cognate stop codon suppressor tRNA^{Pyl} with a custom designed ncAA. This leads to site-specific ncAA incorporation whenever the respective stop codon occurs in mRNA of the POI. Given that many endogenous mRNAs terminate on the same stop codon, utilizing this approach in the cytoplasm potentially leads to misincorporation of the ncAA into unwanted proteins (left box). (B) To avoid this, the present invention allows that the mRNA encoding the POI and the orthogonal aminoacyl-tRNA synthetase (e.g., PylRS) can be brought into close proximity to one another through the use of an RNA-targeting polypeptide segment (e.g., MCP) and assemblers (APs). This allows for spatial enrichment of all components so as to create an OT assembly (“OT organelle”), including the mRNA encoding the POI, the orthogonal aminoacyl-tRNA synthetase, the tRNA, and ribosomes (right box). Here aminoacylated tRNA^{Pyl} is particularly available in direct proximity of the OT organelle, so that particularly here stop codon suppression (of the POI mRNA) can occur. This leads to a selective suppression of stop codons (and thus expression) of the POI mRNA over corresponding stop codons in mRNAs that are not targeted to the OT assembly. While in (A) GCE occurs stop codon-specific, in (B) it should occur stop codon-specific and mRNA-specific.

FIG. 2A shows a schematic representation of different assembler classes. B=bimolecular MCP::PylRS fusion, P1=fusions to FUS and EWSR1, P2=SPD5, K1=truncation of kinesin KIF13A (KIF13A_{1-411,ΔP390}), K2=truncation of kinesin KIF16B (KIF16B₁₋₄₀₀) and combinations thereof (K1::P1, K1::P2, K2::P1, K2::P2).

FIG. 2B shows a schematic representation of the dual-color reporter. mRNAs encoding the fluorescent proteins GFP and mCherry, containing stop codons at permissive sites, are expressed from one plasmid, each with its own CMV promoter, ensuring a constant ratio of mRNA throughout each experiment. The mRNA of the mCherry reporter is tagged with two MS2 RNA stem-loops (“ms2”, also referred to as MS2-tag herein), mRNA(mCherry)::ms2. In the presence of ncAA and tRNA^{Pyl}, in the case of cytoplasmic PylRS, both GFP^{39STOP} and mCherry^{185STOP} are produced, leading to a diagonal in fluorescence flow cytometry (FFC) analysis (left box). However, under the same conditions, orthogonal translation in OT organelles enables selective stop codon suppression of mRNA(mCherry)::ms2, resulting in an mCherry-positive and GFP-negative population (drawn schematically as a vertical population in the right box). In both schemes, non-transfected HEK293T cells are represented by a gray circle at the bottom.

FIG. 2C shows the selectivity and relative efficiency of various exemplary OT systems. For all experiments the indicated constructs were co-expressed with tRNA^{Pyl} (anticodon corresponding to the indicated codon) and the dual reporter (GFP^{39STOP}, mCherrys^{185STOP}::ms2). GCE was performed in presence of the indicated ncAAs, and cells were analyzed by FFC. The dark gray bars (normalized to cytoplasmic PylRS) represent the fold change in the ratios r of the mean fluorescence intensities of mCherry versus GFP (derived from FFC, see FIG. 2D, E) for all the systems tested. The light-gray bars represent the relative efficiency as defined by the mean fluorescence intensity of mCherry for each condition divided by cytoplasmic PylRS control (derived from FFC, see FIG. 2D, E). Shown are the mean values of at least three independent experiments; error bars represent the SEM. The box highlights the best performing OT organelle (OT^{K2::P1})

FIG. 2D shows the results of the FFC analysis of the dual-color reporter expressed with the four indicated systems in transfected HEK293T cells and tRNA^{Pyl} in the presence of the ncAA SCO, a lysine derivative with a cyclooctyne side chain. Highly selective and efficient orthogonal translation was observed for the OT assembly (the black arrow indicates a bright, highly mCherry-positive population). Shown in the dot plots are the sums of at least three independent experiments. Axes indicate fluorescence intensity in arbitrary units.

FIG. 2E shows FFC plots for the OT assembly selectively translating Opal and Ochre codons only of recruited mRNA(mCherry^{185TGA})::ms2 and mRNA(mCherry^{185TAA})::ms2, respectively.

FIG. 3 shows a schematic representation of the constructs composing the following systems: PylRS, MCP::PylRS, FUS::MCP::PylRS and LcK::FUS::PylRS⋅LcK::EWS::MCP.

FIG. 4 shows the flow cytometry analysis of the dual reporter expression with the 4 different systems depicted in FIG. 3. HEK293T cells were transfected with constructs encoding the dual reporter, tRNA, LcK::FUS::PylRS and LcK::EWS::MCP or PylRS, MCP::PylRS, FUS::MCP::PylRS and pcDNA3.1. Shown is the sum of at least three independent experiments. Axes indicate fluorescence intensity in arbitrary units.

FIG. 5 shows a bar plot with the ratios of the mean fluorescence intensity of mCherry vs. GFP fluorescence for all the tested systems. Plots represent mean values of at least 3 biological replicates, error bars indicate standard error of means.

FIG. 6 provides an overview of different approaches of the present invention for generating OT organelles, which target to the surface of different intra-cellular structures. Different constructs are expressed and the results of the respective fluorescence flow cytometry (FFC) analyses are shown. On top of the figure the dual color reporter construct GFP^{39TAG}.mCherry^{185TAG}::ms2 (see also FIG. 2B) as applied in each of the schematically illustrated experiments A to G is depicted and a schematic illustration of different targeted cellular compartments is shown. Control experiments performed without the effector polypeptide MCP (-MCP) are also illustrated for each of the experiments A to G:

A: OT organelle targeted to microtubules and obtained by expressing the system KIF16B₁₋₄₀₀::FUS::PylRS⋅KIF16B₁₋₄₀₀::EWSR1::MCP or the construct KIF16B₁₋₄₀₀::FUS::PylRS (control);

B: OT organelle targeted to microtubule plus ends and obtained by expressing the constructs EB1::FUS::MCP::PylRS or EB1::FUS::PylRS (control).

C: OT organelle targeted to plasma membrane and obtained by expressing the system LcK::FUS::PylRS⋅LcK::EWSR1::MCP or the construct LcK::FUS::PylRS (control).

D: OT organelle targeted to mitochondrial membrane and obtained by expressing the system TOM20₁₋₇₀::FUS::PylRS⋅TOM20₁₋₇₀::EWSR1::MCP or the construct TOM20₁₋₇₀::FUS::PylRS (control).

E: OT organelle targeted to nuclear membrane and obtained by expressing the system CG1::FUS::PylRS⋅CG1::EWSR1::MCP or the construct CG1::FUS::PylRS (control).

F (left side): OT organelle targeted to Golgi membrane and obtained by expressing the system EBAG9₁₋₂₉::FUS::PylRS⋅EBAG9₁₋₂₉::EWSR1::MCP or the construct EBAG9₁₋₂₉::FUS::PylRS (control).

F (right side): OT organelle targeted to Golgi membrane and obtained by expressing the system CMP Sia Tr::FUS::PylRS⋅CMP Sia Tr::MCP or the construct CMP Sia Tr::FUS::PylRS (control).

G: OT organelle targeted to ER membrane and obtained by expressing the system P450 2C1₁₋₂₇::FUS::PylRS⋅P450 2C1₁₋₂₇::EWSR1::MCP or the construct P450 2C1₁₋₂₇::FUS::PylRS (control).

FIG. 7 provides an overview of different approaches of the present invention for recruiting RNA using the interaction of different RNA loops and respective RNA targeting proteins. The results of the respective fluorescence flow cytometry (FFC) analyses are shown and compared to the respective analysis as obtained for non-targeted PylRS alone:

A: System ms-2-MCP incorporates the ms2 loops in the UTR of an mRNA molecule and recruits the mRNA with the MCP protein into the artificial organelle.

B: System boxB-λ_{N22} incorporates the boxB loops in the UTR of an mRNA molecule and recruits the mRNA with the λ_{N22} protein into the artificial organelle C: System pp7-PCP incorporates the pp7 loops in the UTR of an mRNA molecule and recruits the mRNA with the PCP protein into the artificial organelle.

FIG. 8 illustrates a further approach of the present invention for generating OT organelles which will work on the surface of different cellular structures. Here the targeting to plasma membrane is exemplified. The particular approach is characterized by the pairwise incorporation of so-called synthetic heterodimeric-coiled coil peptides SYNZIP1 and SYNZIP2 fused into the system LcK::FUS::SYNZIP1::PylRS⋅EWSR1::SYNZIP2::MCP; upon expression SYNZIP1 and 2 pair and recruit MCP to a plasma membrane based OT organelle which in turn enables the selective orthogonal translation of a subsequently recruited mRNA comprising the ms2 targeting nucleotide loops. Selective translation is illustrated by the results of the respective FFC analysis (A). In a comparative approach with the system LcK::FUS::PylRS⋅EWSR1::SYNZIP2::MCP, wherein SYNZIP1 is missing, no selectivity of translation could be observed (B).

### DETAILED DESCRIPTION OF THE INVENTION

Unless otherwise defined herein, scientific and technical terms as used in the context of the present invention shall have the meanings that are commonly understood by those of ordinary skill in the art. The meaning and scope of the terms should be clear. However, in the event of any latent ambiguity, definitions provided herein take precedent over any dictionary or extrinsic definition. Further, unless otherwise required by context, singular terms shall include pluralities and plural terms shall include the singular.

If not otherwise stated, nucleotide sequences are depicted herein in the 5′ to 3′ direction. If not otherwise stated, amino acid sequences are depicted herein in the direction from N-terminus to C-terminus.

If not otherwise stated, the polypeptide of interest (POI) that is translationally expressed by the OT system according to the present invention comprises one or more ncAA residues which are encoded in the nucleotide sequence encoding the POI (CS^{POI}) by selector codons.

1. Fusion Proteins

1.1. General

The fusion proteins of the invention may be construed in different manner.

A first type includes fusion proteins wherein at least two types of effector polypeptides (EPs), comprising at least one RNA-TP and at least one O-RS, are comprised by one and the same fusion protein (also designated as RNA-TP/O-RS fusion proteins).

A second type includes fusion proteins which comprise at least one assembler polypeptide (AP) and at least one type of EP selected from RNA-TP segments and O-RS segments (also designated AFPs). In particular, AFPs can comprise both RNA-TP and O-RS segments, such as one or more RNA-TP segments and one or more O-RS segments in any sequential order, in addition to the at least one type of AP. Thus, AFPs in particular are selected from the following fusion protein types (segments functionally linked in any order within the polypeptide chain; one or more segments of the same type in any order within the polypeptide chain):

(RNA-TP/AP)

(O-RS/AP)

(RNA-TP/O-RS/AP)

APs are selected from IC-TPs and PSPs, and may be composed of one or more IC-TPs and/or one or more of PSPs in any sequential order. Thus, AFPs more particularly are selected from the following fusion protein types (segments functionally linked in any order within the polypeptide chain; one or more segments of the same type in any order within the polypeptide chain):

(RNA-TP/IC-TP)

(O-RS/IC-TP)

(RNA-TP/O-RS/IC-TP)

(RNA-TP/PSP)

(O-RS/PSP)

(RNA-TP/O-RS/PSP)

(RNA-TP/PSP/IC-TP)

(O-RS/PSP/IC-TP)

(RNA-TP/O-RS/PSP/IC-TP)

APs and/or EPs may also comprise (as part of the fusion protein) heterooligomer forming, in particular heterodimer forming polypeptide segments, like in particular synthetic coiled coil SYNZIP peptides. AFP combinations comprising such interacting SYNZIP pairs distributed between members of said AFP combination, so that each AFP comprises merely one member of such interacting SYNZIP pair are particular embodiments.

The term “segment” as used herein in the context of fusion proteins indicates that the thus designated element (e.g., RNA-TP, O-RS, IC-TP, PSP, SYNZIP) is part of the fusion protein, i.e. linked to the remainder of the fusion protein. The segments of the fusion proteins of the invention are functionally linked, i.e. linked such that they still function as RNA-TP, O-RS, IC-TP and PSP or SYNZIP, respectively. Said linkage is preferably covalent, and in particular is a peptidic linkage.

For example, the RNA-TP segment comprised in the fusion proteins of the present invention is a segment of the fusion protein that is derived from, and functions in the context of the fusion protein as, an RNA-TP, thus allowing the fusion protein to interact with (bind to) the targeted RNA, wherein said interaction is expediently a specific one. Thus, an RNA-TP segment may comprise the (entire) amino acid sequence, or a functional fragment, of an RNA-targeting polypeptide as described herein.

Analogously, an O-RS segment comprised by the fusion proteins of the present invention is a segment of the fusion protein that is derived from, and functions in the context of the fusion protein as, an O-RS, thus conferring to the fusion protein O-RS enzymatic activity, that is the ability to catalyze the aminoacylation of an O-tRNA with an ncAA. Thus, an O-RS segment may comprise the (entire) amino acid sequence, or a functional fragment, of an O-RS as described herein.

The assembler fusion proteins (AFPs) described herein comprise at least one polypeptide segment acting as an assembler (AP). As used herein the term AP refers to any polypeptide segment that allows for enrichment of AFPs comprising said segment at spatially distinct sites within a living cell. Expediently said spatially distinct sites are located within, or directly adjacent to, the cytoplasm of the cell and readily accessible to the translational machinery of the cell (which includes canonical aminoacylated tRNAs, translation factors, ribosomal subunits, etc.) as well as the O-tRNAs which allow for the introduction of the ncAA residues into the POI.

There are different types of polypeptide segments which can serve as APs in the present invention. One type of APs are polypeptide segments which are derived from, and function in the context of the fusion protein as, intracellular targeting polypeptides (IC-TPs). These IC-TP segments may comprise the (entire) amino acid sequence, or a function fragment, of an IC-TP. IC-TPs target, and thus become locally enriched at, intracellular structural elements within, or directly adjacent to, the cytoplasm. Examples of such structural elements include microtubules, the cytoplasmic side of membranes such as the cell membrane, the nuclear membrane, the mitochondrial membrane, the Golgi membrane, the ER membrane, etc.

Accordingly, in particular embodiments, the fusion protein of the present invention comprises at least one IC-TP segment that targets, and facilitates local enrichment of the fusion protein at, microtubules, in particular the plus end or the minus end of the microtubules). For instance, dyneins and kinesins (proteins of the dynein or kinesin family of proteins), and functional fragments and mutants thereof, can be used as IC-TPs for such function.

In further particular embodiments, the fusion protein of the present invention comprises at least one IC-TP segment that is derived from, and functions as, a membrane anchor. For example, the fusion protein of the present invention comprises at least one IC-TP segment that targets, and facilitates local enrichment of the fusion protein at, the (inner) cell membrane (in particular the cytoplasmic side of the cell membrane). In another example, the fusion protein of the present invention comprises at least one IC-TP segment that targets, and facilitates local enrichment of the fusion protein at, the (outer) nuclear membrane (in particular the cytoplasmic side of the nuclear membrane). In further particular embodiments, the fusion protein of the present invention comprises at least one IC-TP segment that targets, and facilitates local enrichment of the fusion protein at, the outer mitochondrial membrane (in particular the cytoplasmic side of the mitochondrial membrane). In further particular embodiments, the fusion protein of the present invention comprises at least one IC-TP segment that targets, and facilitates local enrichment of the fusion protein at, the outer ER membrane (in particular the cytoplasmic side of the ER membrane). In further particular embodiments, the fusion protein of the present invention comprises at least one IC-TP segment that targets, and facilitates local enrichment of the fusion protein at, the outer Golgi membrane (in particular the cytoplasmic side of the Golgi membrane). For instance, the transmembrane domain of membrane proteins, and functional fragments and mutants thereof, can be used as IC-TPs for such function.

Polypeptides which target, and thus become locally enriched at, intracellular structural elements as described above, are known in the art and are useful as IC-TPs in the present invention. Specific examples of suitable IC-TPs include, but are not limited to:
optionally truncated kinesin polypeptides which constitutively move towards, and become locally enriched at, microtubule-plus ends in living cells, for example optionally truncated kinesin family member 16B (KIF16B), e.g. optionally truncated Homo sapiens KIF16B (Uniprot: Q96L93), in particular the fragment covering KIF16B amino acid residues 1-400 (KIF16B₁₋₄₀₀) comprising the amino acid sequence of SEQ ID NO:20; or optionally truncated kinesin family member 13A (KIF13A), e.g. optionally truncated Homo sapiens KIF13A (Uniprot: Q9H1H9), in particular the KIF13A fragment covering amino acid residues 1-411 wherein P390 is deleted (KIF13A_{1-411,Δ390}) comprising the amino acid sequence of SEQ ID NO:22; polypeptides EB1, a microtubule tip binding protein, that binds to growing microtubule plus ends (Nehlig A, Molina A, Rodrigues-Ferreira S, Honoré S, Nahmias C. Regulation of end-binding protein EB1 in the control of microtubule dynamics. Cell Mol Life Sci. 2017; 74(13):2381-2393. doi:10.1007/s00018-017-2476-2) (Uniprot: Q15691) and hence targets the organelle to microtubule-plus ends and comprising the amino acid sequence of SEQ ID NO:302
polypeptides targeting the outer mitochondrial membrane derived from transmembrane-proteins such as, e.g., optionally truncated translocase of outer mitochondrial membrane 20 (TOMM20), for example optionally truncated Homo sapiens TOMM20 (Uniprot: Q15388), in particular the fragment covering amino acid residues 1-70 of TOMM20 (TOMM20₁₋₇₀) comprising the amino acid sequence of SEQ ID NO:24;
cell membrane-targeting polypeptides derived from transmembrane-proteins such as, e.g., lymphocyte-specific protein tyrosine kinase (LcK; e.g., Mus musculus LcK, Uniprot: P06240), CD4 (e.g., Mus musculus CD4, Uniprot: P06332), FRB (similar to Homo sapiens mTOR; Uniprot: P42345), CD28 (e.g., Mus musculus CD28, Uniprot: P31041) and combinations thereof, in particular polypeptides comprising the amino acid sequence of SEQ ID NO:26, SEQ ID NO:28 or SEQ ID NO:30;
polypeptides CG1, a nucleoporin that binds to the cytoplasmic side of the nuclear pore complex (Fernandez-Martinez J, Kim S J, Shi Y, et al. Structure and Function of the Nuclear Pore Complex Cytoplasmic mRNA Export Platform. Cell. 2016; 167(5):1215-1228.e25. doi:10.1016/j.cell.2016.10.028) (also designated Nup42) (Uniprot: 015504) targeting the cytoplasmic side of the nuclear membrane comprising the amino acid sequence of SEQ ID NO:304
polypeptides EBAG9, Golgi membrane protein with one transmembrane helix (Engelsberg A, Hermosilla R, Karsten U, Schulein R, Dörken B, Rehm A. The Golgi protein RCAS1 controls cell surface expression of tumor-associated O-linked glycan antigens. J Biol Chem. 2003; 278(25):22998-23007. doi:10.1074/jbc.M301361200 (Uniprot: 000559) targeting the cytoplasmic side of the Golgi membrane comprising the amino acid sequence of SEQ ID NO:292 (full length) or comprising the first 29 N-terminal amino acid residues of SEQ ID NO:294; or polypeptides CMP Sia Tr, the CMP sialic acid transporter, a Golgi protein with 10 transmembrane helices (Eckhardt M, Gotza B, Gerardy-Schahn R. Membrane topology of the mammalian CMP-sialic acid transporter. J Biol Chem. 1999; 274(13):8779-8787. doi:10.1074/jbc.274.13.8779) (Uniprot: P78382) targeting the cytoplasmic side of the Golgi membrane comprising the amino acid sequence of SEQ ID NO:296
polypeptide fragments of P450 2C1, a endoplasmic reticulum resident protein (Fazal F M, Han S, Parker K R, et al. Atlas of Subcellular RNA Localization Revealed by APEX-Seq. Cell. 2019; 178(2):473-490.e26. doi:10.1016/j.cell.2019.05.027) (Uniprot: P78382) targeting the cytoplasmic side of the ER membrane in particular a fragment comprising the N-terminal first 27 (SEQ ID NO:298); or the first 29 (SEQ ID NO:300); amino acid residues
The transmembrane protein stomatin-like protein 3 (SLP-3) (membrane comprising the amino acid sequence of SEQ ID NO:310; aa 1-59 (Homo sapiens, Uniprot: Q8TAV4), localizing to the plasma membrane and vesicular membranes (Lapatsina L, Jira J A, Smith E S, et al. Regulation of ASIC channels by a stomatin/STOML3 complex located in a mobile vesicle pool in sensory neurons. Open Biol. 2012; 2(6):120096. doi:10.1098/rsob.120096)

as well as functional fragments and mutants of these polypeptides. Said functional fragments and mutants may have at least 60%, at least 70%, at least 80%, at least 90%, at least 95%, at least 96%, at least 97%, at least 98% or at least 99% amino acid sequence identity to the amino acid of the polypeptide they are derived from.

A further type of APs are polypeptide segments, which are derived from, and function in the context of the fusion protein as, phase separation polypeptides (PSPs). PSPs are polypeptides, which have the ability to self-assemble in the cytoplasm of a cell so as to create sites of high local concentration in the cytoplasm. Specifically, PSPs are able to drive phase separation (in particular liquid-liquid phase separation) leading to the formation of membrane-less compartments in the cytoplasm. Said compartments may take the form of droplets, aggregates, condensates or a dense phase. In particular, PSPs include intrinsically disordered proteins (IDPs) which are an important class of proteins that drive phase separation (see, e.g., Alberti et al., Bioessays 2016, 38:959-968 and references cited therein such as Patel et al., Cell 2015, 162:1066-1077; Han et al., Cell 2012, 149:768-779; Kato et al., Cell 2012, 149:753-767). There are three different classes of ICPs, proteins of each, or functional fragments or mutants thereof, can be used as PSPs in the present invention. One prominent class of IDPs contains so called prion-like domains which are devoid of charges and contain polar amino acid residues (Q, N, S, G) with interspersed aromatic residues (F, Y). See, e.g., Malinovska et al., Biochim Biophys Acta 2013, 1834:918-931; Alberti et al., 2009, Cell 137:146-158, Malinovska et al., Prion 2015, 9:339-346. Another class of IDPs is also characterized by low sequence complexity but frequently contains acidic and basic amino acid side chains, e.g. RGG repeat containing IDPs such as Ddx4. See Nott et al., Cell 2015, 57:936-947. Specific examples of suitable IC-TPs include, but are not limited to:
spindle-defective protein 5 (SPD5) (e.g., Caenorhabditis elegans SPD5; Uniprot: P91349), in particular a polypeptide comprising the amino acid sequence of SEQ ID NO:32;
fused-in sarcoma (FUS) (e.g., Homo sapiens FUS; Uniprot: P35637), in particular a polypeptide comprising the amino acid sequence of SEQ ID NO:34;
Ewing sarcoma breakpoint region 1 (EWSR1) (e.g., Homo sapiens EWSR1; Uniprot: Q01844), in particular a polypeptide comprising the amino acid sequence of SEQ ID NO:36;
ATP-dependent RNA helicase laf-1 (RGG domain, 1-168, LAF-1 membrane comprising the amino acid sequence of SEQ ID NO:308); (Caenorhabditis elegans, Uniprot: DOPV95), (Schuster B S, Reed E H, Parthasarathy R, et al. Controllable protein phase separation and modular recruitment to form responsive membraneless organelles. Nat Commun. 2018; 9(1):2985. Published 2018 Jul. 30. doi:10.1038/s41467-018-05403-1)

as well as functional fragments and mutants of these polypeptides. Said functional fragments and mutants may comprise at least 60%, at least 70%, at least 80%, at least 90%, at least 95%, at least 96%, at least 97%, at least 98% or at least 99% amino acid sequence identity to the amino acid of the polypeptide they are derived from.

The number of APs comprised by fusion proteins of the present invention is not particularly limited, i.e. a fusion protein may comprise 1, 2, 3, 4, 5, 6, 7, 8, 9, 10 or more same or different APs. Fusion proteins of the present invention which comprise at least one AP selected from IC-TP segments and at least one AP selected from PSP segments are particularly preferred. Likewise, the number of RNA-TP segments is not particularly limited and may be independently selected from 1, 2, 3, 4, 5 or more, as for example 6, 7, 8, 9 or 10, different or same RNA-TP segments. Likewise, the number of O-RS segments is not particularly limited and may be independently selected from 1, 2, 3, 4, 5 or more, as for example 6, 7, 8, 9 or 10, different or same O-RS segments. This applies to both AFPs as well as to RNA-TP/O-RS fusion proteins. The number of segments in the fusion proteins of the present invention of course influences the size of the fusion protein that is not particularly limited but typically less than 3500 amino acid residues, such as less than 3000 amino acid residues.

The order of the segments within the fusion proteins of the invention is not particularly limited either. The RNA-TP, O-RS and/or AP segments may thus be functionally linked in any order.

Examples of RNA-TP/O-RS fusion protein structures (comprising both types of EP segments) include, but are not limited to,
[RNA-TP]ₓ-[O-RS]_{y}
[O-RS]_{y}-[RNA-TP]ₓ

wherein x and y, independently of each other, are integers selected from 1, 2, 3, 4 and 5; “-” designates a peptidic linkage.

[RNA-TP]ₓ for x>2 may include the same or different RNA-TP segments. [O-RS]_{y} for y≥2 may include the same or different O-RS segments.

Examples of RNA-TP/O-RS fusion protein structures include, but are not limited to:

[IC-TP]ₘ-[EP]ₒ

[EP]ₒ-[IC-TP]ₘ

[PSP]ₙ-[EP]ₒ

[EP]ₒ-[PSP]ₙ

[IC-TP]ₘ-[EP]ₒ-[PSP]ₙ

[PSP]ₙ-[EP]ₒ-[IC-TP]ₘ

[IC-TP]ₘ-[PSP]ₙ-[EP]ₒ

[EP]ₒ-[PSP]ₙ-[IC-TP]ₘ

[PSP]ₙ-[IC-TP]ₘ-[EP]ₒ

[EP]ₒ-[IC-TP]ₘ-[PSP]ₙ

wherein m, n and o, independently of each other, are integers selected from 1, 2, 3, 4 or 5, or are selected from 1, 2, 3, 4, 5, 6 and “-” designates a peptidic linkage.

In a preferred embodiment “m” is the integer 1.

In another preferred embodiment “n” is an integer selected from 1 and 2.

In still another preferred embodiment “o” is an integer selected from 1, 2, 3, 4, 5 or 6 if EP is selected from RNA-TPs.

In still another preferred embodiment “o” is an integer selected from 1 or 2, if EP is selected from O-RSs.

In still another preferred embodiment of RNA-TP/O-RS fusion protein structures those are preferred wherein at least one ICT-TP takes a C- or N-terminal position within the polypeptide chain.

In still another preferred embodiment of RNA-TP/O-RS fusion protein structures those are preferred wherein at least one EP takes a C- or N-terminal position within the polypeptide chain.

In still another preferred embodiment of RNA-TP/O-RS fusion protein structures those are preferred wherein at least one ICT-TP takes a C- or N-terminal position within the polypeptide chain while at least one EP takes a N- or C-terminal position, respectively, within the polypeptide chain. Any PSP, if present in such structure, is positioned within the polypeptide chain.

[IC-TP]ₘ for m≥2 may include the same or different IC-TP segments. Preferably IC-TPs of the same functionality (targeting the same type of cellular structure (as for example same membrane type or type or organelle) are applied. [PSP]ₙ for n≥2 may include the same or different PSP segments. [EP]ₒ for o≥2 may include the same or different EPs. Where [EP]ₒ includes different EPs, for example at least one EP may be a RNA-TP segment and at least one may be an O-RS segment.

The fusion proteins of the present invention provide an orthogonal translation (OT) system wherein the one or more O-RS (segments) required for the introduction of the one or more ncAA residues into the POI are brought into spatial proximity to at least one RNA-targeting polypeptide (RNA-TP) segment. The mRNA of the POI comprises at least one targeting nucleotide sequence (TN) that is able to interact with an RNA-TP segment of at least one of the fusion proteins of the OT system. Said interaction is expediently a specific one. The RNA-TP segments of the fusion proteins of the invention are preferably mRNA-targeting polypeptide segments. The RNA-TP segment of the fusion protein and the TN of the POI mRNA are expediently chosen so as to specifically interact with (bind to) one another. Suitable pairs of RNA-TP segment and TN for this purpose can be selected from coat proteins of RNA viruses and the nucleic acid motifs bound by said coat proteins. Such viral coat proteins and protein-bound RNA motifs are known in the art.

Specific examples of suitable RNA-TPs include, but are not limited to:
MCP (coat protein of Enterobacteria phage MS2), in particular a polypeptide comprising the amino acid sequence of SEQ ID NO:14;
λ_{N22} (22 amino acid RNA-binding domain of lambda phage antiterminator protein N), in particular a polypeptide comprising the amino acid sequence of SEQ ID NO:16;
PCP (coat protein of Bacteriophage PP7, Wu B, Chao J A, Singer R H. Fluorescence fluctuation spectroscopy enables quantitative imaging of single mRNAs in living cells. Biophys J. 2012; 102(12):2936-2944. doi:10.1016/j.bpj.2012.05.017), in particular a polypeptide comprising the amino acid sequence of SEQ ID NO:306;
as well as functional fragments and mutants of these polypeptides. Said functional fragments and mutants may comprise at least 60%, at least 70%, at least 80%, at least 90%, at least 95%, at least 96%, at least 97%, at least 98% or at least 99% amino acid sequence identity to the amino acid of the polypeptide they are derived from.

Specific examples of suitable TNs include, but are not limited to:
Enterobacteria phage MS2 RNA stem-loop, in particular a polynucleotide having an RNA sequence corresponding to (encoded by) the nucleotide (DNA) sequence of SEQ ID NO:17;
BoxB (lambda phase RNA stem-loop, specific binding site of λ_{N22}), in particular a polynucleotide having an RNA sequence corresponding to (encoded by) the nucleotide (DNA) sequence of SEQ ID NO:18;
Bacteriophage pp7 RNA stem loops (Wu B, Chao J A, Singer R H. Fluorescence fluctuation spectroscopy enables quantitative imaging of single mRNAs in living cells. Biophys J. 2012; 102(12):2936-2944. doi:10.1016/j.bpj.2012.05.017) in particular a polynucleotide having an RNA sequence corresponding to (encoded by) the nucleotide (DNA) sequence of SEQ ID NO:289 or SEQ ID NO:290

as well as functional fragments and mutants thereof. Said functional fragments and mutants may comprise at least 60%, at least 70%, at least 80%, at least 90%, at least 95%, at least 96%, at least 97%, at least 98% or at least 99% nucleotide sequence identity to the polynucleotide sequences they are derived from.

Such TNs may be used as a single copy segment or as multiple copy segment composed of more than one, as for example two, three, four, five, six or more repetitive units of the TN.

MCP specifically interacts with MS2 RNA stem-loops. Thus, where the RNA-TP segment(s) of the fusion protein(s) comprise (consist of) segments which are derived from, and function as, MCP, the mRNA of the POI expediently comprises one or more MS2 RNA stem-loops, e.g. two, three, four, five or six MS2 RNA stem-loops. λ_{N22} specifically interacts with BoxB. Thus, where the RNA-TP segment(s) of the fusion protein(s) comprise (or consist of) segments which are derived from, and function as, λ_{N22}, the mRNA of the POI expediently comprises one or more BoxB motifs, e.g. one, two, three, four, five or six or more BoxB motifs. PCP specifically interacts with pp7 RNA stem-loops. Thus, where the RNA-TP segment(s) of the fusion protein(s) comprise (consist of) segments which are derived from, and function as, PCP, the mRNA of the POI expediently comprises one or more pp7 RNA stem-loops, e.g. two, three, four, five or six or more pp7 RNA stem-loops.

Several RSs have been used for genetic code expansion including the Methanococcus jannaschii tyrosyl-tRNA synthetase, E. coli tyrosyl-tRNA synthetase, E. coli leucyl-tRNA synthetase pyrrolysyl-tRNA synthetases from certain Methanosarcina (such as M. mazei, M. barkeri, M. acetivorans, M. thermophila), Methanococcoides (M. burtonii) or Desulfitobacterium (D. hafniense). Corresponding orthogonal RS/tRNA pairs have been used to genetically encode a variety of functionalities in polypeptides (Chin, Annu Rev Biochem 2014, 83:379-408; Chin et al., J Am Chem Soc 2001, 124:9026; Chin et al., Science 2003, 301:964; Nguyen et al., J Am Chem Soc 2009, 131:8720; Yanagisawa et al., Chem Biol 2008, 15:1187). Depending on the cell used for the translation of the POI, these RS can be used as O-RS in the present invention.

Pyrrolysyl tRNA synthetases (PylRSs) which can be used in methods and fusion proteins of the invention may be wildtype or genetically engineered PylRSs. Examples for wildtype PylRSs include, but are not limited to PylRSs from archaebacteria and eubacteria such as Methanosarcina maize, Methanosarcina barkeri, Methanococcoides burtonii, Methanosarcina acetivorans, Methanosarcina thermophila and Desulfitobacterium hafniense. Genetically engineered PylRSs have been described, for example, by Neumann et al. (Nat Chem Biol 2008, 4:232), by Yanagisawa et al. (Chem Biol 2008, 15:1187), and in EP2192185A1. The efficiency of genetic code expansion using PylRS can be increased by modifying the amino acid sequence of the PylRS such that it is not directed to the nucleus. To this end, the nuclear localization signal (NLS) can be removed from the PylRS or can be overridden by introducing a suitable nuclear export signal (NES). PylRSs which are used in the fusion proteins and methods of the present invention may be PylRSs lacking the NLS and/or comprising a NES as described, e.g., in WO 2018/069481.

Accordingly, examples of O-RS segment(s) which can be used in the fusion proteins of the present invention include, but are not limited to:
Methanococcus jannaschii tyrosyl-tRNA synthetase;
Escherichia coli tyrosyl-tRNA synthetase;
Escherichia coli leucyl-tRNA synthetase;
Methanosarcina mazei pyrrolysyl-tRNA synthetase;
Methanosarcina barkeri pyrrolysyl-tRNA synthetase;
Methanosarcina acetivorans pyrrolysyl-tRNA synthetase;
Methanosarcina thermophila pyrrolysyl-tRNA synthetase;
Methanococcoides burtonii pyrrolysyl-tRNA synthetase;
Desulfitobacterium hafniense pyrrolysyl-tRNA synthetase;
as well as functional (i.e., enzymatically active) fragments and mutants of these polypeptides. Said functional fragments and mutants may comprise at least 60%, at least 70%, at least 80%, at least 90%, at least 95%, at least 96%, at least 97%, at least 98% or at least 99% amino acid sequence identity to the aminoacyl tRNA synthetase they are derived from.

Particular examples of O-RS segments useful as in the present invention which are derived from M. mazei pyrrolysyl-tRNA synthetases include, but are not limited to:
O-RS segments derived from PylRS^{AF} (Methanosarcina mazei pyrrolysyl tRNA synthetase double mutant: Y306A, Y384F; Uniprot: Q8PWY1), for example O-RS segments comprising the amino acid sequence of SEQ ID NO:8;
O-RS segments derived from PylRS^{AA} (Methanosarcina mazei pyrrolysyl tRNA synthetase double mutant: N346A, C348A; Uniprot: Q8PWY1), for example O-RS segments comprising the amino acid sequence of SEQ ID NO:10;
O-RS segments derived from PylRS^{AAAF} (Methanosarcina mazei pyrrolysyl tRNA synthetase quadruple mutant: Y306A, N346A, C348A, Y384F; Uniprot: Q8PWY1), for example O-RS segments comprising the amino acid sequence of SEQ ID NO:12;
O-RS segment derived from IFRS1, a Methanosarcina mazei pyrrolysyl tRNA mutant (L305M, Y306L, L309S, N346S, C348M), for example O-RS segments comprising the amino acid sequence of SEQ ID NO:224
O-RS segment derived from CbzRS, a Methanosarcina mazei pyrrolysyl tRNA mutant (Y306M, L309G, C348T), for example O-RS segments comprising the amino acid sequence of SEQ ID NO:226
O-RS segment derived from CpkRS, a Methanosarcina mazei pyrrolysyl tRNA mutant (A302S), for example O-RS segments comprising the amino acid sequence of SEQ ID NO:228
O-RS segment derived from OMeRS, a Methanosarcina mazei pyrrolysyl tRNA mutant: (A302T, Y384F, N346V, C348W, V401L), for example O-RS segments comprising the amino acid sequence of SEQ ID NO:236

as well as functional (i.e., enzymatically active) fragments and mutants of these polypeptide segments. Said functional fragments and mutants may comprise at least 60%, at least 70%, at least 80%, at least 90%, at least 95%, at least 96%, at least 97%, at least 98% or at least 99% amino acid sequence identity to the aminoacyl tRNA synthetase they are derived from.

According to particular embodiments, wild-type and mutant M. mazei PylRSs as described herein are used for aminoacylation of tRNA with ncAAs as described in WO2012/104422 or WO2015/107064. Exemplary ncAAs for this purpose include, but are not limited to, 2-amino-6-(cyclooct-2-yn-1-yloxycarbonylamino)hexanoic acid (SCO), 2-amino-6-(cyclooct-2-yn-1-yloxyethoxycarbonylamino)hexanoid acid, 2-amino-6[(4E-cyclooct-4-en-1-yl)oxycarbonylamino]hexanoic acid (TCO), 2-amino-6[(2E-cyclooct-2-en-1-yl)oxycarbonylamino]hexanoic acid (TCO*), 2-amino-6-(prop-2-ynoxycarbonylamino)hexanoic acid (PrK) and 2-amino-6-(9-biocyclo[6.1.0]non-4-ynylmethoxycarbonylamino)hexanoid acid (BCN).

In another embodiment of the present invention, the above-mentioned AP (IC-TP and PSP) segments and/or the above mentioned EP (RNA-TP and O-RS) segments, independently of each other, may be further combined with natural or, more particularly, synthetic protein segments, which induce and control macromolecular interactions. In particular, such further protein segments are operably fused into the polypeptide chain of an AFP of the invention. One or more, like 2, 3, 4, 5, 6, 7, 8, 9 or 10, preferably however one such protein segment may be operably fused into a single AFP of the invention. Fusion into the AFP polypeptide chain should be such that the activity of the other polypeptide segments, AP and EP, is substantially unaffected, in particular not inhibited (i.e. AP and EP remain operable), while the ability of the additional polypeptide segment to induce and control macromolecular interactions is retained. Described in literature are so-called SYNZIP peptides, forming multimeric structures. Of particular interest in the context of the invention are SYNZIPs having the ability to form specific heterodimeric coiled-coil protein structures. Such SYNZIPs are pairs of synthetic peptides capable of interacting with each other and are used to induce and control macromolecular interactions. Non-limiting examples are the pairs SYNZIP 1:2; SYNZIP 3:4 and SYNZIP 5:6. Particularly preferred according to the invention is the heterospecific coiled-coil pair SYNZIP2:SYNZIP1 as described by Reinke, A. W., Grant, R. A., Keating, A. E. (2010) J Am Chem Soc 132 6025-6031 (SYNZIP 1: SEQ ID NO:312; SYNZIP 2: SEQ ID NO:314, SYNZIP 3: SEQ ID NO:316; SYNZIP 4: SEQ ID NO:318, as well as functional fragments and mutants of these SYNZIP polypeptides. Said functional fragments and mutants may comprise at least 60%, at least 70%, at least 80%, at least 90%, at least 95%, at least 96%, at least 97%, at least 98% or at least 99% amino acid sequence identity to the amino acid of the polypeptide they are derived from). As a pairwise use is required to induce macromolecular interaction, these SYNZIPs are preferably used pairwise in AFP combinations as described herein. By the interaction of such SYNZIP pairs integrated in different AFP fusion proteins the formation of OT organelles according to the present invention may be further supported.

In still another embodiment of the present invention a fusion protein of the invention may be further modified by introducing into (fusing of) at least one so-called “epitope tag”, i.e. a short oligopeptide sequence, which serves as antibody binding sites, useful for detecting/quantifying the expressed fusion products of the invention. Non-limiting examples of such tags are the following:

VSV-G: Vesicular stomatitis virus glycoprotein epitope tag (SEQ ID NO:680)

HA: Human influence hemagglutinin epitope tag (SEQ ID NO:682)

Myc: Human c-Myc proto-oncogene epitope tag (SEQ ID NO:684)

1.2 Particular Examples of AFP Constructs of the Invention

Each individual exemplified construct may be construed in the N->C or C->N direction. The depicted schemes are given in the N->C direction.

In the case of segment blocks [IC-TP]ₘ, [PSP]ₙ, [O-RS]_{y} and [RNA-TP]ₓ, wherein m, n, y or x are an integer >1, the repetitive segments within such block may be identical or different, preferably identical.

The segments [IC-TP], [PSP], [O-RS], [RNA-TP]ₓ, and [SYNZIP] as applied therein may be prepared from the respective examples of segments described above in section 1.1.

1.2.1. Intracellular Structure-Targeting Monofunctional AFPs

1.2.1.1 Intracellular Structure-Targeting Monofunctional AFPs (i.e. Comprising One Type of EP) Individually preferred examples thereof are:

[IC-TP]ₘ-[O-RS]_{y} with m=1 or 2, preferably 1; y=1 or 2, preferably 1;

[IC-TP]ₘ-[RNA-TP]ₓ with m=1 or 2, preferably 1; x=1, 2, 3, 4, 5 or 6, preferably 2, 3 or 4;

[IC-TP]ₘ-[PSP]ₙ-[O-RS]_{y} with m=1 or 2, preferably 1; n=1, 2 or 3, preferably 1 or 2; y=1 or 2, preferably 1;

[IC-TP]ₘ-[PSP]ₙ-[RNA-TP]ₓ with m=1 or 2, preferably 1; n=1, 2 or 3, preferably 1 or 2; x=1, 2, 3, 4, 5 or 6, preferably 2, 3 or 4;

[IC-TP]ₘ-[O-RS₁]_{y}-[PSP]ₙ-[O-RS₂]_{y} with m=1 or 2, preferably 1; n=1, 2 or 3, preferably 1 or 2; y independently of each other=1 or 2, preferably 1; and O-RS, and O-RS₂ identical or different, preferably identical;

[IC-TP]ₘ-[PSP₁]ₙ-[O-RS]_{y}-[PSP₂]ₙ with m=1 or 2, preferably 1; n independently of each other 1, 2 or 3, preferably 1 or 2; y independently of each other=1 or 2, preferably 1; and PSP, and PSP₂ identical or different;

[IC-TP]ₘ-[RNA-TP₁]ₓ-[PSP]ₙ-[RNA-TP₂]ₓ with m=1 or 2, preferably 1; n=1, 2 or 3, preferably 1 or 2; x independently of each other=1, 2, 3, 4, 5 or 6, preferably 2, 3 or 4; and RNA-TP, and RNA-TP₂ identical or different, preferably identical;

[IC-TP]ₘ-[PSP₁]ₙ-[O-RS₁]_{y}-[PSP₂]ₙ-[O-RS₂]_{y} with m=1 or 2, preferably 1; n independently of each other 1, 2 or 3, preferably 1 or 2; y independently of each other=1 or 2, preferably 1; O-RS, and O-RS₂ identical or different, preferably identical; and PSP, and PSP₂ identical or different;

[IC-TP]ₘ-[PSP₁]ₙ-[RNA-TP₁]ₓ-[PSP₂]ₙ-[RNA-TP₂]ₓ with m=1 or 2, preferably 1; n independently of each other=1, 2 or 3, preferably 1 or 2; x independently of each other=1, 2, 3, 4, 5 or 6, preferably 2, 3 or 4; RNA-TP, and RNA-TP₂ identical or different; and PSP, and PSP₂ identical or different.

1.2.1.2 Intracellular Structure-Targeting Bifunctional AFPs (Comprising Two Types of EP)

Individually preferred examples thereof are

[IC-TP]ₘ-[O-RS]_{y}-[RNA-TP]ₓ with m=1 or 2, preferably 1; x=1, 2, 3, 4, 5 or 6, preferably 2, 3 or 4; y=1 or 2, preferably 1;

[IC-TP]ₘ-[RNA-TP]ₓ-[O-RS]_{y} with m=1 or 2, preferably 1; x=1, 2, 3, 4, 5 or 6, preferably 2, 3 or 4; y=1 or 2, preferably 1;

[IC-TP]ₘ-[PSP]ₙ-[O-RS]_{y}-[RNA-TP]ₓ with m=1 or 2, preferably 1; n=1, 2 or 3, preferably 1 or 2; x=1, 2, 3, 4, 5 or 6, preferably 2, 3 or 4; y=1 or 2, preferably 1;

[IC-TP]ₘ-[PSP]ₙ-[RNA-TP]ₓ-[O-RS]_{y} with m=1 or 2, preferably 1; n=1, 2 or 3, preferably 1 or 2; x=1, 2, 3, 4, 5 or 6, preferably 2, 3 or 4; y=1 or 2, preferably 1;

[IC-TP]ₘ-[O-RS]_{y}-[PSP]ₙ-[RNA-TP]ₓ with m=1 or 2, preferably 1; n=1, 2 or 3, preferably 1 or 2; x=1, 2, 3, 4, 5 or 6, preferably 2, 3 or 4; y=1 or 2, preferably 1;

[IC-TP]ₘ-[RNA-TP]ₓ-[PSP]ₙ-[O-RS]_{y} with m=1 or 2, preferably 1; n=1, 2 or 3, preferably 1 or 2; x=1, 2, 3, 4, 5 or 6, preferably 2, 3 or 4; y=1 or 2, preferably 1;

[IC-TP]ₘ-[PSP₁]ₙ-[O-RS]_{y}-[PSP₂]ₙ-[RNA-TP]ₓ with m=1 or 2, preferably 1; n independent of each n=1, 2 or 3, preferably 1 or 2; x=1, 2, 3, 4, 5 or 6, preferably 2, 3 or 4; y=1 or 2, preferably 1; and PSP, and PSP₂ identical or different;

[IC-TP]ₘ-[PSP₁]ₙ-[RNA-TP]ₓ-[O-RS₁]_{y}-[PSP₂]ₙ-[O-RS₂]_{y} with m=1 or 2, preferably 1; n independent of each=1, 2 or 3, preferably 1 or 2; x=1, 2, 3, 4, 5 or 6, preferably 2, 3 or 4; y independently of each other=1 or 2, preferably 1; and PSP, and PSP₂ identical or different; and O-RS, and O-RS₂ identical or different, preferably identical;

[IC-TP]ₘ-[PSP₁]ₙ-[O-RS₁]_{y}-[PSP₂]ₙ-[O-RS₂]_{y}-[RNA-TP]ₓ with m=1 or 2, preferably 1; n independent of each=1, 2 or 3, preferably 1 or 2; x=1, 2, 3, 4, 5 or 6, preferably 2, 3 or 4; y independently of each other=1 or 2, preferably 1; and PSP, and PSP₂ identical or different; and O-RS, and O-RS₂ identical or different, preferably identical.

1.2.2. No Intracellular Structure-Targeting Monofunctional AFPs

These are the same AFPs as listed above in section 1.2.1, with the only exception that the segments [IC-TP] is missing, while the segments [PSP] are retained.

1.2.3. SYNZIP Variants

These are the same AFPs as listed above in sections 1.2.1 and 1.2.2 with the only exception that at least one of the segment [IC-TP], [PSP], [O-RS₂] or [RNA-TP] is N- or C-terminally supplemented with a SYNZIP element. An AFP may contain, 1, 2, 3, 4 or 5, preferably 1 or 2, identical or different, preferably identical SYNZIPs. Non-limiting examples of such molecules are:

1.2.3.1 Monofunctional SYNZIP AFPs

Individually preferred examples thereof are:

[PSP]ₙ-[SYNZIP]-[O-RS]_{y} with y=1 or 2, preferably 1; n=1, 2 or 3, preferably 1 or 2;

[PSP]ₙ-[SYNZIP]-[RNA-TP]ₓ with; x=1, 2, 3, 4, 5 or 6, preferably 2, 3 or 4; n=1, 2 or 3, preferably 1 or 2;

[IC-TP]ₘ-[SYNZIP]-[O-RS]_{y} with m=1 or 2, preferably 1; y=1 or 2, preferably 1;

[IC-TP]ₘ-[SYNZIP]-[RNA-TP]ₓ with m=1 or 2, preferably 1; x=1, 2, 3, 4, 5 or 6, preferably 2, 3 or 4;

[IC-TP]ₘ-[PSP]ₙ-[SYNZIP]-[O-RS]_{y} with m=1 or 2, preferably 1; n=1, 2 or 3, preferably 1 or 2; y=1 or 2, preferably 1;

[IC-TP]ₘ-[PSP]ₙ-[SYNZIP]-[RNA-TP]ₓ with m=1 or 2, preferably 1 n=1, 2 or 3, preferably 1 or 2; x=1, 2, 3, 4, 5 or 6, preferably 2, 3 or 4.

1.2.3.2 Bifunctional SYNZIP AFPs

Individually preferred examples thereof are:

[IC-TP]ₘ-[O-RS]_{y}-[SYNZIP]-[RNA-TP]ₓ with m=1 or 2, preferably 1; x=1, 2, 3, 4, 5 or 6, preferably 2, 3 or 4; y=1 or 2, preferably 1;

[IC-TP]ₘ-[RNA-TP]ₓ-[SYNZIP]-[O-RS]_{y} with m=1 or 2, preferably 1; x=1, 2, 3, 4, 5 or 6, preferably 2, 3 or 4; y=1 or 2, preferably 1;

[IC-TP]ₘ-[PSP]ₙ-[SYNZIP]-[O-RS]_{y}-[RNA-TP]ₓ with m=1 or 2, preferably 1; n=1, 2 or 3, preferably 1 or 2; x=1, 2, 3, 4, 5 or 6, preferably 2, 3 or 4; y=1 or 2, preferably 1;

[IC-TP]ₘ-[PSP]ₙ-[SYNZIP]-[RNA-TP]ₓ-[O-RS]_{y} with m=1 or 2, preferably 1; n=1, 2 or 3, preferably 1 or 2; x=1, 2, 3, 4, 5 or 6, preferably 2, 3 or 4; y=1 or 2, preferably 1;

[IC-TP]ₘ-[PSP]ₙ-[SYNZIPₐ]-[O-RS]_{y}-[SYNZIP_{b}]-[RNA-TP]ₓ with m=1 or 2, preferably 1; n=1, 2 or 3, preferably 1 or 2; x=1, 2, 3, 4, 5 or 6, preferably 2, 3 or 4; y=1 or 2, preferably 1; and SYNZIPₐ and SYNZIP_{b} identical or different, preferably identical

[IC-TP]ₘ-[PSP]ₙ-[SYNZIPₐ]-[RNA-TP]r-[SYNZIP_{b}]-[O-RS]_{y} with m=1 or 2, preferably 1 n=1, 2 or 3, preferably 1 or 2; x=1, 2, 3, 4, 5 or 6, preferably 2, 3 or 4; y=1 or 2, preferably 1; and SYNZIP, and SYNZIP_{b} identical or different, preferably identical

[IC-TP]ₘ-[PSP₁]ₙ-[SYNZIP]-[RNA-TP]ₓ-[O-RS₁]_{y}-[PSP₂]ₙ-[O-RS₂]_{y} with m=1 or 2, preferably 1 n=1, 2 or 3, preferably 1 or 2; x=1, 2, 3, 4, 5 or 6, preferably 2, 3 or 4; y=1 or 2, preferably 1; and PSP, and PSP₂ identical or different; and O-RS, and O-RS₂ identical or different, preferably identical.

1.2.4. Monofunctional Fusion Proteins

Individually preferred examples thereof are:

[SYNZIP]-[O-RS]_{y} with y=1 or 2, preferably 1;

[SYNZIP]-[RNA-TP]ₓ with; x=1, 2, 3, 4, 5 or 6, preferably 2, 3 or 4;

As IC-TP and PSP is missing here, these may be preferably used in combination with an AFP molecule containing at least one C-TP and/or PSP segment.

1.3 Examples of Individual Fusion Proteins

Very specific examples of fusion protein of the inventions, and particular combinations thereof are listed below in Tables 1, 2 and 3. The content of this Tables 1, 2 and 3 also forms part of general disclosure of the specification and its content is not explicitly and literally repeated here in the general part. The disclosure of Tables 1 and 2 in the respective column designated “Fusion protein(s) comprising O-RS and RNA-TP segments” shall be considered as disclosed independently from the content of the other columns of Tables 1 and 2 referring to specific reports and host cell lines.

2. Functional Fragments and Mutants

Described herein are fragments and mutants of particular RNA-TPs, O-RSs, IC-TPs, PSPs, TNs, as well as SYNZIPs which are functional (i.e. have the RNA-binding activity of the parent RNA-TP, the targeting activity for intracellular structures of the parent IC-TP, the self-assembly activity of the parent PSP, the binding activity for RNA-TP of the parent TN, the enzymatic activity of the parent O-RS, or the heterodimeric coiled-coil formation ability of parent SYNZIPs, respectively). Such fragments and mutants can be characterized by a minimum degree of sequence identity as described herein. Said amino acid or nucleotide sequence identity means identity over the entire length of the thus characterized amino acid or nucleotide sequence, respectively. The percentage identity values can be determined as known in the art on the basis of BLAST alignments, blastp algorithms (protein-protein BLAST), or using the Clustal method (Higgins et al., Comput Appl. Biosci. 1989, 5(2):151-1).

Fragments and mutants of particular RNA-TPs, O-RSs, IC-TPs, SYNZIPS or PSPs which are useful in the present invention retain the relevant function (binding, self-assembly or enzymatic activity, respectively) of the parent polypeptide and can be obtained, e.g., by conservative amino acid substitution, i.e. the replacement of an amino acid residue with different amino acid residues having similar biochemical properties (e.g. charge, hydrophobicity and size) as known in the art. Typical examples are substitution of Leu by Ile or vice versa, substitution of Asp by Glu or vice versa, substitution of Asn by Gln or vice versa, and others.

3. Orthogonal Translation, tRNAs and POI Coding Sequences

The term “translation system” generally refers to a set of components necessary to incorporate a naturally occurring amino acid in a growing polypeptide chain (protein). Components of a translation system can include, e.g., ribosomes, tRNAs, aminoacyl tRNA synthetases, mRNA and the like. An aminoacyl tRNA synthetase (RS) is an enzyme capable of aminoacylating a tRNA with an amino acid or an amino acid analog. An RS used in processes of the invention is capable of aminoacylating a tRNA with the corresponding ncAA, i.e. aminoacylating a tRNA^{ncAA}. The term “orthogonal” as used herein refers to an element of a translation system (e.g., an orthogonal tRNA (O-tRNA) and/or an orthogonal aminoacyl tRNA synthetase (O-RS)) that is used with reduced efficiency by a translation system of interest (e.g., a cell). “Orthogonal” refers to the inability or reduced efficiency, e.g., less than 20% efficient, less than 10% efficient, less than 5% efficient, or e.g., less than 1% efficient, of an O-tRNA or an O-RS to function with the endogenous RS or endogenous tRNAs, respectively, of a translation system of interest. For example, an O-tRNA in a translation system of interest is aminoacylated by any endogenous RA of the translation system with reduced or even zero efficiency, when compared to aminoacylation of an endogenous tRNA by the endogenous RS. In another example, an O-RS aminoacylates any endogenous tRNA in the translation system of interest with reduced or even zero efficiency, as compared to aminoacylation of the endogenous tRNA by an endogenous RS. Specifically, the term “orthogonal translation system” or “OT system” is used herein to refer to a translation system using an O-RS/O-tRNA^{ncAA} pair that allows for introducing ncAA residues into a growing polypeptide chain.

O-RS/O-tRNA^{ncAA} pairs used in the invention preferably have following properties: the O-tRNA^{ncAA} is preferentially aminoacylated with the ncAA by the O-RS. In addition, the orthogonal pair functions in the translation system of interest (e.g, the cell) such that the O-tRNA^{ncAA} is used to incorporate the ncAA residue into the growing polypeptide chain of a POI. Incorporation occurs in a site specific manner. Specifically, the O-tRNA^{ncAA} recognizes a selector codon (e.g., an Amber, Ochre or Opal stop codon) in the mRNA coding for the POI.

The term “preferentially aminoacylates” refers to an efficiency of, e.g., about 50% efficient, about 70% efficient, about 75% efficient, about 85% efficient, about 90% efficient, about 95% efficient, or about 99% or more efficient, at which an O-RS aminoacylates an O-tRNA with an unnatural amino acid compared to an endogenous tRNA or amino acid of a translation system of interest (e.g., a cell). The unnatural amino acid is then incorporated into a growing polypeptide chain with high fidelity, e.g., at greater than about 75% efficiency for a given selector codon, at greater than about 80% efficiency for a given selector codon, at greater than about 90% efficiency for a given selector codon, at greater than about 95% efficiency for a given selector codon, or at greater than about 99% or more efficiency for a given selector codon.

tRNAs which can be used for being aminoacylated by a fusion protein of the present invention comprising at least one O-RS segment derived from a M. mazei pyrrolysyl tRNA synthetase include, but are not limited to pyrrolysyl tRNA of M. mazei and functional mutants thereof wherein the anticodon is the anticodon to a selector codon such as, e.g., the CUA anticodon to the Amber stop codon TAG, the anticodon UCA to the Opal stop codon TGA, and the anticodon UUA to the Ochre stop codon TAA. Examples for such pyrrolysyl tRNAs include, but are not limited to, those encoded by the nucleotide sequence of SEQ ID NO:4 (tRNA^{Pyl,CUA}), SEQ ID NO:5 (tRNA^{Pyl,UCA}) or SEQ ID NO:6 (tRNA^{Pyl,UUA}). Non-limiting examples of further suitable tRNAs are the following ones derived from pyrrolysyl tRNA of M. mazei:

tRNA^{pyl,CGA} Pyrrolysyl tRNA (for Serine codon), SEQ ID NO: 229

tRNA^{pyl,CGG} Pyrrolysyl tRNA (for Proline codon), SEQ ID NO: 230

tRNA^{pyl,UAA} Pyrrolysyl tRNA (for Leucine codon), SEQ ID NO: 231

tRNA^{pyl,UAG} Pyrrolysyl tRNA (for Leucine codon), SEQ ID NO: 232

tRNA^{pyl,CCG} Pyrrolysyl tRNA (for Arginine codon), SEQ ID NO: 233

tRNA^{pyl,AUA} Pyrrolysyl tRNA (for Isoleucine codon), SEQ ID NO: 234

The term “selector codon” as used herein refers to a codon that is recognized (i.e. bound) by the O-tRNA^{ncAA} in the translation process. The term is also used for the corresponding codons in polypeptide-encoding sequences of polynucleotides which are not messenger RNAs (mRNAs), e.g. DNA plasmids. The new OT systems described herein allow for orthogonal translation of POIs in a manner that is selective for the mRNA of said POIs compared to other mRNAs present in the cytoplasm of the cell. Nevertheless, it is preferable that the selector codon is a codon of low abundance in the cell chosen for expression, for example a codon of low abundance in naturally occurring eukaryotic cells. The new OT systems bring the mRNA of the POIs, the O-RS and the tRNA^{ncAA} into proximity to one another, thus supporting the introduction of the ncAA (rather than the introduction of an amino acid of a different tRNA that might potentially bind to the selector codon) at the selector codon-encoded amino acid position of the POI. Thus, the selector codon can be a sense codon. Nevertheless, in preferred embodiments, the selector codon is a codon that is not recognized by endogenous tRNAs of the cell used for preparing the POI.

The anticodon of the O-tRNA^{ncAA} binds to a selector codon within an mRNA (the mRNA of the POI) and thus incorporates the ncAA site-specifically into the growing chain of the polypeptide (POI) encoded by said mRNA. Examples for selector codons which are useful in the new OT systems described herein include, but are not limited to:
nonsense codons, such as stop codons, e.g., Amber (UAG), Ochre (UAA), and Opal (UGA) codons;
codons consisting of more than three bases (e.g., four base codons);
codons derived from natural or unnatural base pairs; and
sense codons.

Where a selector codon is used that is a sense codon (i.e., a natural three base codon), it is preferable that the endogenous translation system of the cell used for POI expression according to a method of the present invention does not (or only scarcely) use said natural three base codon, e.g., a cell that is lacking, or has a reduced abundance of, a tRNA that recognizes the natural three base codon or a cell wherein the natural three base codon is a rare codon. The use of one or more stop codons, such as one or more of Amber, Ochre and Opal, as selector codons in the present invention is particularly preferred.

A number of selector codons can be introduced into a polynucleotide encoding a desired polypeptide (target polypeptide, POI), e.g., one or more, two or more, more than three, etc. selector codons. A POI can carry two or more ncAA residues. Said ncAA residues can be the same and encoded by the same type of selector codon, or can be different and encoded by different selector codons.

An anticodon has the reverse complement sequence of the corresponding codon.

A suppressor tRNA is a tRNA (such as an O-tRNA^{ncAA}) that alters the reading of a messenger RNA (mRNA) in a given translation system (e.g., a cell). A suppressor tRNA can read through, e.g., a stop codon, a four base codon, or a rare codon.

The O-tRNA is preferentially aminoacylated by O-RS (rather than endogenous synthetases) and is capable of decoding a selector codon, as described herein. The O-RS recognizes the O-tRNA, e.g., with an extended anticodon loop, and preferentially aminoacylates the O-tRNA with an ncAA.

The O-tRNA and the O-RS used in the methods and/or fusion proteins of the invention can be naturally occurring or can be derived by mutation of a naturally occurring tRNA and/or RS from a variety of organisms. In various embodiments, the tRNA and RS are derived from at least one organism. In another embodiment, the tRNA is derived from a naturally occurring or mutated naturally occurring tRNA from a first organism and the RS is derived from naturally occurring or mutated naturally occurring RS from a second organism.

A suitable (orthogonal) tRNA/RS pair may be selected from libraries of mutant tRNA and RS, e.g. based on the results of a library screening. Alternatively, a suitable tRNA/RS pair may be a heterologous tRNA/synthetase pair that is imported from a source species into the translation system. Preferably, the cell used as translation system is different from said source species. Methods for evolving tRNA/RS pairs are described, e.g., in WO 02/085923 and WO 02/06075.

Conventional site-directed mutagenesis can be used to introduce selector codons into the coding sequence of a POI.

4. Nucleic Acid Molecules

The invention also relates to nucleic acid molecules (single-stranded or double-stranded DNA and RNA sequences, for example cDNA, mRNA), or combinations of such nucleic acid molecules, comprising a nucleotide sequence that encodes for at least one of the fusion proteins of the present invention, and/or a nucleotide sequence complementary thereto.

Further, the invention relates to nucleic acid molecules (single-stranded or double-stranded DNA and RNA sequences, for example cDNA, mRNA), or combinations of such nucleic acid molecules, comprising (i) a nucleotide sequences (CS^{POI}) that encodes at least one POI, said POI comprising one or more ncAA residues which are encoded in the CS^{POI} by selector codons, and (ii) a targeting nucleotide sequence (TN) as described herein, wherein an RNA molecule comprising (the RNA version of) said TN is able to interact via said TN with an RNA-targeting polypeptide (RNA-TP).

The nucleic acid molecules of the invention can in addition contain untranslated sequences of the 3′- and/or 5′-end of the coding gene region. The TN is preferably located at the 3′ end of the nucleic acid molecule encoding the POI(s). For example, nucleic acid molecules of the invention encoding the POI(s) can be prepared by introducing at least one TN at (in particular 3′ of) the 3′ untranslated region using common cloning techniques known in the art.

The nucleic acid molecules of the invention can in addition contain untranslated sequences of the 3′- and/or 5-end of the coding gene region.

The invention further relates to, in particular recombinant, expression constructs or expression cassettes, containing, under the genetic control of regulatory nucleic acid sequences the nucleic acid sequence of the nucleic acid molecule, or combination of nucleic acid molecules, of the invention as described herein. The expression cassettes of the invention thus comprise the nucleic acid sequence coding for at least one POI (plus TN) or at least one fusion protein of the invention, and/or a nucleic acid sequence complementary thereto. The invention also relates to, in particular recombinant, vectors, comprising at least one of these expression constructs (expression vectors).

An expression cassette typically comprises a promoter sequence that is located 5′ (upstream) of, and functionally linked with, the nucleic acid sequence encoding the to-be-expressed POI(s) or fusion protein(s), a terminator sequence 3′ (downstream) of said encoding sequence and optionally further regulatory elements. Examples of such further regulatory elements include, but are not limited to, targeting sequences, enhancers, polyadenylation signals, selectable markers, amplification signals, replication origins and the like. Suitable regulatory sequences are described for example in Goeddel, Gene Expression Technology: Methods in Enzymology 185, Academic Press, San Diego, Calif. (1990).

In addition to these regulatory sequences, the natural regulation of these sequences can still be present before the actual structural genes and optionally can have been genetically altered, so that the natural regulation has been switched off and expression of the genes has been increased. The nucleic acid construct can, however, also be of simpler construction, i.e. no additional regulatory signals have been inserted before the coding sequence and the natural promoter, with its regulation, has not been removed. Instead, the natural regulatory sequence is mutated so that regulation no longer takes place and gene expression is increased.

A “functional” linkage of elements of nucleic acid molecules, such as promotor, polypeptide-encoding sequence, terminator, regulators, means that these elements are arranged such that the encoding sequence can be transcribed and the optional regulatory elements can perform their regulation of said transcription. This can be achieved by a direct linkage of the elements in one and the same nucleic acid molecule. However, such direct linkage is not necessarily required. Genetic control sequences, for example enhancer sequences, can even exert their function on the target sequence from more remote positions or even from other DNA molecules. Arrangements are preferred in which the nucleic acid sequence to be transcribed is positioned downstream (i.e. at the 3′-end of) the promoter sequence, so that the two sequences are joined together covalently. The distance between the promoter sequence and the nucleic acid sequence to be expressed can be smaller than 200 base pairs, or smaller than 100 base pairs or smaller than 50 base pairs.

For expression in a cell, the expression cassette is advantageously inserted into an expression vector. Expression vectors are chosen according to the cell to be used for expression which makes optimal expression of the encoding nucleotide sequences in the cell possible. Vectors are well known by a person skilled in the art and are given for example in “Cloning vectors” (Pouwels P. H. et al., Ed., Elsevier, Amsterdam-New York-Oxford, 1985). Examples of expression vectors include, but are not limited to, plasmids, viral vectors (phages), e.g. SV40, CMV, baculovirus and adenovirus, transposons, IS elements, phasmids, cosmids, and linear or circular DNA. See, e.g., the book “Cloning Vectors” (Eds. Pouwels P. H. et al. Elsevier, Amsterdam-New York-Oxford, 1985, ISBN 0 444 904018). These vectors can be replicated autonomously in the (host) cell or can be replicated chromosomally. Expression vectors comprising at least one expression cassette of the present invention represent a further aspect of the invention.

For the expression of a POI in a cell according to the present invention, it is possible, e.g., to introduce a nucleic acid molecule which encodes the POI (e.g. an expression vector of the invention) into the cell. Alternatively, an existing gene of the cell can be modified so as to comprise selector codons at those amino acid positions where the POI is intended to carry ncAA residues. Methods for introducing (recombinant) polypeptide-encoding nucleic acid molecules into, or for modifying existing genes of, a cell are known in the art.

The term “expression” describes, in the context of the invention, the production of polypeptides encoded by the corresponding nucleic acid sequence in a cell. The term “expression” is also used for the production of tRNA molecules encoded by nucleic acid sequences in the cell.

The nucleic acid molecules of the invention, including the expression cassettes and expression vectors of the invention can be prepared using common cloning techniques known in the art. Common recombination and cloning techniques are used, as described for example in T. Maniatis, E. F. Fritsch and J. Sambrook, Molecular Cloning: A Laboratory Manual, Cold Spring Harbor Laboratory, Cold Spring Harbor, N.Y. (1989) and in T. J. Silhavy, M. L. Berman and L. W. Enquist, Experiments with Gene Fusions, Cold Spring Harbor Laboratory, Cold Spring Harbor, N.Y. (1984) and in Ausubel, F. M. et al., Current Protocols in Molecular Biology, Greene Publishing Assoc. and Wiley Interscience (1987).

The nucleic acid molecules, or combinations of nucleic acid molecules, of the invention, including expression cassettes and expression vectors of the invention, can be isolated, for example by methods known in the art.

An “isolated” nucleic acid molecule is separated from other nucleic acid molecules that are present in the natural source of the nucleic acid, and moreover can be essentially free of other cellular material or culture medium, when it is produced by recombinant techniques, or free of chemical precursors or other chemicals, when it is chemically synthesized.

A nucleic acid molecule according to the invention can be isolated by standard techniques of molecular biology and the sequence information provided according to the invention. For example, cDNA can be isolated from a suitable cDNA-bank, using one of the concretely disclosed complete sequences or a segment thereof as hybridization probe and standard hybridization techniques (as described for example in Sambrook, J., Fritsch, E. F. and Maniatis, T. Molecular Cloning: A Laboratory Manual. 2nd edition, Cold Spring Harbor Laboratory, Cold Spring Harbor Laboratory Press, Cold Spring Harbor, N.Y., 1989). Moreover, a nucleic acid molecule, comprising one of the disclosed sequences or a segment thereof, can be isolated by polymerase chain reaction, using the oligonucleotide primers that were constructed on the basis of this sequence. The nucleic acid thus amplified can be cloned into a suitable vector and can be characterized by DNA sequence analysis. The oligonucleotides according to the invention can moreover be produced by standard methods of synthesis, e.g. with an automatic DNA synthesizer.

5. ncAAs and Post-Translational POI Modifications

The abbreviation “ncAA” refers generally to any non-canonical or non-natural amino acid, or amino acid residue, that is not among the 22 naturally occurring proteinogenic amino acids. Numerous ncAAs are well known in the art (see, e.g., Liu et al., Annu Rev Biochem 2010, 79:413-444; Lemke, ChemBioChem 2014, 15:1691-1694). The term “ncAA” also refers to amino acid derivatives, for example α-hydroxy acids (rather than α-amino acids). Such derivatives have been shown to be translationally incorporable as well. See, e.g., Ohta et al., 2008, ChemBioChem 9:2773-2778. Accordingly, the meaning of terms such as “aminoacylate” or “aminoacylation” used herein is not limited to the RS-catalyzed linkage of a tRNA and an α-amino acid but also includes the RS-catalyzed linkage of a tRNA and a ncAA derivative such as an α-hydroxy acid.

Particular preferred ncAAs for use in the present invention are those which can be post-translationally further modified, for example using click chemistry reactions. Such click reactions include strain-promoted inverse-electron-demand Diels-Alder cycloadditions (SPIEDAC; see, e.g., Devaraj et al., Angew Chem Int Ed Engl 2009, 48:7013)) as well as cycloadditions between strained cycloalkynyl groups, or strained cycloalkynyl analog groups having one or more of the ring atoms not bound by the triple bond substituted by amino groups), with azides, nitrile oxides, nitrones and diazocarbonyl reagents (see, e.g., Sanders et al., J Am Chem Soc 2010, 133:949; Agard et al., J Am Chem Soc 2004, 126:15046), for example strain promoted alkyne-azide cycloadditions (SPAAC). Such click reactions allow for ultrafast and biorthogonal covalent site-specific coupling of ncAA-labeling groups of target polypeptides with suitable groups of coupling partner molecule. Pairs of docking and labeling groups which can react via the above-mentioned click reactions are known in the art. Examples of suitable ncAAs for use in the present invention comprising docking groups include, but are not limited to, the ncAAs (“unnatural amino acids”, “UAAs”) described, e.g., in WO 2012/104422 and WO 2015/107064. Optionally substituted strained alkynyl groups include, but are not limited to, optionally substituted trans-cyclooctenyl groups, such as those described in. Optionally substituted strained alkenyl groups include, but are not limited to, optionally substituted cyclooctynyl groups, such as those described in WO 2012/104422 and WO 2015/107064. Optionally substituted tetrazinyl groups include, but are not limited to, those described in WO 2012/104422 and WO 2015/107064.

The ncAAs used in the context of the present invention can be used in the form of their salt. Salts of an ncAA as described herein means acid or base addition salts, especially addition salts with physiologically tolerated acids or bases. Physiologically tolerated acid addition salts can be formed by treatment of the base form of an ncAA with appropriate organic or inorganic acids. ncAAs containing an acidic proton may be converted into their non-toxic metal or amine addition salt forms by treatment with appropriate organic and inorganic bases. Salts of carboxyl groups of ncAAs can be produced in a manner known in the art and comprise inorganic salts, for example sodium, calcium, ammonium, iron and zinc salts, and salts with organic bases, for example amines, such as triethanolamine, arginine, lysine, piperidine, etc. ncAAs may also be used in the form of salts of acid addition, for example salts with mineral acids, such as hydrochloric acid or sulfuric acid and salts with organic acids, such as acetic acid and oxalic acid. The ncAAs and salts thereof which are useful in the present invention also comprise the hydrates and solvent addition forms thereof, e.g. hydrates, alcoholates and the like.

Physiologically tolerated acids or bases are in particular those which are tolerated by the translation system used for preparation of POI with ncAA residues, e.g. are substantially non-toxic to living eukaryotic cells.

ncAAs, and salts thereof, useful in the context of the present the invention can be prepared by analogy to methods which are well known in the art and are described, e.g., in the various publications cited herein.

The nature of the coupling partner molecule depends on the intended use. For example, the target polypeptide may be coupled to a molecule suitable for imaging methods or may be functionalized by coupling to a bioactive molecule. For instance, in addition to the docking group, a coupling partner molecule may comprise a group selected from, but not limited to, dyes (e.g. fluorescent, luminescent, or phosphorescent dyes, such as dansyl, coumarin, fluorescein, acridine, rhodamine, silicon-rhodamine, BODIPY, or cyanine dyes), molecules able to emit fluorescence upon contact with a reagent, chromophores (e.g., phytochrome, phycobilin, bilirubin, etc.), radiolabels (e.g. radioactive forms of hydrogen, fluorine, carbon, phosphorous, sulphur, or iodine, such as tritium, ¹⁸F, ¹¹C, ¹⁴C, ³²P, ³³P, ³³S, ³⁵S, ¹¹In, ¹²⁵I, ¹²³I, ¹³¹I, ²¹²B, ⁹⁰Y or ¹⁸⁶Rh), MRI-sensitive spin labels, affinity tags (e.g. biotin, His-tag, Flag-tag, strep-tag, sugars, lipids, sterols, PEG-linkers, benzylguanines, benzylcytosines, or co-factors), polyethylene glycol groups (e.g., a branched PEG, a linear PEG, PEGs of different molecular weights, etc.), photocrosslinkers (such as p-azidoiodoacetanilide), NMR probes, X-ray probes, pH probes, IR probes, resins, solid supports and bioactive compounds (e.g. synthetic drugs). Suitable bioactive compounds include, but are not limited to, cytotoxic compounds (e.g., cancer chemotherapeutic compounds), antiviral compounds, biological response modifiers (e.g., hormones, chemokines, cytokines, interleukins, etc.), microtubule affecting agents, hormone modulators, and steroidal compounds. Specific examples of useful coupling partner molecules include, but are not limited to, a member of a receptor/ligand pair; a member of an antibody/antigen pair; a member of a lectin/carbohydrate pair; a member of an enzyme/substrate pair; biotin/avidin; biotin/streptavidin and digoxin/antidigoxin.

The ability of certain (labeling groups of) ncAA residues to be coupled covalently in situ to (the docking groups of) conjugation partner molecules, in particular by a click reaction as described herein, can be used for detecting a target polypeptide having such ncAA residue(s) within a eukaryotic cell or tissue expressing the target polypeptide, and for studying the distribution and fate of the target polypeptides. Specifically, the method of the present invention for preparing a POI by expression in (e.g., eukaryotic) cells can be combined with super-resolution microscopy (SRM) to detect the POI within the cell or a tissue of such cells. Several SRM methods are known in the art and can be adapted so as to utilize click chemistry for detecting a target polypeptide expressed by a eukaryotic cell of the present invention. Specific examples of such SRM methods include DNA-PAINT (DNA point accumulation for imaging in nanoscale topography; described, e.g., by Jungmann et al., Nat Methods 11:313-318, 2014), dSTORM (direct stochastic optical reconstruction microscopy) and STED (stimulated emission depletion) microscopy.

6. Translational Preparation of POIs in Cells The OT systems provided by the invention allow for the translational preparation of a POI in a cell.

The cell used for preparing a POI according to the invention can be a prokaryotic cell. Alternatively, the cell used for preparing a POI according to the invention can be a eukaryotic cell. The cell used for preparing a POI according to the invention can be a separate cell such as, e.g., a single-cell microorganism or a cell line derived from cells of multicellular organisms. Alternatively, the cell used for preparing a POI according to the invention can be present in (and part of) a tissue, an organ, a body part or an entire multicellular organism. Thus, the methods of the invention for preparing a POI can be performed with a separate cell or a cell culture, or with a tissue or tissue culture, organ, body part or (entire multicellular) organism.

Eukaryotic cells are often more difficult to handle and manipulate compared to prokaryotes such as, e.g., E. coli, and therefore not or only very difficult accessible to known approaches for POI-selective orthogonal translation such as those described in the “Background of the invention” section above. The OT system and the methods of the invention are therefore particular advantageous when use for POI expression in eukaryotic cells (including, e.g., single- and multicellular eukaryotic organisms, and eukaryotic cell lines).

In principle, all prokaryotic or eukaryotic cells can be used for preparing a POI according to a method of the present invention. Microorganisms such as, e.g., bacteria, fungi or yeasts can be used, as well as eukaryotic cells, such as, e.g., mammalian cells, insect cells, yeast cells and plant cells. Eukaryotic cells and in particular mammalian cells are particularly preferred.

The cell used for preparing a POI according to the invention carries a POI-encoding nucleotide sequence (CS^{POI}) wherein the ncAA residue(s) of the POI are encoded by selector codon(s). Said CS^{POI} is functionally linked with one or more targeting sequences (TNs). Translation yields an mRNA comprising the CS^{POI} and the TN(s). The cell further comprises one or more fusion proteins of the present invention, wherein said fusion protein(s) comprise at least one O-RS segment and at least one RNA-TP segment. Said O-RS and RNA-TP can be on separate fusion proteins (e.g. AFPs) of the invention. Alternatively, said O-RS and RNA-TP can be on one and the same fusion protein (e.g. on an RNA-TP/O-RS fusion protein or an AFP) of the invention. Via (at least one of) its TN(s) said mRNA can interact with (bind to) at least one of the RNA-TP segments of the fusion proteins of the invention in the cell.

The cell further comprises one or more orthogonal tRNA^{ncAA} molecules (O-tRNA^{ncAA}) which carry the anticodon(s) to the selector codon(s) of the CS^{POI}. Said O-tRNA^{ncAA} molecules and one or more of the O-RS segments of the fusion proteins in the cell form one or more orthogonal O-RS/O-tRNA^{ncAA} pairs which allow for introducing the ncAA residue(s) into the amino acid sequence of the (translationally prepared) POI.

The interaction of the mRNA comprising CS^{POI} and TN(s) with the RNA-TP segment(s), the aminoacylation of the O-tRNA^{ncAA} with the ncAAs by the O-RS segment(s), and the translational preparation of the POI including the introduction of the ncAA residue(s) thought to take place in the cytoplasm, more particularly in the OT assembly (OT organelle), of the cell in the presence of the ncAAs.

The mRNA comprising CS^{POI} and TN(s) (mRNA^{POI}) can be generated from a recombinant construct (e.g. expression vector) introduced into the cell. Alternatively, one or more endogenous genes of the cell can be modified so as to comprise one or more selector codons and one or more TNs. Techniques for introducing recombinant constructs into a cell as well as methods for modifying endogenous genes of a cell are well known in the art.

The tRNA^{ncAA} molecules and fusion proteins of the invention can be generated from a recombinant construct (e.g. expression vector) introduced into the cell.

Using expression vectors according to the invention, recombinant cells can be produced which can be used for preparing a POI using a method of the present invention. Advantageously, the recombinant vectors according to the invention, described above, are introduced into a suitable cell and expressed.

The cell used for preparing a POI as described herein can be prepared by introducing nucleotide sequences encoding the fusion protein(s), the tRNA^{ncAA} molecule(s) and the POI into the cell. Said nucleotide sequences can be located on separate nucleic acid molecules (vectors) or on the same nucleic acid molecule (e.g., vector), in any combination, and can be introduced into the cell in combination or sequentially.

Preferably common cloning and transfection techniques, known by a person skilled in the art, are used, for example co-precipitation, protoplast fusion, electroporation, virus-mediated gene delivery, lipofection, microinjection or others, for introducing the stated nucleic acid molecules in the respective cell. Suitable techniques are described for example in Current Protocols in Molecular Biology, F. Ausubel et al., Ed., Wiley Interscience, New York 1997, or Sambrook et al. Molecular Cloning: A Laboratory Manual. 2^{nd} edition, Cold Spring Harbor Laboratory, Cold Spring Harbor Laboratory Press, Cold Spring Harbor, N.Y., 1989.

For the methods of the present invention, the cell used for POI expression is grown or cultured in a manner known by a person skilled in the art. Depending on the type of cell, a liquid medium can be used for culturing. Culture can be batchwise, semi-batchwise or continuous. Nutrients can be present at the beginning of the culturing or can be supplied later, semi-continuously or continuously.

The expressed POIs can be purified by known techniques, such as, e.g., molecular sieve chromatography (gel filtration), such as Q-sepharose chromatography, ion exchange chromatography and hydrophobic chromatography, and other common protein purification techniques such as ultrafiltration, crystallization, salting-out, dialysis and native gel electrophoresis. Suitable methods are described, for example, in Cooper, T. G., Biochemische Arbeitsmethoden [Biochemical processes], Verlag Walter de Gruyter, Berlin, New York or in Scopes, R., Protein Purification, Springer Verlag, New York, Heidelberg, Berlin.

For isolating a POI, it can be advantageous to link the POI with a tag that can serve for easier purification. This can be achieved by introducing a corresponding tag-encoding sequence into the CSPOI. Suitable tags for protein purification are well known in the art and include, e.g., histidine tags (e.g., His6 tag (SEQ ID NO: 685)) and epitopes that can be recognized as antigens of antibodies (described for example in Harlow, E. and Lane, D., 1988, Antibodies: A Laboratory Manual. Cold Spring Harbor (N.Y.) Press). These tags can serve for attaching the proteins to a solid carrier, for example a polymer matrix, which can for example be used as packing in a chromatography column, or can be used on a microtiter plate or on some other carrier.

A tag linked to a POI can also serve for detecting the POI. Tags for protein detection are well known in the art and include, e.g., fluorescent dyes, enzyme markers, which form a detectable reaction product after reaction with a substrate, and others.

For preparing a POI according to a method of the present invention, the expression can be achieved by culturing the cell in the presence of one or more ncAAs corresponding to the ncAA residue(s) of the POI (wherein said ncAAs may expediently be comprised in the culture medium) for a time suitable to allow translation of the POI. Depending on the nucleic acid(s) encoding the POI (and optionally the fusion proteins of the invention and/or the tRNA^{ncAA} molecules), it may be required to induce expression by adding a compound inducing transcription, such as, e.g., arabinose, isopropyl #-D-thiogalactoside (IPTG) or tetracycline that allows transcription.

After translation, the POI may optionally be recovered from the translation system. For this purpose, the POI can be recovered and purified, either partially or substantially to homogeneity, according to procedures known to and used by those of skill in the art. Unless the target polypeptide is secreted into the culture medium, recovery usually requires cell disruption. Methods of cell disruption are well known in the art and include physical disruption, e.g., by (ultrasound) sonication, liquid-sheer disruption (e.g., via French press), mechanical methods (such as those utilizing blenders or grinders) or freeze-thaw cycling, as well as chemical lysis using agents which disrupt lipid-lipid, protein-protein and/or protein-lipid interactions (such as detergents), and combinations of physical disruption techniques and chemical lysis. Standard procedures for purifying polypeptides from cell lysates or culture media are also well known in the art and include, e.g., ammonium sulfate or ethanol precipitation, acid or base extraction, column chromatography, affinity column chromatography, anion or cation exchange chromatography, phosphocellulose chromatography, hydrophobic interaction chromatography, hydroxylapatite chromatography, lectin chromatography, gel electrophoresis and the like. Protein refolding steps can be used, as desired, in making correctly folded mature proteins. High performance liquid chromatography (HPLC), affinity chromatography or other suitable methods can be employed in final purification steps where high purity is desired. Antibodies made against the polypeptides of the invention can be used as purification reagents, i.e. for affinity-based purification of the polypeptides. A variety of purification/protein folding methods are well known in the art, including, e.g., those set forth in Scopes, Protein Purification, Springer, Berlin (1993); and Deutscher, Methods in Enzymology Vol. 182: Guide to Protein Purification, Academic Press (1990); and the references cited therein.

As noted, those of skill in the art will recognize that, after synthesis, expression and/or purification, polypeptides can possess a conformation different from the desired conformations of the relevant polypeptides. For example, polypeptides produced by prokaryotic systems often are optimized by exposure to chaotropic agents to achieve proper folding. During purification from, e.g., cell lysates, the expressed polypeptide is optionally denatured and then renatured. This is accomplished, e.g., by solubilizing the proteins in a chaotropic agent such as guanidine HCl. In general, it is occasionally desirable to denature and reduce expressed polypeptides and then to cause the polypeptides to re-fold into the preferred conformation. For example, guanidine, urea, DTT, DTE, and/or a chaperonin can be added to a translation product of interest. Methods of reducing, denaturing and renaturing proteins are well known to those of skill in the art. Polypeptides can be refolded in a redox buffer containing, e.g., oxidized glutathione and L-arginine.

Also described are polypeptides produced by the methods of the invention. Such polypeptides can be prepared by a method of the invention that makes use of the OT system described herein.

7. Kits

The present invention also provides kits for preparing a POI having at least one non-canonical amino acid (ncAA) residue. The kit of the invention may comprise at least one expression vector for at least one fusion protein of the present invention. The fusion protein(s) encoded by the expression vector(s) in the kit may comprise at least one O-RS segment and at least one RNA-TP segment. The kit may further comprise at least one ncAA, or salt thereof, corresponding to the at least one ncAA residue of the POI. Expediently said O-RS segment is capable of aminoacylating a tRNA with the at least one ncAA. The kit may further comprise at least one expression vector for an orthogonal tRNA^{ncAA} (O-tRNA^{ncAA}) molecule. Further components of the kit may include at least one expression vector comprising a multiple cloning site and a targeting nucleotide sequence (TN), wherein an RNA molecule comprising said TN is able to interact via said TN with an RNA-targeting polypeptide (RNA-TP). Expediently said TN is a sequence, which, when present in an RNA molecule, is able to interact with an RNA-TP segment of at least one of the fusion protein(s) encoded by the expression vector(s) comprised by the kit. The kit may further comprise at least one reporter construct encoding an easily detectable (e.g. fluorescent) reporter polypeptide having at least one non-canonical amino acid (ncAA) residue such that the mRNA translated from said construct comprises a TN as described herein.

The kits of the present invention can be used in methods of the invention for preparing ncAA-residue containing POIs as described herein.

### PARTICULAR EMBODIMENTS

The present invention further provides the following non-limiting embodiments E1 to E50.
E1: An assembler fusion protein (AFP) comprising:
   (a) at least one first polypeptide segment acting as assembler (AP) that is selected from:
      (a1) a polypeptide segment derived from an intracellular targeting polypeptide (IC-TP segment), wherein said intracellular targeting polypeptide targets, and thus becomes locally enriched at, an intracellular structural element within or directly adjacent to the cytoplasm; and
      (a2) a polypeptide segment derived from a phase separation polypeptide (PSP segment), wherein said phase separation polypeptide has the ability to undergo self-association in the cytoplasm of a cell so as to create sites of high local concentration in the cytoplasm, and
   (b) at least one second polypeptide segment acting as an effector (EP) that is selected from:
      b1) an RNA-targeting polypeptide (RNA-TP) segment, and
      b2) an orthogonal aminoacyl tRNA synthetase (O-RS) segment;
   wherein said polypeptide segments are functionally linked in said AFP.
E2: The AFP of E1 comprising at least two APs, preferably at least one IC-TP segment and at least one PSP segment.
E3: The AFP of E1 or E2 having one of the following structures (from the N-terminus to the C-terminus):
   [IC-TP]ₘ-[EP]ₒ
   [EP]ₒ-[IC-TP]ₘ
   [PSP]ₙ-[EP]ₒ
   [EP]ₒ-[PSP]ₙ
   [IC-TP]ₘ-[EP]ₒ-[PSP]ₙ
   [PSP]ₙ-[EP]ₒ-[IC-TP]ₘ
   [IC-TP]ₘ-[PSP]ₙ-[EP]ₒ
   [EP]ₒ-[PSP]ₙ-[IC-TP]ₘ
   [PSP]ₙ-[IC-TP]ₘ-[EP]ₒ
   [EP]ₒ-[IC-TP]ₘ-[PSP]ₙ wherein m, n and o, independently of each other, are integers selected from 1, 2, 3, 4
   or 5, and “-” designates a peptidic linkage.
E4: The AFP of any one of E1-E3, wherein the at least one EP is selected from RNA-TP segments.
E5: The AFP of any one of E1-E3, wherein the at least one EP is selected from O-RS segments.
E6: The AFP of any one of E1-E3 comprising at least one EP selected from RNA-TP segments and at least one EP selected from O-RS segments.
E7: The AFP of any one of E1-E6 comprising at least one IC-TP segment selected from dyneins and kinesins, and fragments and mutants of dyneins and kinesins, which retain the ability to target, and become enriched at, the plus or the minus end of microtubules.
E8: The AFP of any one of E1-E6 comprising at least one IC-TP segment selected from transmembrane domains of membrane proteins, and functional fragments and mutants of transmembrane domains which retain the ability to target, and become enriched at, the cytoplasmic side of membranes, in particular membranes selected from the cell membrane, nuclear membrane and mitochondrial membrane.
E9: The AFP of any one of E1-E8 comprising at least one IC-TP segment selected from:
   KIF16B₁₋₄₀₀ comprising the amino acid sequence of SEQ ID NO:20, or a functional fragment or mutant thereof having at least 60%, at least 70%, at least 80%, at least 90%, at least 95%, at least 96%, at least 97%, at least 98% or at least 99% amino acid sequence identity to the amino acid sequence of SEQ ID NO:20;
   KIF13A_{1-411,Δ390} comprising the amino acid sequence of SEQ ID NO:22, or a functional fragment or mutant thereof having at least 60%, at least 70%, at least 80%, at least 90%, at least 95%, at least 96%, at least 97%, at least 98% or at least 99% amino acid sequence identity to the amino acid sequence of SEQ ID NO:22;
   TOMM20₁₋₇₀ comprising the amino acid sequence of SEQ ID NO:24, or a functional fragment or mutant thereof having at least 60%, at least 70%, at least 80%, at least 90%, at least 95%, at least 96%, at least 97%, at least 98% or at least 99% amino acid sequence identity to the amino acid sequence of SEQ ID NO:24;
   LcK comprising the amino acid sequence of SEQ ID NO:26, or a functional fragment or mutant thereof having at least 60%, at least 70%, at least 80%, at least 90%, at least 95%, at least 96%, at least 97%, at least 98% or at least 99% amino acid sequence identity to the amino acid sequence of SEQ ID NO:26;
   FRB-CD28 comprising the amino acid sequence of SEQ ID NO:28, or a functional fragment or mutant thereof having at least 60%, at least 70%, at least 80%, at least 90%, at least 95%, at least 96%, at least 97%, at least 98% or at least 99% amino acid sequence identity to the amino acid sequence of SEQ ID NO:28;
   FUS-CD28 comprising the amino acid sequence of SEQ ID NO:30, or a functional fragment or mutant thereof having at least 60%, at least 70%, at least 80%, at least 90%, at least 95%, at least 96%, at least 97%, at least 98% or at least 99% amino acid sequence identity to the amino acid sequence of SEQ ID NO:30;
   EB1 comprising the amino acid sequence of SEQ ID NO:302, or a functional fragment or mutant thereof having at least 60%, at least 70%, at least 80%, at least 90%, at least 95%, at least 96%, at least 97%, at least 98% or at least 99% amino acid sequence identity to the amino acid sequence of SEQ ID NO:303
   CG1 comprising the amino acid sequence of SEQ ID NO:304, or a functional fragment or mutant thereof having at least 60%, at least 70%, at least 80%, at least 90%, at least 95%, at least 96%, at least 97%, at least 98% or at least 99% amino acid sequence identity to the amino acid sequence of SEQ ID NO:304
   EBAG9 comprising the amino acid sequence of SEQ ID NO:292 (full length) or a functional fragment or mutant thereof having at least 60%, at least 70%, at least 80%, at least 90%, at least 95%, at least 96%, at least 97%, at least 98% or at least 99% amino acid sequence identity to SEQ ID NO:292; or comprising the first 29 N-terminal amino acid residues of SEQ ID NO:294; or a functional fragment or mutant thereof having at least 60%, at least 70%, at least 80%, at least 90%, at least 95%, at least 96%, at least 97%, at least 98% or at least 99% amino acid sequence identity to SEQ ID NO:294
   CMP Sia Tr, comprising the amino acid sequence of SEQ ID NO:296, or a functional fragment or mutant thereof having at least 60%, at least 70%, at least 80%, at least 90%, at least 95%, at least 96%, at least 97%, at least 98% or at least 99% amino acid sequence identity to the amino acid sequence of SEQ ID NO:296; and
   P450 2C1 targeting the cytoplasmic side of the ER membrane or a functional fragment or mutant thereof having at least 60%, at least 70%, at least 80%, at least 90%, at least 95%, at least 96%, at least 97%, at least 98% or at least 99% amino acid sequence identity thereto, in particular a fragment comprising the N-terminal first 27 (SEQ ID NO:298); or first 29 (SEQ ID NO:300) amino acid residues; or a functional fragment or mutant thereof having at least 60%, at least 70%, at least 80%, at least 90%, at least 95%, at least 96%, at least 97%, at least 98% or at least 99% amino acid sequence identity to SEQ ID NO:298 or 300.
E10: The AFP of any one of E1-E9 comprising at least one PSP segment selected from intrinsically disordered proteins (IDPs), in particular prion-like domains, and functional fragments and mutants of IDPs, or prio-like domains, which retain the ability to undergo self-association in the cytoplasm of a cell so as to create sites of high local concentration in the cytoplasm.
E11: The AFP of any one of E1-E10 comprising at least one PSP segment selected from:
   SPD5 comprising the amino acid sequence of SEQ ID NO:32, or a functional fragment or mutant thereof having at least 60%, at least 70%, at least 80%, at least 90%, at least 95%, at least 96%, at least 97%, at least 98% or at least 99% amino acid sequence identity to the amino acid sequence of SEQ ID NO:32;
   FUS comprising the amino acid sequence of SEQ ID NO:34, or a functional fragment or mutant thereof having at least 60%, at least 70%, at least 80%, at least 90%, at least 95%, at least 96%, at least 97%, at least 98% or at least 99% amino acid sequence identity to the amino acid sequence of SEQ ID NO:34; and
   EWSR1 comprising the amino acid sequence of SEQ ID NO:36, or a functional fragment or mutant thereof having at least 60%, at least 70%, at least 80%, at least 90%, at least 95%, at least 96%, at least 97%, at least 98% or at least 99% amino acid sequence identity to the amino acid sequence of SEQ ID NO:36.
E12: The AFP of any one of E1-E11 comprising at least one RNA-TP segment selected from RNA-binding segments of viral coat proteins, and functional fragments and mutants of RNA-binding segments of viral coat proteins which retain the ability to interact specifically with an RNA motif of the virus.
E13: The AFP of any one of E1-E12 comprising at least one RNA-TP segment selected from:
   MCP comprising the amino acid sequence of SEQ ID NO:14, or a functional fragment or mutant thereof having at least 60%, at least 70%, at least 80%, at least 90%, at least 95%, at least 96%, at least 97%, at least 98% or at least 99% amino acid sequence identity to the amino acid sequence of SEQ ID NO:14;
   λ_{N22} comprising the amino acid sequence of SEQ ID NO:16, or a functional fragment or mutant thereof having at least 60%, at least 70%, at least 80%, at least 90%, at least 95%, at least 96%, at least 97%, at least 98% or at least 99% amino acid sequence identity to the amino acid sequence of SEQ ID NO:16; and
   PCP comprising the amino acid sequence of SEQ ID NO:306, or a functional fragment or mutant thereof having at least 60% at least 70%, at least 80%, at least 90%, at least 95%, at least 96%, at least 97%, at least 98% or at least 99% amino acid sequence identity to the amino acid sequence of SEQ ID NO:306.
E14: The AFP of any one of E1-E13 comprising at least one O-RS segment selected from:
   Methanococcus jannaschii tyrosyl-tRNA synthetase;
   Escherichia coli tyrosyl-tRNA synthetase;
   Escherichia coli leucyl-tRNA synthetase;
   Methanosarcina mazei pyrrolysyl-tRNA synthetase;
   Methanosarcina barkeri pyrrolysyl-tRNA synthetase;
   Methanosarcina acetivorans pyrrolysyl-tRNA synthetase;
   Methanosarcina thermophila pyrrolysyl-tRNA synthetase;
   Methanococcoides burtonii pyrrolysyl-tRNA synthetase;
   Desulfitobacterium hafniense pyrrolysyl-tRNA synthetase; and
   and functional fragments and mutants thereof which retain aminoacyl-tRNA synthetase enzymatic activity.
E15: The AFP of any one of E1-E14 comprising at least one O-RS segment selected from:
   PylRS^{AF} comprising the amino acid sequence of SEQ ID NO:8, or a functional fragment or mutant thereof having at least 60%, at least 70%, at least 80%, at least 90%, at least 95%, at least 96%, at least 97%, at least 98% or at least 99% amino acid sequence identity to the amino acid sequence of SEQ ID NO:8;
   PylRS^{AA} comprising the amino acid sequence of SEQ ID NO:10, or a functional fragment or mutant thereof having at least 60%, at least 70%, at least 80%, at least 90%, at least 95%, at least 96%, at least 97%, at least 98% or at least 99% amino acid sequence identity to the amino acid sequence of SEQ ID NO:10;
   PylRS^{AAAF} comprising the amino acid sequence of SEQ ID NO:12, or a functional fragment or mutant thereof having at least 60%, at least 70%, at least 80%, at least 90%, at least 95%, at least 96%, at least 97%, at least 98% or at least 99% amino acid sequence identity to the amino acid sequence of SEQ ID NO:12;
   IFRS1 comprising the amino acid sequence of SEQ ID NO:224, or a functional fragment or mutant thereof having at least 60%, at least 70%, at least 80%, at least 90%, at least 95%, at least 96%, at least 97%, at least 98% or at least 99% amino acid sequence identity to the amino acid sequence of SEQ ID NO:224;
   CbzRS comprising the amino acid sequence of SEQ ID NO:226; or a functional fragment or mutant thereof having at least 60%, at least 70%, at least 80%, at least 90%, at least 95%, at least 96%, at least 97%, at least 98% or at least 99% amino acid sequence identity to the amino acid sequence of SEQ ID NO:226;
   CpkRS comprising the amino acid sequence of SEQ ID NO:228 or a functional fragment or mutant thereof having at least 60%, at least 70%, at least 80%, at least 90%, at least 95%, at least 96%, at least 97%, at least 98% or at least 99% amino acid sequence identity to the amino acid sequence of SEQ ID NO:228; and
   OMeRS, comprising the amino acid sequence of SEQ ID NO:236 or a functional fragment or mutant thereof having at least 60%, at least 70%, at least 80%, at least 90%, at least 95%, at least 96%, at least 97%, at least 98% or at least 99% amino acid sequence identity to the amino acid sequence of SEQ ID NO:236.
E16: An assembler fusion protein (AFP) combination comprising at least two AFPs of any one of E1-E15.
E17: The AFP combination of E16 comprising at least one first AFP comprising at least one RNA-TP segment, and at least one second AFP comprising at least one O-RS segment.
E18: A fusion protein (RNA-TP/O-RS fusion protein) comprising:
   (i) at least one RNA-targeting polypeptide (RNA-TP) segment; and
   (ii) at least one orthogonal aminoacyl tRNA synthetase (O-RS) segment, wherein said polypeptide segments are functionally linked in said RNA-TP/O-RS fusion protein.
E19: The RNA-TP/O-RS fusion protein of E18 having one of the following structures (from the N-terminus to the C-terminus):
   [RNA-TP]ₓ-[O-RS]_{y}
   [O-RS]_{y}-[RNA-TP]ₓ
   wherein x and y, independently of each other, are integers selected from 1, 2, 3, 4 and 5; and “-” designates a peptidic linkage.
E20: The RNA-TP/O-RS fusion protein of E18 or E19 comprising at least one RNA-TP segment selected from RNA-binding segments of viral coat proteins, and functional fragments and mutants of RNA-binding segments of viral coat proteins which retain the ability to interact specifically with an RNA motif of the virus.
E21: The RNA-TP/O-RS fusion protein of any one of E18-E20 comprising at least one RNA-TP segment selected from:
   MCP comprising the amino acid sequence of SEQ ID NO:14, or a functional fragment or mutant thereof having at least 60%, at least 70%, at least 80%, at least 90%, at least 95%, at least 96%, at least 97%, at least 98% or at least 99% amino acid sequence identity to the amino acid sequence of SEQ ID NO:14;
   λ_{N22} comprising the amino acid sequence of SEQ ID NO:16, or a functional fragment or mutant thereof having at least 60%, at least 70%, at least 80%, at least 90%, at least 95%, at least 96%, at least 97%, at least 98% or at least 99% amino acid sequence identity to the amino acid sequence of SEQ ID NO:16; and
   PCP comprising the amino acid sequence of SEQ ID NO:306, or a functional fragment or mutant thereof having at least 60% at least 70%, at least 80%, at least 90%, at least 95%, at least 96%, at least 97%, at least 98% or at least 99% amino acid sequence identity to the amino acid sequence of SEQ ID NO:306.
E22: The RNA-TP/O-RS fusion protein of any one of E18-E21 comprising at least one O-RS segment selected from:
   Methanococcus jannaschii tyrosyl-tRNA synthetase;
   Escherichia coli tyrosyl-tRNA synthetase;
   Escherichia coli leucyl-tRNA synthetase;
   Methanosarcina mazei pyrrolysyl-tRNA synthetase;
   Methanosarcina barkeri pyrrolysyl-tRNA synthetase;
   Methanosarcina acetivorans pyrrolysyl-tRNA synthetase;
   Methanosarcina thermophila pyrrolysyl-tRNA synthetase;
   Methanococcoides burtonii pyrrolysyl-tRNA synthetase;
   Desulfitobacterium hafniense pyrrolysyl-tRNA synthetase; and
   and functional fragments and mutants thereof which retain aminoacyl-tRNA synthetase enzymatic activity.
E23: The RNA-TP/O-RS fusion protein of any one of E18-E22 comprising at least one O-RS segment selected from:
   PylRS^{AF} comprising the amino acid sequence of SEQ ID NO:8, or a functional fragment or mutant thereof having at least 60% sequence identity to the amino acid sequence of SEQ ID NO:8;
   PylRS^{AA} comprising the amino acid sequence of SEQ ID NO:10, or a functional fragment or mutant thereof having at least 60% sequence identity to the amino acid sequence of SEQ ID NO:10;
   PylRS^{AAAF} comprising the amino acid sequence of SEQ ID NO:12, or a functional fragment or mutant thereof having at least 60% sequence identity to the amino acid sequence of SEQ ID NO:12;
   IFRS1 comprising the amino acid sequence of SEQ ID NO:224, or a functional fragment or mutant thereof having at least 60%, at least 70%, at least 80%, at least 90%, at least 95%, at least 96%, at least 97%, at least 98% or at least 99% amino acid sequence identity to the amino acid sequence of SEQ ID NO:224;
   CbzRS comprising the amino acid sequence of SEQ ID NO:226; or a functional fragment or mutant thereof having at least 60%, at least 70%, at least 80%, at least 90%, at least 95%, at least 96%, at least 97%, at least 98% or at least 99% amino acid sequence identity to the amino acid sequence of SEQ ID NO:226;
   CpkRS comprising the amino acid sequence of SEQ ID NO:228 or a functional fragment or mutant thereof having at least 60%, at least 70%, at least 80%, at least 90%, at least 95%, at least 96%, at least 97%, at least 98% or at least 99% amino acid sequence identity to the amino acid sequence of SEQ ID NO:228; and
   OMeRS, comprising the amino acid sequence of SEQ ID NO:236 or a functional fragment or mutant thereof having at least 60%, at least 70%, at least 80%, at least 90%, at least 95%, at least 96%, at least 97%, at least 98% or at least 99% amino acid sequence identity to the amino acid sequence of SEQ ID NO:236.
E24: A nucleic acid molecule, or a combination of two or more nucleic acid molecules, comprising:
   (i) a nucleotide sequence that encodes at least one AFP of any one of E1-E15, or at least one AFP combination of E16 or E17, or
   (ii) a nucleic acid sequence complementary to the nucleotide sequence of (i).
   (iii) both of (i) and (ii).
E25: A nucleic acid molecule, or a combination of two or more nucleic acid molecules, comprising:
   (i) a nucleotide sequence that encodes at least one RNA-TP/O-RS fusion protein of any one of E18-E23, or
   (ii) a nucleic acid sequence complementary to (i), or
   (iii) both of (i) and (ii).
E26: An expression cassette comprising the nucleotide sequence of the nucleic acid molecule, or the combination of nucleic acid molecules, of E24 or E25.
E27: An expression vector comprising at least one expression cassette of E26.
E28: A cell comprising at least one nucleic acid molecule, or combination of nucleic acid molecules, of E24 or E25, at least one expression cassette of E26, or at least one expression vector of E27.
E29: The cell of E28 which is a eukaryotic cell.
E30: The cell of E28 which is a mammalian cell.
E31: The cell of any one of E28-E30 comprising at least one nucleic acid molecule, or combination of nucleic acid molecules, of E24, or at least one expression cassette comprising the nucleotide sequence of said nucleic acid molecule, or combination of nucleic acid molecules, or at least one expression vector comprising said expression cassette.
E32: The cell of E31 comprising a nucleotide sequence that encodes, or is complementary to a nucleotide sequence encoding, at least one AFP of any one of E1-E3 and E7-E15 comprising at least one EP selected from RNA-TP segments and at least one EP selected from O-RS segments.
E33: The cell of E31 comprising a nucleotide sequence that encodes, or is complementary to a nucleotide sequence encoding, at least one AFP of any one of E1-E3 and E7-E15 comprising at least one EP selected from RNA-TP segments, and at least one AFP of any one of E1-E3 and E7-E15 comprising at least one EP selected O-RS segments.
E34: The cell of any one of E28-E30 comprising at least one nucleic acid molecule, or combination of nucleic acid molecules, of E25, or at least one expression cassette comprising the nucleotide sequence of said nucleic acid molecule, or combination of nucleic acid molecules, or at least one expression vector comprising said expression cassette.
E35: The cell of any one E28-E34, wherein the cell expresses the at least one AFP, the at least one AFP combination or the at least one RNA-TP/O-RS fusion protein, respectively, that is encoded by the nucleotide sequence of said nucleic acid molecule, or combination of nucleic acid molecules.
E36: A method for preparing a polypeptide of interest (POI) comprising in its amino acid sequence one or more non-canonical amino acid (ncAA) residues, wherein the method comprises expressing the POI in a cell of any one of E31-E33 in the presence of said one or more ncAAs, wherein the cell comprises:
   (i) a POI-encoding nucleotide sequence (CS^{POI}) wherein said one or more ncAA residues of the POI are encoded by selector codon(s),
   (ii) a targeting nucleotide sequence (TN) that is functionally linked to the CS^{POI} and is able to interact with an RNA-TP segment of at least one of the AFPs in the cell;
   (iii) one or more orthogonal tRNA^{ncAA} (O-tRNA^{ncAA}) molecules which carry the anticodon(s) complementary to the selector codon(s) of the CS^{POI}, and wherein said O-tRNA^{ncAA} molecules together with one or more O-RS segments of at least one of the AFPs in the cell form one or more orthogonal O-RS/O-tRNA^{ncAA} pairs which allow for the introduction of said one or more ncAA residues into the amino acid sequence of the POI;
   and wherein the method optionally further comprises recovering the expressed POI.
E37: A method for preparing a polypeptide of interest (POI) comprising in its amino acid sequence one or more non-canonical amino acid (ncAA) residues, wherein the method comprises expressing the POI in a cell of E35 in the presence of said one or more ncAAs, wherein the cell comprises:
   (i) a POI-encoding nucleotide sequence (CS^{POI}) wherein said one or more ncAA residues of the POI are encoded by selector codon(s),
   (ii) a targeting nucleotide sequence (TN) that is functionally linked to the CS^{POI} and is able to interact with an RNA-TP segment of at least one of the RNA-TP/O-RS fusion proteins in the cell;
   (iii) one or more orthogonal tRNA^{ncAA} (O-tRNA^{ncAA}) molecules which carry the anticodon(s) complementary to the selector codon(s) of the CS^{POI}, and wherein said O-tRNA^{ncAA} molecules together with one or more O-RS segments of the RNA-TP/O-RS fusion proteins in the cell form one or more orthogonal O-RS/O-tRNA^{ncAA} pairs which allow for the introduction of said one or more ncAA residues into the amino acid sequence of the POI;
   and wherein the method optionally further comprises recovering the expressed POI.
E38: A method for preparing a polypeptide of interest (POI) comprising in its amino acid sequence one or more non-canonical amino acid (ncAA) residues, said method comprising the steps of:
   (a) expressing in a cell one or more AFPs of any one of E1-E3 and E7-E15 comprising at least one RNA-TP segment and one or more AFPs of any one of E1-E3 and E7-E15 comprising at least one O-RS segment;
   (b) expressing in said cell one or more orthogonal tRNA^{ncAA} (O-tRNA^{ncAA}) molecules, wherein
      said orthogonal tRNA^{ncAA} molecules and one or more of the O-RS segments of the AFPs in the cell form one or more orthogonal aminoacyl tRNA synthetase/tRNA^{ncAA} (O-RS/O-tRNA^{ncAA}) pairs,
      said O-RS/O-tRNA^{ncAA} pairs allow for introducing said one or more ncAA residues into the amino acid sequence of said POI,
   wherein steps (a) and (b) can be concomitantly or sequentially in any order;
   (c) then, expressing said POI in said cell in the presence of said one or more ncAAs, wherein
      the POI-encoding nucleotide sequence (CS^{POI}) comprises one or more selector codons encoding said one or more ncAA residues,
      said selector codons match the anticodons of said one or more O-tRNA^{ncAA} molecules;
      said CS^{POI} is functionally linked to a targeting nucleotide sequence (TN), thus forming a CS^{POI}/TN fusion sequence,
      said CS^{POI}/TN fusion sequence is able to interact, via its TN, with an RNA-TP segment of at least one of the AFPs in the cell;
   and
   (d) optionally recovering the expressed POI.
E39: A method for preparing a polypeptide of interest (POI) comprising in its amino acid sequence one or more non-canonical amino acid (ncAA) residues, said method comprising the steps of:
   (a) expressing in a cell RNA-TP/O-RS fusion proteins of any one of E18-E23; (b) expressing in said cell one or more orthogonal tRNA^{ncAA} (O-tRNA^{ncAA}) molecules, wherein
      said orthogonal tRNA^{ncAA} molecules and one or more of the O-RS segments of the RNA-TP/O-RS fusion proteins in the cell form one or more orthogonal aminoacyl tRNA synthetase/tRNA^{ncAA} (O-RS/O-tRNA^{ncAA}) pairs,
      said O-RS/O-tRNA^{ncAA} pairs allow for introducing said one or more ncAA residues into the amino acid sequence of said POI,
   wherein steps (a) and (b) can be concomitantly or sequentially in any order;
   (c) then, expressing said POI in said cell in the presence of said one or more ncAAs, wherein
      the POI-encoding nucleotide sequence (CS^{POI}) comprises one or more selector codons encoding said one or more ncAA residues,
      said selector codons match the anticodons of said one or more O-tRNA^{ncAA} molecules;
      said CS^{POI} is functionally linked to a targeting nucleotide sequence (TN), thus forming a CS^{POI}/TN fusion sequence,
      said CS^{POI}/TN fusion sequence is able to interact, via its TN, with an RNA-TP segment of at least one of the RNA-TP/O-RS fusion proteins in the cell;
   and
   (d) optionally recovering the expressed POI.
E40: The method of any one of E36-E39, wherein the TN is selected from viral RNA motifs bound by a viral coat protein, and functional fragments and mutants thereof which retain the ability to be bound by a viral coat protein.
E41: The method of any one of E36-E40, wherein the TN is selected from:
   MS2 RNA stem-loop comprising the RNA sequence encoded by the nucleotide sequence of SEQ ID NO:17, or a functional fragment or mutant thereof having at least 60%, at least 70%, at least 80%, at least 90%, at least 95%, at least 96%, at least 97%, at least 98% or at least 99% amino acid sequence identity to the amino acid sequence of SEQ ID NO:17;
   BoxB comprising the RNA sequence encoded by the nucleotide sequence of SEQ ID NO:18, or a functional fragment or mutant thereof having at least 60%, at least 70%, at least 80%, at least 90%, at least 95%, at least 96%, at least 97%, at least 98% or at least 99% amino acid sequence identity to the amino acid sequence of SEQ ID NO:18, and
   pp7 RNA stem-loop existing in at least two different versions and comprising the RNA sequence encoded by the nucleotide sequence of in particular a polynucleotide having an RNA sequence corresponding to (encoded by) the nucleotide (DNA) sequence of SEQ ID NO:289 or SEQ ID NO:290 or a functional fragment or mutant thereof having at least 60%, at least 70%, at least 80%, at least 90%, at least 95%, at least 96%, at least 97%, at least 98% or at least 99% amino acid sequence identity to the amino acid sequence of SEQ ID NO:289 or 290.
E42: The method of any one of E36-E41, wherein the selector codon(s) encoding the ncAA residue(s) of the POI are selected from Amber, Ochre and Opal stop codons.
E43: A nucleic acid molecule comprising:
   (i) a nucleotide sequence (CS^{POI}) that encodes a polypeptide of interest (POI), said POI comprising one or more non-canonical amino acid (ncAA) residues which are encoded in the CS^{POI} by selector codons, and
   (ii) a targeting nucleotide sequence (TN), wherein an RNA molecule comprising said TN is able to interact via said TN with an RNA-targeting polypeptide (RNA-TP).
E44: The nucleic acid molecule of E43, wherein the TN is selected from viral RNA motifs bound by a viral coat protein, and functional fragments and mutants thereof which retain the ability to be bound by a viral coat protein.
E45: The nucleic acid molecule of E43 or E44, wherein the TN is selected from:
   MS2 RNA stem-loop comprising the RNA sequence encoded by the nucleotide sequence of SEQ ID NO:17, or a functional fragment or mutant thereof having at least 60%,%, at least 70%, at least 80%, at least 90%, at least 95%, at least 96%, at least 97%, at least 98% or at least 99% amino acid sequence identity to the amino acid sequence of SEQ ID NO:17;
   BoxB comprising the RNA sequence encoded by the nucleotide sequence of SEQ ID NO:18, or a functional fragment or mutant thereof having at least 60%, %, at least 70%, at least 80%, at least 90%, at least 95%, at least 96%, at least 97%, at least 98% or at least 99% amino acid sequence identity to the amino acid sequence of SEQ ID NO:18; and
   pp7 RNA stem-loop existing in at least two different versions and comprising the RNA sequence encoded by the nucleotide sequence of in particular a polynucleotide having an RNA sequence corresponding to (encoded by) the nucleotide (DNA) sequence of SEQ ID NO:289 or SEQ ID NO:290 or a functional fragment or mutant thereof having at least 60%, at least 70%, at least 80%, at least 90%, at least 95%, at least 96%, at least 97%, at least 98% or at least 99% amino acid sequence identity to the amino acid sequence of SEQ ID NO:289 or 290.
E46: The nucleic acid molecule of any one of E43-E45, wherein the selector codon(s) encoding the ncAA residue(s) of the POI are selected from Amber, Ochre and Opal stop codons.
E47: A kit for preparing a polypeptide of interest (POI) having at least one non-canonical amino acid (ncAA) residue, the kit comprising:
   at least one ncAA, or salt thereof, corresponding to the at least one ncAA residue of the POI, and
   at least one expression vector of E27.
E48: The kit of E47, wherein the expression vector encodes a fusion protein comprising at least one O-RS segment and at least one RNA-TP segment.
E49: The kit of E47 or E48 further comprising at least one expression vector for an orthogonal tRNA^{ncAA} (O-tRNA^{ncAA}) molecule.
E50: The kit of any one of E47-E49 further comprising at least one expression vector comprising a multiple cloning site and a targeting nucleotide sequence (TN), wherein an RNA molecule comprising said TN is able to interact via said TN with an RNA-targeting polypeptide (RNA-TP).
   Anyone of the above embodiments also encompass the following modification: The above-mentioned AP (i.e. IC-TP and PSP) segments and/or EP (RNA-TP or O-RS) segments may be further combined with synthetic protein segments, which induce and control macromolecular interactions. One or more, like 2, 3, 4, 5, 6, 7, 8, 9, or 10, preferably one such protein segment may be operably fused into a single AFP of the invention. Of particular interest in the context of the invention are SYNZIPs having the ability to form heterodimeric coiled-coil protein structures. Such SYNZIPs are pairs of synthetic peptides capable of interacting with each other and are used to induce and control macromolecular interactions. Non-limiting examples are the pairs SYNZIP 1:2; SYNZIP 3:4 and SYNZIP 5:6. Particularly preferred according to the invention is the heterospecific coiled-coil pair SYNZIP2:SYNZIP1 as described by Reinke, A. W., Grant, R. A., Keating, A. E. (2010) J Am Chem Soc 132 6025-6031 (SYNZIP 1: SEQ ID NO:312, SYNZIP 2: SEQ ID NO:314, SYNZIP 3: SEQ ID NO:316; SYNZIP 4: SEQ ID NO:318, as well as functional fragments and mutants of these SYNZIP polypeptides. Said functional fragments and mutants may comprise at least 60%, at least 70%, at least 80%, at least 90%, at least 95%, at least 96%, at least 97%, at least 98% or at least 99% amino acid sequence identity to the amino acid of the polypeptide they are derived from.

The invention is further illustrated by the following non-limiting examples.

### Examples

Methods

(A) Cell Culture, Transfections and Feeding with ncAAs

HEK293T cells (ATCC CRL-3216) and COS-7 cells (ATCC, CRL-1651) were maintained in Dulbecco's modified Eagle's medium (Life Technologies, 41965-039) supplemented with 1% penicillin-streptomycin (Sigma, 10,000 U/ml penicillin, 10 mg/ml streptomycin, 0.9% NaCl), 2 mM L-glutamine (Sigma), 1 mM sodium pyruvate (Life Technologies) and 10% FBS (Sigma). Cells were cultured at 37° C. in a 5% CO₂ atmosphere and passaged every 2-3 days up to 15-20 passages.

In all cases, cells were seeded 15-20 h prior to transfection at a density resulting in 70-80% confluency at the time of transfection. Flow cytometry was performed using 24-well plates with plastic bottom (Nunclon Delta Surface ThermoScientific). Immunofluorescence labeling and FISH were performed on 24-well plates with glass bottom (Greiner Bio-One) or four-well Lab-Tek #1.0 borosilicate coverglass (ThermoFisher).

Transfections of HEK293T cells were performed with polyethylenimine (PEI, Sigma-Aldrich) using 3 μg PEI per 1 μg DNA. COS-7 cells were transfected using the JetPrime reagent (PeqLab) according to the manufacturer's recommendations at a ratio of 1:2.

For Amber suppression system test, cells were transfected at a ratio of a 1:1:1:1 with POI^{TAG} vectors, tRNA^{Pyl}, synthetase and MCP or mock constructs. 4-6 hours after transfection the medium to a fresh containing ncAA.

Stock and working solutions for all of the used ncAAs were prepared as described in Nikic et al. (Nat Protoc 10(5):780-791, 2015). SCO (cyclooctyne lysine, SiChem SC-8000) was used at a final concentration of 250 μM. 3-Iodophenylalanine (Chem-Impex International Inc.) was used at a final concentration of 1 mM. SCO is efficiently recognized by PylRS^{AF} (Y306A, Y384F) (see Plass et al., Angew Chem 2011, 50:3878-3881). 3-Iodophenylalanine is recognized by PylRS^{AA} (C346A, N348A) (see Wang et al., ACS Chem Biol 2013, 8:405-415).

(B) Flow Cytometry

HEK293T cells were harvested after one day after transfection, resuspended in 1×PBS and passed through 100 μm nylon mesh. Co-transfections for flow cytometry were performed at a 1:1:1:1 ratio with 1.2 μg total DNA with:
a reporter plasmid encoding the POI (a stop codon encoding the amino acid position to be occupied by the ncAA),
a plasmid encoding the tRNA^{Pyl} having the anticodon which matches (i.e., is the reverse complement) of the stop codon in the POI-encoding sequence (hereinafter simply referred to as tRNA^{Pyl}),
a plasmid encoding the PylRS or functional mutant thereof, respectively, and
either a plasmid encoding an MCP fusion polypeptide or a mock plasmid.

Cell culture medium was exchanged for fresh medium containing the ncAA to be incorporated into the POI 4-6 h post-transfection and left until the time of harvesting.

Data acquisition and analysis were performed using an LSRFortessa SORP Cell Analyzer (Becton, Dickinson and Company) and the FlowJo software (FlowJo). Cells were first gated by cell type using forward scatter area (FSC-A) and side scatter area (SSC-A) parameters. Subsequently, single cells were identified based on SSC-A and side scatter width (SSC-W). Each shown FFC plot is the sum of three independent biological replicates from which the mean and the SEM were calculated. At least 130,000 single cells were analyzed per condition. GFP fluorescence was acquired in the 488-530/30 channel and mCherry fluorescence in the 561-610/20 channel.

(C) PylRS immunostaining and imaging, fluorescence in situ hybridization (FISH)

For immune-labelling experiments, the cells were rinsed with 1×PBS, fixed in 2% paraformaldehyde in 1×PBS for 10 min at RT, rinsed with 1×PBS again and then permeabilized in 0.5% Triton X in 1×PBS for 15 min at RT. After rinsing the permeabilized cell samples twice with 1×PBS, said samples were incubated for 90 min in blocking solution (3% BSA in 1×PBS for 90 min at RT), and then with 1 μg/ml primary antibody (polyclonal rat anti-PylRS, prepared as described in Nikic et al. (Angew Chem Int Ed Engl 2016, 55(52):16172-16176) and/or polyclonal rabbit anti-MCP (Merck, ABE76) and/or monoclonal rabbit anti-RPL26L1 antibody (EPR8478, Abcam, ab137046)) in blocking solution overnight at 4° C. The next day, the cell samples were rinsed with 1×PBS and incubated with 2 μg/ml secondary antibody (chicken anti-rat IgG(H+L) cross-adsorbed Alexa Fluor 594 conjugated antibody (Thermo Fisher Scientific, A-21471) and/or goat anti-rabbit IgG(H+L) cross-adsorbed Alexa Fluor 647 conjugated F(ab′)₂ (Thermo Fisher Scientific, A-21246)) in blocking solution for 60 min at RT. DNA was stained with Hoechst 33342 (1 μg/ml in 1×PBS) for 10 min at RT. If only DNA was stained, the cells were fixed and permeabilized as described above and then stained with Hoechst 33342 (1 μg/ml in 1×PBS) for 10 min at RT. Finally, the cells were rinsed twice with 1×PBS.

FISH experiments were performed one day after transfection analogously to the FISH experiments described in Nikic et al. (Angew Chem Int Ed Engl 2016, 55(52):16172-16176). The hybridization protocol was adapted for 24-well plates from Pierce et al. (Methods Cell Biol 122:415-436, 2014).

For imaging of only tRNA^{Pyl}, the hybridization probe 5′-CTAACCCGGCTGAACGGATTTAGAGTCCATTCGATC-3′ (labelled at the 5′ terminus with Cy5; SEQ ID NO:1) was used at 0.25 μM. After four washes with SSC and one wash with TN buffer (0.1 M TrisHCl, 150 mM NaCl), cells were incubated for 1 h at RT with 3% BSA in TN buffer prior to standard immunofluorescence labeling as described above.

For imaging of both tRNA^{Pyl} and MS2 RNA stem-loop sequence, the hybridization probe for tRNA^{Pyl} (5′-CTAACCCGGCTGAACGGATTTAGAGTCCATTCGATC-3′, labelled at the 5′ terminus with digoxigenin; SEQ ID NO:2) was used at 0.16 μM, and the hybridization probe for the MS2 RNA stem-loop sequence (5′-CTGCAGACATGGGTGATCCTCATGTTTTCTA-3′, labelled at the 5′ terminus with Alexa Fluor 647; SEQ ID NO:3) was used at 0.75 μM. After four washes with SSC, the cells were incubated for 1 h at RT in blocking buffer (0.1 M TrisHCl, 150 mM NaCl, 1× blocking reagent (Sigma 11096176001). Then, the cells were incubated with fluorescein conjugated sheep anti-digoxigenin Fab (Sigma 11207741910) at a 1:200 dilution in blocking buffer overnight at 4° C. The next day, 3 washes of 5 minutes were done in Tween buffer (0.1 M TrisHCl, 150 mM NaCl, 0.5% Tween20). DNA was stained with Hoechst 33342 (1 μg/ml in 1×PBS) for 10 min at RT.

Confocal images were acquired on a Leica SP8 STED 3× microscope equipped with a 63×/1.40 oil immersion objective using the following laser lines for excitation: 405 nm for Hoechst 33342, 488 nm for fluorescein and GFP, 548 nm for mOrange, 594 nm for Alexa Fluor 594, 647 nm for Alexa Fluor 647 and Cy5. Emission light was collected with HyD detectors at 420-500 nm and 605-680 nm respectively.

Ribosomal immunofluorescence images were taken on an Olympus Fluoroview FV3000 microscope equipped with a 60×/1.40 oil immersion objective using the following laser lines for excitation: 488 nm for GFP, 594 nm for Alexa Fluor 594, 640 nm for Alexa Fluor 647.

Images were processed using FIJI software.

(D) Constructs, Cloning and Mutagenesis

Two different fluorescent protein reporters (dual color reporter) were cloned into a pBI-CMV1 vector (Clontech 631630), one protein in one multiple cloning site and the other reporter in the other multiple cloning site. The CDS for one of the reporters encoded an mRNA carrying two MS2 RNA stem-loops fused to the 3′ untranslated region (“MS2-tag”), while the encoded mRNA of the other reporter was not MS2-tagged.

For examination of Amber suppression, The reporters GFP^{39TAG} and mCherry^{185TAG} were used as N-terminal fusion with NLS. For examination of Ochre and Opal suppression, analogous constructs were prepared (with GFP^{39TAA} and mCherry^{185TAA}, GFP^{39TGA} and mCherry^{185TGA}, respectively).

NLS::GFP^{39TAG}::MS2-tag reporter: NLS::GFP^{39TAG} was cloned with two copies of MS2 RNA stem-loops into the pBI-CMV1 vector as a reporter for successful Amber suppression in imaging experiments.

For examination of suppression of multiple Amber codons, pBI-CMV constructs for GFP^{39,149TAG} and GFP^{39,149,182TAG} were prepared which did not contain a second (e.g. mCherry) reporter in the second multiple cloning site.

Further non-limiting examples of GFPs which are applicable in the context of the invention are:

GFP^{66TAG} GFP with Amber site (SEQ ID NO:238)

GFP^{66TCG} GFP with Serine site (SEQ ID NO:240)

GFP^{66CCG} GFP with Proline site (SEQ ID NO:242)

GFP^{66CTA} GFP with Leucine site (SEQ ID NO:244)

GFP^{66TTA} GFP with Leucine site (SEQ ID NO:246)

GFP^{66ATA} GFP with Isoleucine site (SEQ ID NO:248)

GFP^{66CGG} GFP with Arginine site (SEQ ID NO:250)

GFP^{39TCG} GFP with Serine site (SEQ ID NO:252)

GFP^{39CCG} GFP with Proline site (SEQ ID NO:254)

GFP^{39CTA} GFP with Leucine site (SEQ ID NO:256)

GFP^{39CGG} GFP with Arginine site (SEQ ID NO:258)

GFP^{39TCG} LCK-GFP with Serine site (SEQ ID NO:278)

GFP^{39CCG} LCK-GFP with Proline site (SEQ ID NO:280)

GFP^{39CTA} LCK-GFP with Leucine site (SEQ ID NO:282)

Extended GFP^{39TCG} GFP with Serine site at position 39 genetically fused to GFP^{66CCG} (SEQ ID NO:284)

Extended GFP^{39CCG} GFP with Proline site at position 39 genetically fused to GFP^{66TCG} (SEQ ID NO:286)

Extended GFP^{39CTA} GFP with Leucine site at position 39 genetically fused to GFP^{66TCG}(SEQ ID NO:288)

Further non-limiting examples of mCherrys which are applicable in the context of the invention are:

mCherry^{72TAG} mCherry with Amber site (SEQ ID NO:260)

mCherry^{72TCG} mCherry with Serine site (SEQ ID NO:262)

mCherry^{72CCG} mCherry with Proline site (SEQ ID NO:264)

mCherry^{72CTA} mCherry with Leucine site (SEQ ID NO:266)

mCherry^{72TTA} mCherry with Leucine site (SEQ ID NO:268)

mCherry^{72ATA} mCherry with Isoleucine site (SEQ ID NO:270)

mCherry^{185TCG} mCherry with Serine site (SEQ ID NO:272)

mCherry^{185CCG} mCherry with Proline site (SEQ ID NO:274)

mCherry^{185CTA} mCherry with Leucine site (SEQ ID NO:276)

Further non-limiting examples of mCherry constructs comprising different TN loops which are applicable in the context of the invention are:

mCherry^{190TAG}-2×PP7 mCherry with amber site and 2× pp7 loops (SEQ ID NO:216)

mCherry^{190TAG}-4×PP7 mCherry with amber site and 4× pp7 loops (SEQ ID NO:218)

mCherry^{190TAG}-6×PP7 mCherry with amber site and 6× pp7 loops (SEQ ID NO:220)

H2B-mCherry^{190TAG}-2×MS2 Human Histone H2B type 1-J (Uniprot: P06899) fused to mCherry with amber site and 2× ms2-loops (SEQ ID NO:222)

They may be fused into the polypeptide chain of any of the AFP molecules described herein, in particular at a position within the fusion molecule which does not inhibit the function of anyone of the other polypeptide segments (APs and EPs) of the AFP molecule. Examples of such epitope-tag containing AFP molecules are given below.

Constructs for OT assemblies were prepared as follows: tRNA^{Pyl} was cloned under the control of a human U6 promoter, and all other constructs were under CMV promoters cloned in the pcDNA3.1 (Invitrogen V86020) vector. MCP protein was cloned from the addgene plasmid #31230 and FUS from the Addgene plasmid #26374. In all FUS fusions, amino acids 1-478(S108N) were used, replacing the C-terminal NLS region by a Flag-tag. In all RS fusions the previously reported efficient NES::PylRS^{AF} (Y306A, Y384F) sequence was used (see, e.g., Nikic et al., Angew Chem Int Ed Engl 2016, 55(52):16172-16176). The PylRS mutant PylRS^{A} (N346A, C348A) was cloned via site-directed mutagenesis starting from wildtype PylRS. The SPD5 gene was ordered from Genewiz and fused to MCP and PylRS^{AF} via restriction cloning. KIF13A₁₋₄₁₁ and KIF16B₁₋₄₀₀ were cloned from human cDNA and inserted into pcDNA3.1 via restriction cloning. P390 of KIF13A₁₋₄₁₁ was removed via side directed mutagenesis. KIF13A₁₋₄₁₁,ΔP390 and KIF16B.₄₀₀ fusions with MCP, PylRS^{AF}, EWSR1::MCP, FUS::PylRS^{AF}, FUS::PylRS^{AA}, SPD5::MCP and SPD5::PylRS^{AF} were assembled via Gibson assembly (see Gibson et al., Nat Methods 2009, 6:343-345).

Constructs for differential imaging experiments: To selectively express Nup153-EGFP^{149TAG} and Vim^{116TAG}-mOrange, one gene was first inserted together with an MS2-tag into pBI-CMV1 (compare Nikic et al., Angew Chem Int Ed Engl 2016, 55(52):16172-16176). Subsequently, the other gene was inserted without MS2-tag. INSR^{676TAG}::mOrange was fused to an MS2-tag by replacing Vim^{116TAG}-mOrange in the pBI vector bearing Nup153::EGFP^{149TAG} and Vim^{116TAG}::mOrange::MS2-tag to yield a bicistronic vector with INSR^{676TAG}::mOrange in one and Nup153::EGFP^{149TAG} in the other cassette.

Multicistronic Amber suppression vectors for COS-7 cell experiments: As COS-7 cells have lower transfection efficiency; we generated multicistronic vectors harboring the components of an OT assembly. To assemble multicistronic Amber suppression vectors, first one copy of tRNA^{Pyl} under the control of a human U6 promoter was inserted into the pBI-CMV1 vector via Gibson assembly. Subsequently, first the AFP CDS KIF16B::FUS::PylRS^{AF} and finally the AFP CDS KIF16B::EWSR1::MCP were inserted via Gibson assembly. Alternatively, a previously published pcDNA3.1 based construct (see Nikic et al., Angew Chem Int Ed Engl 2016, 55(52):16172-16176) expressing NES::PylRS^{AF} under a CMV promoter and tRNA^{Pyl} under a human U6 promoter was used. Constructs with U6-tRNA^{Pyl}, KIF16B::FUS::PylRS^{AF} and KIF16B::EWSR1::MCP, or NES::PylRS^{AF} were alternatively inserted into a pDonor vector (GeneCopoeia).

The respective sequence information on AFPs used in the following experiments can be taken from the listing of sequences given below.

### Example 1—RNA-TP/O-RS Fusion and AFPs Comprising a Single AP

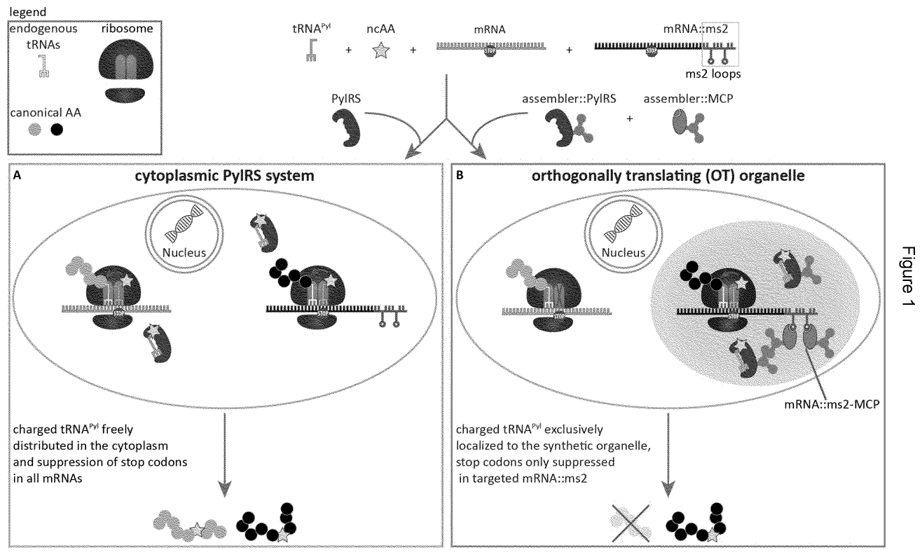

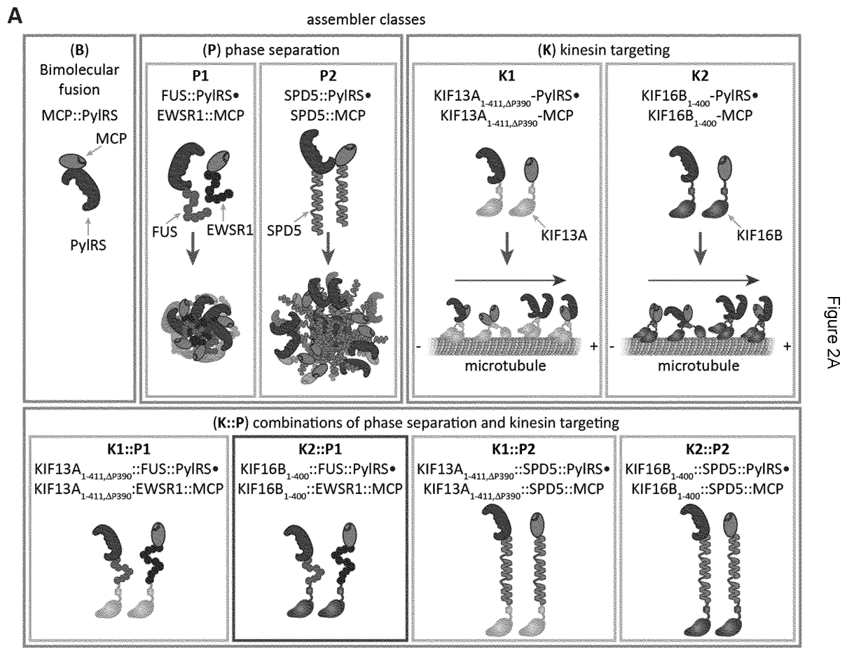

An OT assembly (“OT organelle”, FIG. 1) was engineered having the following components:

i) An mRNA-targeting system in which two MS2 RNA stem-loops (MS2-tag) were fused to the mRNA of choice coding for the POI, creating an mRNA::ms2 fusion. The MS2-tag binds specifically to the MS2 bacteriophage coat protein (MCP) (see Bertrand et al., Mol Cell 1998, 2:437-445), which will thus form a stable and specific mRNA::ms2-MCP complex in cells. The MS2-tag was always fused to the 3′ untranslated region (3′ UTR) of the mRNA, which ensures translation to yield a scar-less final POI.

ii) A tRNA/RS suppressor pair. The orthogonal tRNA/RS pair from the Methanosarcina mazei pyrrolysyl system (tRNA^{Pyl}/PylRS) was chosen because it has enabled the encoding of more than 200 ncAAs with diverse functionalities into proteins using GCE in a multitude of cell types and species, including E. coli, mammalian cells and even living mice (see, e.g., Liu et al., Annu Rev Biochem 2010, 79:413-444; Lemke, ChemBioChem 2014, 15:1691-1694; Chin, Nature 2017, 550; 53-60).

iii) The assembler (AP) was the key component required to form an OT assembly. The purpose of the assembler was to create membrane-less structures in the form of a dense phase, aggregate, droplet or condensate, in which the mRNA::ms2-MCP complex is brought into close proximity of the tRNA^{Pyl}/PylRS pair.

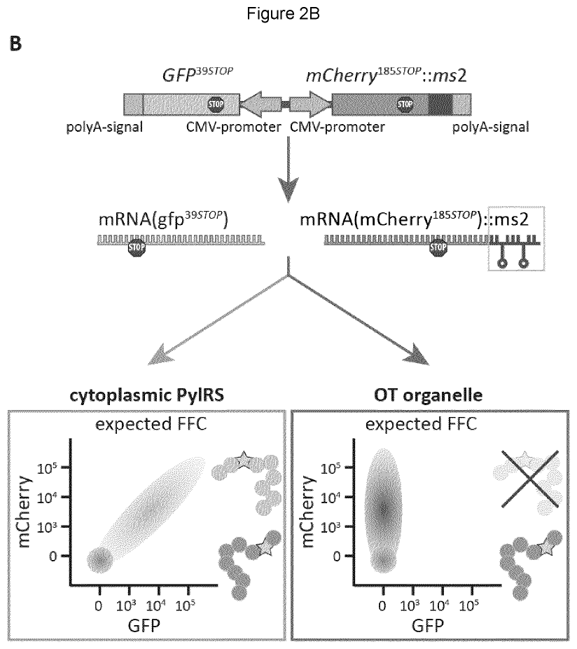

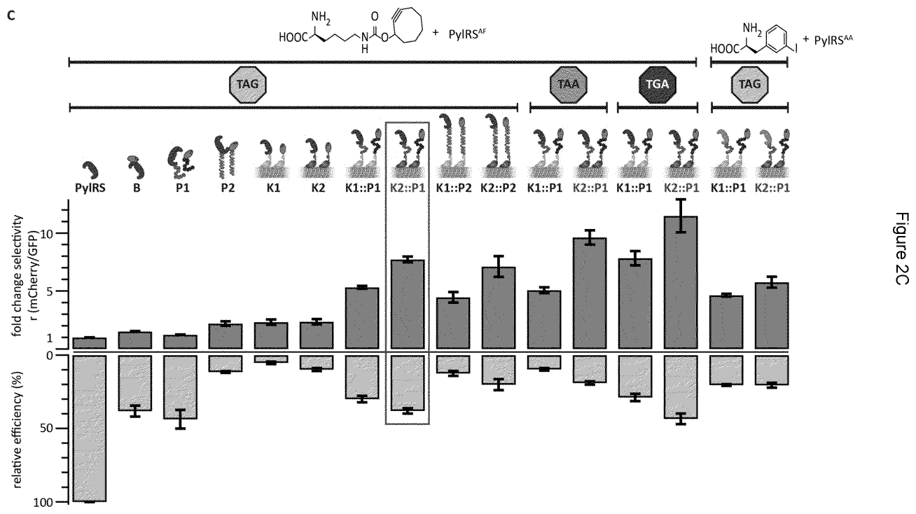

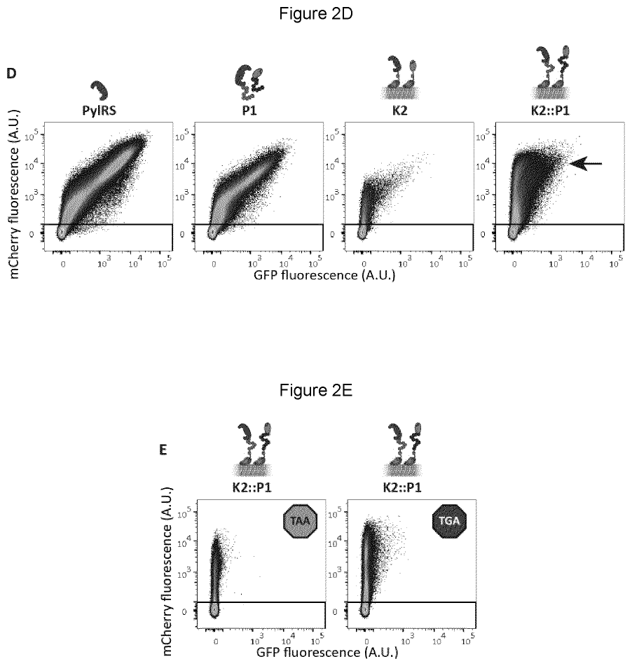

The simplest strategy tested was the bimolecular fusion of MCP::PylRS (termed B, FIG. 2). In addition, strategies were tested which were expected to yield much larger assemblies. All of those assembly systems were composed of an assembler fusion to PylRS co-expressed with an assembler fusion to MCP. Assembler::PylRS⋅assembler::MCP were expected to form large aggregates (co-expression herein denoted with “⋅”). One tested assembly strategy was based on phase separation of proteins and one based on the assembly of kinesins, which are abbreviated herein as P and K, respectively (FIG. 2A). Furthermore, for each P and K approach two different molecular designs (P1, P2 and K1, K2, respectively) were tested which are summarized as follows:

P1. Previous studies have established the capacity of the proteins fused-in sarcoma (FUS) and Ewing sarcoma breakpoint regions 1 (EWSR1) to form mixed droplet-like structures by phase separation. They both contain a prion-like disordered domain that facilitates phase separation into liquid, gel and solid states (see, e.g., Altmeyer et al., Nat Commun 2015, 6:8088; Patel et al., Cell 2015, 162:1066-1077). In a phase-separated state, these proteins are locally highly concentrated (several orders of magnitude) compared to the remaining soluble fraction in the cytoplasm. FUS was fused to PylRS and EWSR1 was fused to MCP. It was expected that this would lead to the formation of droplets in which MCP and PylRS are highly enriched. P1 is denoted FUS::PylRS⋅EWSR1::MCP.

P2. The Caenorhabditis elegans protein spindle-defective protein 5 (SPD5) has been shown to phase separate into particularly large (several micron-sized) droplets (see Woodruff et al., Cell 2017, 169:1066-1077, e1010). In a phase-separated state, SPD5 is locally highly concentrated compared to the remaining soluble fraction in the cytoplasm (by several orders of magnitude). It was expected that a protein fused to SPD5 would condense into droplets. Similarly to FUS-EWSR1 droplets, PylRS fused to SPD5 and MCP fused to SPD5 were expected to be highly enriched. P2 is denoted SPD5::PylRS⋅SPD5::MCP.

K1. Certain kinesin truncations constitutively move towards microtubule-plus ends in living cells (Soppina et al., Proc Natl Acad Sci U.S.A. 2014, 111:5562-5567). One such truncated kinesin is KIF13A_{1-411,ΔP390}, and it was expected that PylRS and MCP, respectively, fused to this kinesin truncation and co-expressed would be locally enriched, due to spatial targeting to microtubule-plus ends. K1 is denoted KIF13A_{1-411,ΔP390}::PylRS⋅KIF13A_{1-411,ΔP390}::MCP.

K2. By analogy to K1, the truncated kinesin KIF16B₁₋₄₀₀ was also tested. K2 is denoted KIF16B₁₋₄₀₀::PylRS⋅KIF16B₁₋₄₀₀::MCP.

In order to evaluate these assemblers for facilitating functional orthogonal translation of an MS2-tagged mRNA, a dual-reporter construct was designed, in which GFP and mCherry mutants are simultaneously expressed from two different expression cassettes from one plasmid, ensuring that the mRNA ratio between them is constant across all experiments. Stop codons were introduced at permissive sites into GFP at position 39 (GFP^{39STOP}) and into mCherry at position 185 (mCherry^{185STOP}; FIG. 2B). Only if stop codon suppression is successful will the corresponding green or red fluorescent protein be produced. Transfected cells (tRNA^{Pyl} and ncAA were always present unless specifically noted otherwise) were analyzed by fluorescence flow cytometry (FFC); settings were adjusted so that an approximate diagonal results in the FFC plots if GFP and mCherry are expressed from this plasmid using the conventional cytoplasmic PylRS system, which cannot differentiate mRNAs. A selective and functional OT organelle should selectively express mCherry only if the MS2-tag is fused to the 3′ UTR of the mCherry mRNA, leading to the appearance of a vertical line in the cytometry plot (FIG. 2B). Unless otherwise reported, all experiments where performed in the presence of tRNA^{Pyl} and the ncAA SCO, a widely used and well characterized lysine derivative, the side chain of which carries a cyclooctyne that can be used in a variety of click-chemistry reactions to install diverse chemical groups onto the protein. As previously reported, this ncAA is efficiently encoded by a Y306A, Y384F double mutant of PylRS (for simplicity this mutant is designated PylRS herein, unless otherwise specified) (see Nikic et al., Angew Chem 2014, 53:2245-2249; Plass, Angew Chem 2012, 51:4166-4170; Plass et al., Angew Chem 2011, 50:3878-3881). Omission of the ncAA served as a standard negative control and lead to no expression of GFP or mCherry.

The performance of each OT system was evaluated according to its selectivity and relative efficiency. Selectivity is defined as the ratio r of the mean mCherry FFC signal divided by the mean GFP signal. Final values are expressed as fold selectivity relative to that of cytoplasmic PylRS. Relative efficiency is defined as the mean mCherry signal of each system divided by the mean mCherry signal of the cytoplasmic PylRS system, which serves as the reference (here defined as 100%). All results on selectivity (dark-gray positive bars) and efficiency (light-gray negative bars) are summarized in the bar plot in FIG. 2C. Selected FFC data is also shown in FIG. 2D.

The simplest strategy B (MCP fused to PylRS) showed an about 1.5-fold selectivity gain (FIG. 2C). The OT system P1 (based on phase separation of FUS/EWSR1) had a somewhat lower selectivity gain (FIG. 2C, D). The P2 system (based on SPD5) showed an approximate twofold selectivity gain (FIG. 2C). For K1 a twofold increase in selectivity was observed (FIG. 2C). The K2 system behaved similarly (FIG. 2C,D). In total, the selectivity gains were relatively small, but robustly detected and distinguishable from a simple efficiency drop. The observed selectivity effect (data not shown) was robust across a titration of Amber suppression efficiencies (specifically, 0.48 ng, 2.4 ng, 12 ng, 60 ng or 300 ng tRNA^{Pyl} construct, respectively, were used), indicating that bringing the ncAA aminoacylation activity (i.e. the tRNAPyl/PylRS in the presence of ncAA) in direct proximity of the target mRNA represents a pathway to more selective codon suppression.

### Example 2—AFPs Comprising a Combination of Two APs

AFPs comprising combinations of the APs described in example 1 were tested in an analogous manner, those were:

K1::P1=KIF13A_{1-411,ΔP390}::FUS::PylRS⋅KIF13A_{1-411,ΔP390}::EWSR1::MCP,

K2::P1=KIF16B₁₋₄₀₀::FUS::PylRS⋅KIF16B₁₋₄₀₀::EWSR1::MCP,

K1::P2=KIF13A_{1-411,ΔP390}::SPD5::PylRS⋅KIF13A_{1-411,ΔP390}::SPD5::MCP,

K2::P2=KIF16B₁₋₄₀₀::SPD5::PylRS⋅KIF16B₁₋₄₀₀::SPD5::MCP.

For all combinations an at least fivefold selectivity gain was observed indicating orthogonal translation. The best performing of these systems was based on the fusion of FUS/EWSR1 with KIF16B₁₋₄₀₀, K2::P1 and exhibited a selectivity of eightfold (box in FIG. 2C). This was also directly obvious from the FFC data, in which the bright, mCherry-positive cell population was clearly retained, whereas GFP expression was minimal (arrow in FIG. 2D).

### Example 3—AFPs Comprising a Combination of APs Including a Membrane-Targeting AP

AFPs comprising combinations of APs derived from phase separation polypeptides (PSPs), FUS and EWSR1 (also termed EWS herein), optionally fused to SYNZIP segments, and different APs which acts as a membrane-targeting signal, LcK, EB1, CG1, EBAG9_{full length}, EBAG9₁₋₂₉, CMP Sia Tr, P450 2C1₁₋₂₇ and P450 2C1₁₋₂₉ were tested in a manner analogous to example 2.

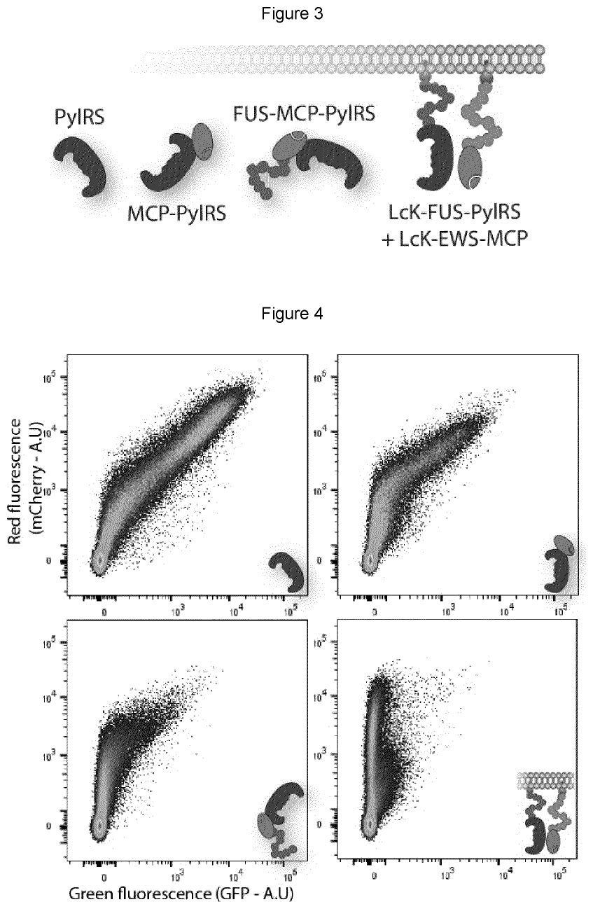

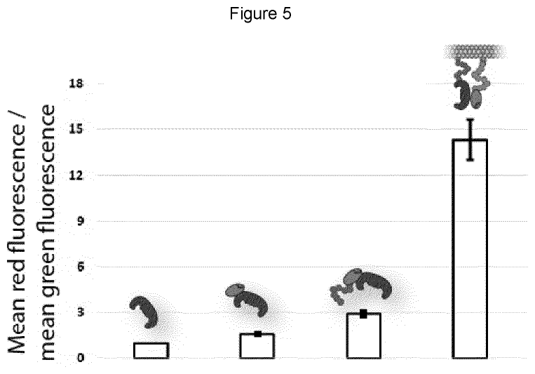

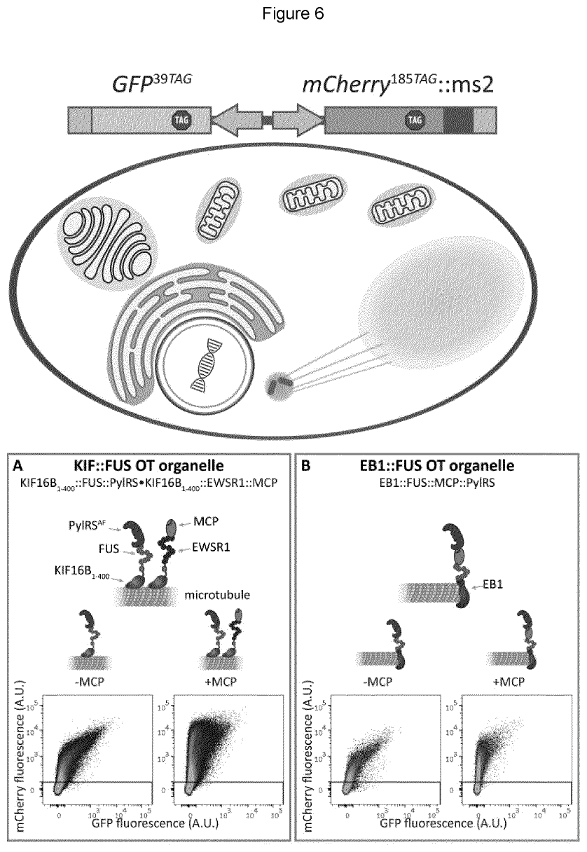

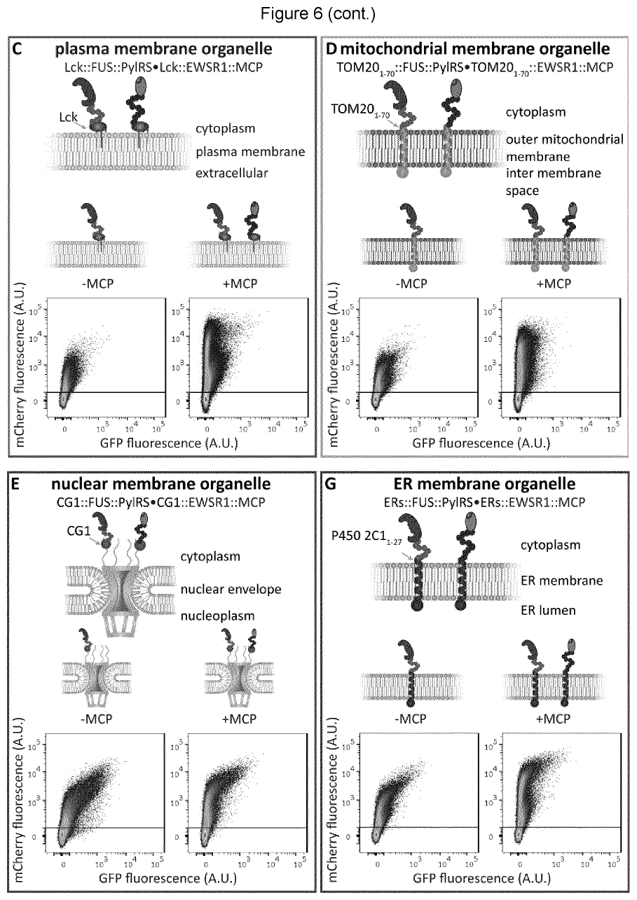

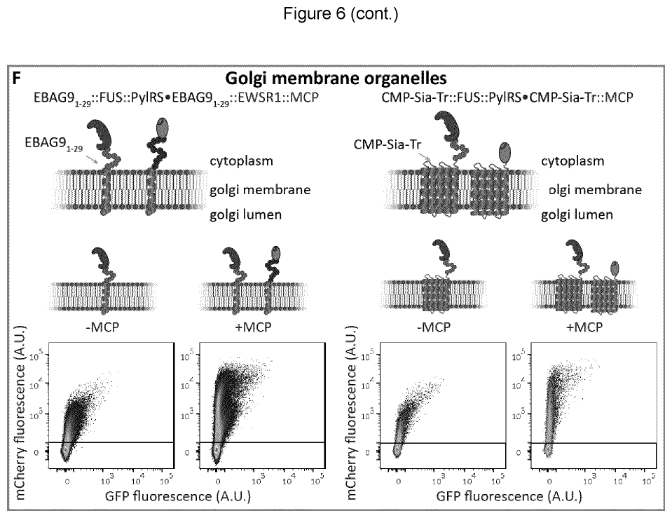

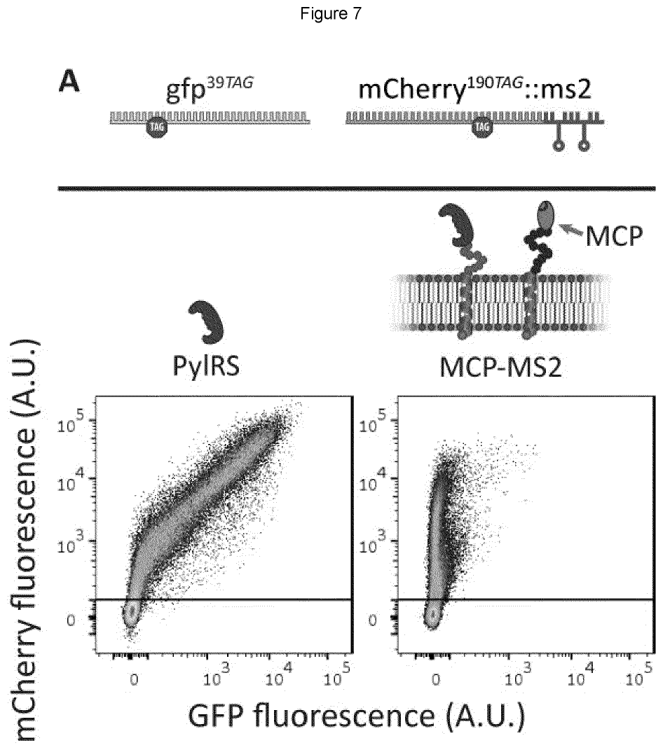

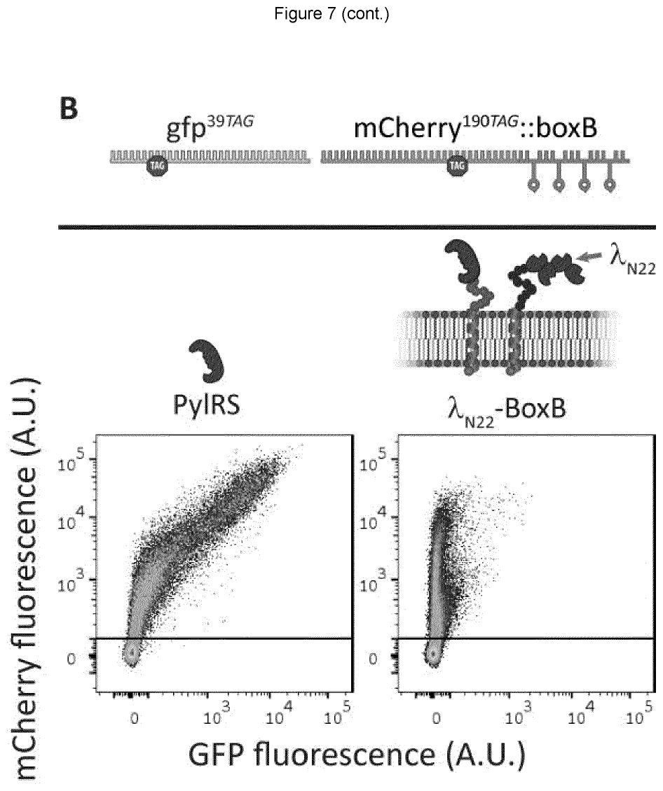

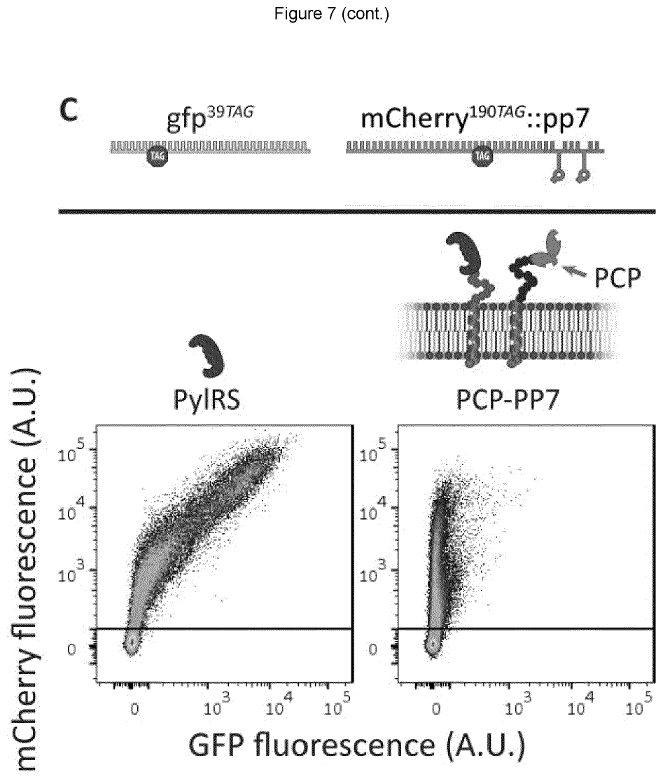

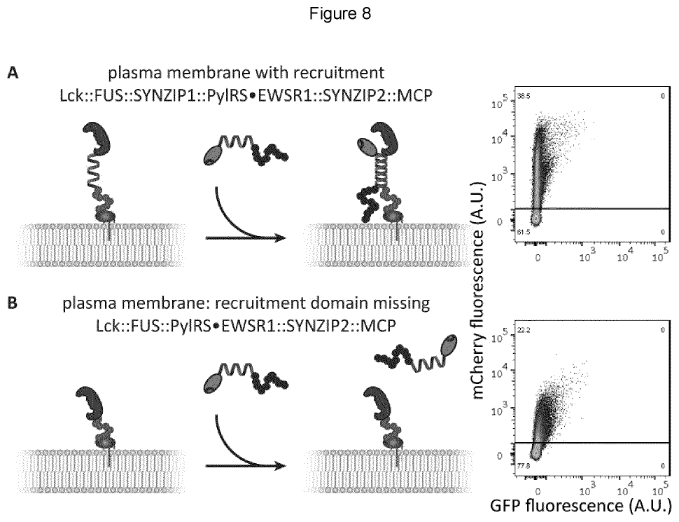

LcK is a cell membrane-targeting signal (Resh, Bba-Mol Cell Res 1999, 1451:1-16) that adds an amphipathic helix post translationally to the POI. For these experiments, the AFPs LcK::FUS::PylRS and LcK::EWSR1::MCP were co-expressed in HE293T cells (see FIGS. 3 and 6C). Testing of this system with the same dual reporter resulted in a dramatic shift in the signal and a strong selectivity for the expression solely of the MS2-tagged mCherry compared to the control PylRS. See FIG. 4 and FIG. 5 showing a 26-fold selectivity gain as compared to the control. IF and FISH for MCP, PylRS and tRNA show a clear membrane signal with appearance of occasional droplet-like structures and a perfect co-localization of all the components.

Without wishing to be bound by theory, it is assumed that targeting the OT system to a membrane results in a confinement of the components to a 2D surface (i.e. a film), offering an even higher spatial segregation than a cytoplasmic droplet. In accordance with such a cumulative effect of the two combined assembler strategies (LcK for membrane targeting, and FUS/EWSR1 for droplet generation), it was shown that the presence of the FUS/EWSR1 “assemblers” is not a requirement in an LcK-fused (and thus membrane-anchored system) for obtaining selective Amber suppression (data not shown). Nevertheless, the combination of the LcK-targeting with FUS/EWSR1 resulted in a higher selectivity of the system. Further, it was found that swapping the MS2-tag on the fluorescent reporters, yielded a swapped selectivity in the FFC data, underlining the selective (orthogonal) translation of the MS2-tagged mRNA.

For a further LcK based experiment, the AFP constructs LcK::FUS::SYNZIP1::PylRS and EWSR1::SYNZIP2::MCP; were co-expressed in HE293T cells (see FIG. 8A). Testing of this system with the same dual reporter resulted in a dramatic shift in the signal and a strong selectivity for the expression solely of the MS2-tagged mCherry. Upon expression SYNZIP1 and 2 pair and recruit MCP to a plasma membrane based OT organelle. In a comparative approach co-expressing the AFP constructs LcK::FUS::PylRS and EWSR1::SYNZIP2::MCP, wherein SYNZIP1 is missing, no selectivity of translation could be observed (see FIG. 8B) EB1 is a microtubule plus ends-targeting signal ((Nehlig A, Molina A, Rodrigues-Ferreira S, Honoré S, Nahmias C. Regulation of end-binding protein EB1 in the control of microtubule dynamics. Cell Mol Life Sci. 2017; 74(13):2381-2393. doi:10.1007/s00018-017-2476-2). For these experiments, the AFP construct EB1::PylRS with EB1::MCP, EB1:FUS::PylRS with EB1::EWSR1::MCP or EB1::FUS::MCP::PylRS were expressed in HE293T cells. Testing of this system with the same dual reporter resulted in a shift in the signal and a strong selectivity for the expression solely of the MS2-tagged mCherry compared to the control PylRS. See FIG. 6B.

CG1 is a nuclear membrane-targeting signal (Kim S J, Fernandez-Martinez J, Nudelman I, et al. Integrative structure and functional anatomy of a nuclear pore complex. Nature. 2018; 555(7697):475-482. doi:10.1038/nature26003) For these experiments, the AFP constructs CG1::FUS::PylRS and CG1::EWSR1::MCP were co-expressed in HE293T cells.

Testing of this system with the same dual reporter resulted in a shift in the signal and a strong selectivity for the expression solely of the MS2-tagged mCherry compared to the control PylRS. See FIG. 6E.

EBAG9_{full length} and EBAG9₁₋₂₉ are Golgi membrane-targeting signals (Engelsberg A, Hermosilla R, Karsten U, Schulein R, Dörken B, Rehm A. The Golgi protein RCAS1 controls cell surface expression of tumor-associated O-linked glycan antigens. J Biol Chem. 2003; 278(25):22998-23007. doi:10.1074/jbc.M301361200). For these experiments, the AFP constructs EBAG9₁₋₂₉::FUS::PylRS and EBAG9₁₋₂₉::EWSR1::MCP were co-expressed in HE293T cells. Testing of this system with the same dual reporter resulted in a shift in the signal and a strong selectivity for the expression solely of the MS2-tagged mCherry compared to the control PylRS. See FIG. 6F (left side).

CMP Sia Tr is a Golgi membrane-targeting signal (Eckhardt M, Gotza B, Gerardy-Schahn R. Membrane topology of the mammalian CMP-sialic acid transporter. J Biol Chem. 1999; 274(13):8779-8787. doi:10.1074/jbc.274.13.8779). For these experiments, the AFP constructs CMP Sia Tr::FUS::PylRS and CMP Sia Tr::MCP were co-expressed in HE293T cells. Testing of this system with the same dual reporter resulted in a shift in the signal and a strong selectivity for the expression solely of the MS2-tagged mCherry compared to the control PylRS. See FIG. 6F (right side).

P450 2C1₁₋₂₇ is an ER membrane-targeting signal (Fazal F M, Han S, Parker K R, et al. Atlas of Subcellular RNA Localization Revealed by APEX-Seq. Cell. 2019; 178(2):473-490.e26. doi:10.1016/j.cell.2019.05.027). For these experiments, the AFP constructs P450 2C1₁₋₂₇::FUS::PylRS and P450 2C1₁₋₂₇::EWSR1::MCP or P450 2C1₁₋₂₉::FUS::MCP::PylRS were co-expressed in HE293T cells. Testing of this system with the same dual reporter resulted in a shift in the signal and a strong selectivity for the expression solely of the MS2-tagged mCherry compared to the control PylRS. See FIG. 6G.

### Example 4—Validation of the Selectivity Gain being Specific to the Interaction of the mRNA MS2-Tag and MCP

To validate that the observed selectivity gain was specific to the interaction of the MCP segment with the MS2-tag of the mRNA, all the OT systems were characterized by expressing the RS assembler fusion of each OT system without MCP. As expected, no selective orthogonal translation of MS2-tagged mRNA was observed in those cases (See FIG. 6 A to G). Additionally, a reporter inversion was performed by moving the MS2-tag from the mCherry to the GFP cassette in the dual-color reporter, which as expected inverted selectivity of the system towards dominant GFP expression (data not shown). This established that the OT systems acted selectively on the MS2-tagged RNA.

### Example 5—Introduction of Multiple ncAAs into the Same POI

GCE can also be used to introduce multiple ncAAs into the same POI (see, e.g., Liu et al., Annu Rev Biochem 2010, 79:413-444; Lemke, ChemBioChem 2014, 15:1691-1694; Chin, Nature 2017, 550; 53-60). However, only very few publications report on more than one, that is, two- or three-codon suppression in the same protein in eukaryotes, as yields typically suffer compared to single-codon suppression (see Xiao et al., Angew Chem 2013, 52:14080-14083; Schmied et al., J Am Chem Soc 2014, 136:15577-15583; Zhang et al., Biochem Biophys Res Co 2017, 489:490-496). Notably, even dual- and triple-Amber proteins were still suppressed with the OT organelle (data not shown).

### Example 6—OT with 3-Iodophenylalanine

To ensure that also other ncAAs can be translated by the OT assembly, another structurally different ncAA (3-iodophenylalanine) was tested which is a phenylalanine derivative instead of a lysine derivative (such as SCO) and is encoded by a different PylRS mutant (N346A, C348A) (see Wang et al., ACS Chem Biol 2013, 8:405-415). Consistent results were also observed for this system (FIG. 2C).

### Example 7—OT with Different Selector Codons

As Opal and Ochre codons are highly abundant in eukaryotic genomes (52% Opal, 28% Ochre in the human genome), the Amber codon is by far the most used for GCE in eukaryotes. In addition, genomic approaches to orthogonal translation by removing those codons in the entire eukaryotic genome would be even more challenging then for the Amber codon and are currently beyond the state of the art. However, in the OT systems of the present invention, a simple mutation in the anticodon loop of the tRNA^{Pyl}, as well as in the respective codon in the MS2-tagged POI-encoding mRNA allows for orthogonal translation of those codons. FFC analysis revealed that the OT systems of the invention provide freedom of choice with respect to the stop (selector) codon (FIG. 2C, E). In fact, Opal suppression turned out to be the best performing system, showing an 11-fold selectivity increase. Ochre suppression still showed fivefold selectivity increase with 20% efficiency.

### Example 8—Orthogonal Translation of Proteins of Various Cellular Compartments

To visualize the power of the OT^{K2::P1} system (the best performing Amber suppression OT system in terms of selectivity and efficiency) beyond “simple” reporters, it was intended to show differential expression of human nucleoporin 153 (Nup153) versus cytoskeletal vimentin. Nup153 locates to the nuclear pore complex and is more than 1500 amino acids long. Hence, its mRNA is approximately six-fold larger than those of the fluorescent protein reporters used above. For this experiment a previously described C-terminal GFP fusion, with an Amber mutation (Nup153::EGFP^{149TAG}) was used that gave rise to a characteristic nuclear envelope stain in confocal images only if Amber suppression was successful (see Nikic et al., Angew Chem 2016, 55:16172-16276). Said Nup153::EGFP^{149TAG} was now tagged at the mRNA level with an MS2-tag (nup153::egfp^{149TAG}::ms2) and co-expressed from the same plasmid with vimentin (a cytoskeletal protein) containing an Amber codon at position 116 fused to mOrange (Vim^{116TAG}::mOrange). Expression in HEK293T cells resulted in production of both proteins in the presence of the cytoplasmic PylRS showing the characteristic nuclear envelope and cytoskeletal staining, respectively. Using the OT^{K2::P1} assembly only Nup153::GFP was visible (selective nuclear rim stain in confocal images of the co-transfected HEK293T cells). Consistent results were also observed in COS-7 cells. Swapping the MS-tag to vimentin inverted the effect, so that only Vim^{116TAG}::mOrange was visible (observed for both COS-7 and HEK293T cell experiments). This showed that the OT^{K2::P1} worked for dramatically different mRNAs.

### Example 9—Orthogonal Translation of Transmembrane Proteins

It was also shown that transmembrane proteins can be selectively expressed using the OT^{K2:P1} assembly. Membrane protein expression represents another layer of translational complexity, as ribosomes need to bind the endoplasmic reticulum during translation, where the proteins are co-translationally inserted into the membrane. In this experiment, a fusion of insulin receptor 1 with an Amber codon at position 676 with mOrange (INSR^{676TAG}::mOrange) was used, which locates to the plasma membrane and gives rise to a characteristic plasma membrane stain in HEK293T cells (see Nikic et al., Angew Chem 2014, 53:2245-2249). This construct was tagged with an MS2-tag in the 3′ UTR and cloned with Nup153::EGFP^{149TAG} into one dual-cassette plasmid. Then the construct was expressed in HEK293T cells either in the presence of the cytoplasmic PylRS system or in the presence of the OT^{K2::P1} assembly. In the presence of the OT^{K2::P1} assembly, selective expression of the MS2-tagged protein and the expected plasma membrane localization of INSR^{676TAG}::mOrange were observed (data not shown), indicating the potential of the OT system of the present invention to participate in even more complex membrane-associated translational processes.

### Example 10—Spatial Distribution of Elements of the OT System in the Cell

The spatial distribution of AFPs and particularly PylRS in cells was assessed using immunofluorescence (IF). Additionally, fluorescence in situ hybridization (FISH) was used for detecting tRNA^{Pyl}. In contrast to the dual color reporter used in the FFC experiments above, in all IF/FISH experiments a single color NLS-GFP^{39TAG} reporter that was fused to an MS2-tag (nls-gfp^{39TAG}::ms2) was used to identify cells active in Amber suppression (this yields a green nucleus if Amber suppression is successful and helped to optimize distinguishable color channels). IF and FISH stainings showed that in contrast to cytoplasmic PylRS, the P1 system formed small, intracellular assembler::PylRS droplets (data not shown). This indicated the occurrence of phase separation. The tRNA^{Pyl} co-localized well with highly dispersed assembler::PylRS droplets, indicating that it could nicely partition into the assembler::PylRS phase. Additional stainings showed further co-localization with assembler::MCP (data hot shown). Compared to P1, the P2 system showed larger but still multiple dispersed droplet-like structures (data not shown). With the combination of both assembler strategies (K1::P1, K2::P1, K1::P2, K2::P2) the formation of large micron-sized organelle-like structures in the cytoplasm was observed, these structures were in most cases localized to few or even a single position per cell. For the combined assemblers, mRNA::ms2, tRNA^{Pyl}, assembler::PylRS and assembler::MCP all co-localized to organelle-like structures. The combination of the two assembler strategies, that is, phase separation paired with spatial targeting by kinesin truncations, yielded the best confinement as determined by FISH and IF and the highest selectivity increase. This is consistent with the hypothesis that the higher spatial segregation and thus higher local concentration of the tRNA^{Pyl}, PylRS and mRNA correlates with higher selectivity.

Ribosomes were stained to see whether they co-localize to the OT^{K2::P1} assembly. IF staining of the ribosomal protein RPL26L1 revealed strong co-localization with the OT^{K2::P1} organelle (data not shown) demonstrating ribosome recruitment, tentatively due to binding to mRNA::ms2 during translation. High ribosomal mobility can also explain why it was possible to successfully express the membrane protein INSR (construct: INSR^{676TAG}::mOrange::ms2). Without wishing to be bound by theory, the experimental results strongly suggests that selective orthogonal translation happens within close proximity of, potentially even inside, the OT assemblies, by a set of recruited ribosomes that are near or fully immersed into a concentrated pool of tRNA^{Pyl}. tRNA^{Pyl} itself is recruited to the OT^{K2::P1} assembly due to its affinity for assembler::PylRS and can readily co-partition into the droplet to be aminoacylated with its cognate ncAA, while assembler::MCP recruits MS2-tagged mRNA. This in turn attracts ribosomes to co-partition into the dense phase formed by the dual assembler system (K2::P1=KIF16B::FUS::PylRS and KIF16B::EWSR1::MCP), which maintains access to other translation factors for translation to function. Ribosomes elsewhere in the cytoplasm that are not exposed to tRNA^{Pyl} perform their canonical function to terminate translation whenever they encounter a stop codon.

### Example 11—Further OT Systems

In addition to the OT systems described in the preceding examples, a variety of other OT systems were tested and found to allow for selective orthogonal translation of the reporter (i.e. the POI). A summary of these experiments is shown in the Table 1 below. Unless noted otherwise, the cytoplasmic NES-PylRS system as previously described by Nikic et al. (Angew Chem Int Ed Engl 2016, 55(52):16172-16176) but with the corresponding AF, AA or AAAF mutations was used as a nonspecific reference (negative control). All experiments were performed in presence of the codon-specific tRNA^{Pyl} and PylRS mutant corresponding ncAAs.

TABLE 1
Tested OT systems

| Fusion protein(s) | | |
|---|---|---|
| comprising O-RS and | | |
| RNA-TP segments | Reporter (POI) | cell lines |
| EWSR1-MCP•FUS-PylRS^{AF} | GFP^{39TAG}-2xMS2•mCherry^{185TAG} | HEK293T/COS-7 |
| | mCherry^{185TAG}-2xMS2•GFP^{39TAG} | |
| FUS-MCP•FUS-PylRS^{AF} | mCherry^{185TAG}-2xMS2•GFP^{39TAG} | HEK293T |
| FUS-MCP-PylRS^{AF} | GFP^{39TAG}-2xMS2•mCherry^{185TAG} | HEK293T/COS-7 |
| | mCherry^{185TAG}-2xMS2•GFP^{39TAG} | |
| MCP-PylRS^{AF} | GFP^{39TAG}-2xMS2•mCherry^{185TAG} | HEK293T/COS-7 |
| | mCherry^{185TAG}-2xMS2•GFP^{39TAG} | |
| SPD5-MCP•SPD5-PylRS^{AF} | GFP^{39TAG}-2xMS2•mCherry^{185TAG} | HEK293T |
| | mCherry^{185TAG}-2xMS2•GFP^{39TAG} | |
| SPD5-MCP-PylRS^{AF} | GFP^{39TAG}-2xMS2•mCherry^{185TAG} | HEK293T |
| | mCherry^{185TAG}-2xMS2•GFP^{39TAG} | |
| KIF16B-PylRS^{AF}•KIF16B-MCP | GFP^{39TAG}-2xMS2•mCherry^{185TAG} | HEK293T/COS-7 |
| | mCherry^{185TAG}-2xMS2•GFP^{39TAG} | |
| KIF16B-MCP-PylRS^{AF} | GFP^{39TAG}-2xMS2•mCherry^{185TAG} | HEK293T/COS-7 |
| | mCherry^{185TAG}-2xMS2•GFP^{39TAG} | |
| KIF16B-FUS-PylRS^{AF}•KIF16B-EWSR1-MCP | GFP^{39TAG}-2xMS2•mCherry^{185TAG} | HEK293T/COS-7 |
| | mCherry^{185TAG}-2xMS2•GFP^{39TAG} | |
| | mCherry^{185TAA}-2xMS2•GFP^{39TAA} | |
| | mCherry^{185TGA}-2xMS2•GFP^{39TGA} | |
| | Nup153-EGFP^{149TAG}-2xMS2•Vimentin^{116TAG}-mOrange | |
| | Vimentin^{116TAG}-mOrange-2xMS2•Nup153-EGFP^{149TAG} | |
| | INSR^{676TAG}-EGFP-2xMS2•Nup153-EGFP^{149TAG} | |
| | INSR^{676TAG}-mOrange-2xMS2•Nup153-EGFP^{149TAG} | |
| | INSR^{676TAG}-I NSR-2xMS2•Nup153-EGFP^{149TAG} | |
| KIF16B-SPD5-PylRS^{AF}•KIF16B-SPD5-MCP | GFP^{39TAG}-2xMS2•mCherry^{185TAG} | HEK293T |
| | mCherry^{185TAG}-2xMS2•GFP^{39TAG} | |
| KIF16B-FUS-PylRS^{AA}•KIF16B-EWSR1-MCP | mCherry^{185TAG}-2xMS2•GFP^{39TAG} | HEK293T |
| KIF16B-FUS-PylRS^{AAAF}•KIF16B-EWSR1-MCP | mCherry^{185TAG}-2xMS2•GFP^{39TAG} | HEK293T |
| KIF13A-PylRS^{AF}•KIF13A-MCP | mCherry^{185TAG}-2xMS2•GFP^{39TAG} | HEK293T |
| KIF13A-FUS-PylRS^{AF}•KIF13A-EWSR1-MCP | mCherry^{185TAG}-2xMS2•GFP^{39TAG} | HEK293T |
| | GFP^{39TAG}-2xMS2•mCherry^{185TAG} | |
| | mCherry^{185TAG}-4xBoxB•GFP^{39TAG}-2xMS2 | |
| | mCherry^{185TAA}-2xMS2•GFP^{39TAA} | |
| | mCherry^{185TGA}-2xMS2•GFP^{39TGA} | |
| KIF13A-SPD5-PylRS^{AF}•KIF13A-SPD5-MCP | mCherry^{185TAG}-2xMS2•GFP^{39TAG} | HEK293T |
| KIF13A-FUS-PylRS^{AA}•KIF13A-EWSR1-MCP | mCherry^{185TAG}-2xMS2•GFP^{39TAG} | HEK293T |
| KI F13A-FUS-PylRS^{AAAF}•KIF13A-EWSR1-MCP | mCherry^{185TAG}-2xMS2•GFP^{39TAG} | HEK293T |
| LCK-FUS-PylRS^{AF}•LCK-EWSR1-MCP | GFP^{39TAG}-2xMS2•mCherry^{185TAG} | HEK293T/COS-7 |
| | mCherry^{185TAG}-2xMS2•GFP^{39TAG} | |
| | mCherry^{185TAG}-4xBoxB•GFP^{39TAG}-2xMS2 | |
| | mCherry^{185TAA}-2xMS2•GFP^{39TAA} | |
| | mCherry^{185TGA}-2xMS2•GFP^{39TGA} | |
| | Nup153-EGFP^{149TAG}-2xMS2•Vimentin^{116TAG}-mOrange | |
| | Vimentin^{116TAG}-mOrange-2xMS2•Nup153-EGFP^{149TAG} | |
| LCK-FUS-MCP-PylRS^{AF} | mCherry^{185TAG}-2xMS2•GFP^{39TAG} | HEK293T |
| | mCherry^{185TAG}-4xBoxB•GFP^{39TAG}-2xMS2 | |
| LCK-FUS-PylRS^{AAAF}•LCK-EWSR1-MCP | mCherry^{185TAG}-2xMS2•GFP^{39TAG} | HEK293T |
| | mCherry^{185TAG}-4xBoxB•GFP^{39TAG}-2xMS2 | |
| LCK-FUS-PylRS^{AF}•LCK-EWSR1-4xλ_{N22} | mCherry^{185TAG}-4xBoxB•GFP^{39TAG} | HEK293T |
| | mCherry^{185TAG}-4xBoxB•GFP^{39TAG}-2xMS2 | |
| LCK-PylRS^{AF}•LCK-MCP | GFP^{39TAG}-2xMS2•mCherry^{185TAG} | HEK293T/COS-7 |
| | mCherry^{185TAG}-2xMS2•GFP^{39TAG} | |
| | mCherry^{185TAG}-4xBoxB•GFP^{39TAG}-2xMS2 | |
| LCK-PylRS^{AAAF}•LCK-MCP | mCherry^{185TAG}-2xMS2•GFP^{39TAG} | HEK293T |
| | mCherry^{185TAG}-4xBoxB•GFP^{39TAG}-2xMS2 | |
| LCK-FUS-3xMCP-PylRS^{AF} | mCherry^{185TAG}-2xMS2•GFP^{39TAG} | HEK293T |
| | mCherry^{185TAG}-4xBoxB•GFP^{39TAG}-2xMS2 | |
| TOM20-FUS-PylRS^{AF}•TOM20-EWSR1-MCP | mCherry^{185TAG}-2xMS2•GFP^{39TAG} | HEK293T |
| | mCherry^{185TAG}-4xBoxB•GFP^{39TAG}-2xMS2 | |
| TOM20-FUS-PylRS^{AF}•TOM20-EWSR1-4xλ_{N22} | mCherry^{185TAG}-4xBoxB•GFP^{39TAG} | HEK293T/COS-7 |
| | mCherry^{185TAG}-4xBoxB•GFP^{39TAG}-2xMS2 | |
| TOM20-FUS-PylRS^{AA}•TOM20-EWSR1-MCP | mCherry^{185TAG}-2xMS2•GFP^{39TAG} | HEK293T |
| TOM20-FUS-PylRS^{AA}•TOM20-EWSR1-4xλ_{N22} | mCherry^{185TAG}-4xBoxB•GFP^{39TAG} | HEK293T |
| TOM20-FUS-PylRS^{AAAF}•TOM20-EWSR1-MCP | mCherry^{185TAG}-2xMS2•GFP^{39TAG} | HEK293T |
| TOM20-FUS-PylRS^{AAAF}•TOM20-EWSR1-4xλ_{N22} | mCherry^{185TAG}-4xBoxB•GFP^{39TAG} | HEK293T |
| | mCherry^{185TAG}-4xBoxB•GFP^{39TAG}-2xMS2 | |
| TOM20-3xMCP-PylRS^{AF} | mCherry^{185TAG}-2xMS2•GFP^{39TAG} | HEK293T |
| | mCherry^{185TAG}-4xBoxB•GFP^{39TAG}-2xMS2 | |
| TOM20-FUS-3xMCP-PylRS^{AF} | mCherry^{185TAG}-2xMS2•GFP^{39TAG} | HEK293T |
| TOM20-FUS-3xMCP-PylRS^{AAAF} | mCherry^{185TAG}-2xMS2•GFP^{39TAG} | HEK293T |
| | mCherry^{185TAG}-4xBoxB•GFP^{39TAG}-2xMS2 | |
| TOM20-FUS-4xλ_{N22}-PylRS^{AF} | mCherry^{185TAG}-4xBoxB•GFP^{39TAG}-2xMS2 | HEK293T/COS-7 |
| TOM20-FUS-4xλ_{N22}-PylRS^{AAAF} | mCherry^{185TAG}-4xBoxB•GFP^{39TAG}-2xMS2 | HEK293T |
| KIF16B-FUS-4xλ_{N22}-PylRS^{AF} | mCherry^{185TAG}-4xBoxB•GFP^{39TAG} | HEK293T |
| | mCherry^{185TAG}-4xBoxB•GFP^{39TAG}-2xMS2 | |
| KIF16B-SPD5-4xλ_{N22}-PylRS^{AF} | mCherry^{185TAG}-4xBoxB•GFP^{39TAG} | HEK293T |
| KIF16B-SPD5-MCP-PylRS^{AF} | mCherry^{185TAG}-2xMS2•GFP^{39TAG} | HEK293T |
| FRB-CD28-FUS-PylRS^{AA}•FRB-CD28-EWSR1-MCP | mCherry^{185TAG}-2xMS2•GFP^{39TAG} | HEK293T |
| FRB-CD28-FUS-PylRS^{AA}•FRB-CD28-EWSR1-4xλ_{N22} | mCherry^{185TAG}-4xBoxB•GFP^{39TAG} | HEK293T |
| FRB-CD28-FUS-PylRS^{AF}•FRB-CD28-EWSR1-MCP | mCherry^{185TAG}-2xMS2•GFP^{39TAG} | HEK293T |
| FRB-CD28-FUS-PylRS^{AF}•FRB-CD28-EWSR1-4xλ_{N22} | mCherry^{185TAG}-4xBoxB•GFP^{39TAG} | HEK293T |
| FUS-CD28-FUS-PylRS^{AA}•FUS-CD28-EWSR1-MCP | mCherry^{185TAG}-2xMS2•GFP^{39TAG} | HEK293T |
| | mCherry^{185TAG}-4xBoxB•GFP^{39TAG}-2xMS2 | |
| FUS-CD28-FUS-PylRS^{AA}•FUS-CD28-EWSR1-4xλ_{N22} | mCherry^{185TAG}-4xBoxB•GFP^{39TAG} | HEK293T |
| | mCherry^{185TAG}-4xBoxB•GFP^{39TAG}-2xMS2 | |
| FUS-CD28-FUS-PylRS^{AF}•FUS-CD28-EWSR1-MCP | mCherry^{185TAG}-2xMS2•GFP^{39TAG} | HEK293T |
| FUS-CD28-FUS-PylRS^{AF}•FUS-CD28-EWSR1-4xλ_{N22} | mCherry^{185TAG}-4xBoxB•GFP^{39TAG} | HEK293T |
| FRB-CD28-FUS-MCP-PylRS^{AA} | mCherry^{185TAG}-2xMS2•GFP^{39TAG} | HEK293T |
| | mCherry^{185TAG}-4xBoxB•GFP^{39TAG}-2xMS2 | |
| FRB-CD28-FUS-MCP-PylRS^{AF} | mCherry^{185TAG}-2xMS2•GFP^{39TAG} | HEK293T |
| | mCherry^{185TAG}-4xBoxB•GFP^{39TAG}-2xMS2 | |
| FUS-CD28-FUS-MCP-PylRS^{AF} | mCherry^{185TAG}-2xMS2•GFP^{39TAG} | HEK293T |
| | mCherry^{185TAG}-4xBoxB•GFP^{39TAG}-2xMS2 | |
| FUS-CD28-FUS-MCP-PylRS^{AA} | mCherry^{185TAG}-2xMS2•GFP^{39TAG} | HEK293T |
| KIF16B-FUS-MCP-PylRS^{AF} | mCherry^{185TAG}-2xMS2•GFP^{39TAG} | HEK293T/COS-7 |
| | mCherry^{185TAG}-4xBoxB•GFP^{39TAG}-2xMS2 | |
| | GFP^{39TAG}-2xMS2•mCherry^{185TAG} | |
| KIF13A-FUS-MCP-PylRS^{AF} | mCherry^{185TAG}-2xMS2•GFP^{39TAG} | HEK293T |
| TOM20-FUS-V5-PylRS^{AF}•TOM20-EWSR1-HA-MCP | mCherry^{185TAG}-2xMS2•GFP^{39TAG} | HEK293T |
| TOM20-FUS-V5-PylRS^{AF}•TOM20-EWSR1-Myc-4xλ_{N22} | mCherry^{185TAG}-4xBoxB•GFP^{39TAG} | HEK293T |
| TOM20-FUS-V5-PylRS^{AA}•TOM20-EWSR1-HA-MCP | mCherry^{185TAG}-2xMS2•GFP^{39TAG} | HEK293T |
| TOM20-FUS-V5-PylRS^{AA}•TOM20-EWSR1-Myc-4xλ_{N22} | mCherry^{185TAG}-4xBoxB•GFP^{39TAG} | HEK293T |
| TOM20-FUS-V5-PylRS^{AAAF}•TOM20-EWSR1-HA-MCP | mCherry^{185TAG}-2xMS2•GFP^{39TAG} | HEK293T |
| TOM20-FUS-V5-PylRS^{AAAF}•TOM20-EWSR1-Myc-4xλ_{N22} | mCherry^{185TAG}-4xBoxB•GFP^{39TAG} | HEK293T |
| KIF16B-VSV-G-FUS-PylRS^{AF}•KIF16B-EWSR1-MCP | mCherry^{185TAG}-2xMS2•GFP^{39TAG} | HEK293T |
| KIF16B-VSVG-FUS-PylRS^{AF}•KIF16B- | mCherry^{185TAG}-2xpp7•GFP^{39TAG} | HEK293T |
| EWSR1-2xPCP | | |
| KIF16B-VSVG-FUS-PylRS^{AF}•KIF16B- | mCherry^{185TAG}-4xpp7•GFP^{39TAG} | HEK293T |
| EWSR1-2xPCP | | |
| KIF16B-VSVG-FUS-PylRS^{AF}•KIF16B- | mCherry^{185TAG}-6xpp7•GFP^{39TAG} | HEK293T |
| EWSR1-2xPCP | | |

### Example 12—Further OT Systems

In addition to the OT systems described in the preceding examples, a variety of similar OT systems were tested, differing with respect to the mRNA targeting components, and were found to allow for selective orthogonal translation of the reporter (i.e. the POI). A summary of these experiments is shown in the Table 2 below. The results are shown in FIGS. 7A, B and C. The cytoplasmic NES-PylRS system as previously described by Nikic et al. (Angew Chem Int Ed Engl 2016, 55(52):16172-16176) was used as a nonspecific reference (negative control). All experiments were performed in presence of the codon-specific tRNA^{Pyl} and PylRS mutant corresponding ncAAs.

TABLE 2
Tested OT systems

| Fusion protein(s) | | |
|---|---|---|
| comprising O-RS and | | cell |
| RNA-TP segments | Reporter (POI) | lines |
| EBAG9₁₋₂₉::FUS::PylRS•EBAG9₁₋₂₉:: | mCherry^{190TAG}- | HEK293T |
| EWSR1::4xλ_{N22} | 4xBoxB•GFP^{39TAG} | |
| EBAG9₁₋₂₉::FUS::PylRS• EBAG9₁₋₂₉:: | mCherry^{190TAG}- | HEK293T |
| EWSR1::MCP | 2xMS2•GFP^{39TAG} | |
| EBAG9₁₋₂₉::FUS::PylRS • EBAG9₁₋₂₉:: | mCherry^{190TAG}- | HEK293T |
| EWSR1::2xPCP | 2xpp7•GFP^{39TAG} | |

The results are shown in FIGS. 7A, B and C.

### Example 13—Further OT Fusion Constructs Tested

In addition to the OT systems described in the preceding examples, a variety of other OT fusion constructs were prepared and tested and found to allow for selective orthogonal translation of the reporter (i.e. the POI). A summary of the tested constructs is shown in the Table 3 below. Unless noted otherwise, the cytoplasmic NES-PylRS system as previously described by Nikic et al. (Angew Chem Int Ed Engl 2016, 55(52):16172-16176) but with the corresponding AF, AA or AAAF mutations, or one of the Pyl RS mutants CpkRS, CbzRS, IFRS1 und OMeRS was used as a nonspecific reference (negative control).

All experiments were performed in the presence of the codon specific tRNA^{Pyl} and PylRS mutant corresponding noncanoncial amino acids [for example CpkRS with cyclopropene-L-Lysine, CbzRS with N(epsilon)-Benzyloxycarbonyl-L-lysine, IFRS-1 with 3-Iodo-L-phenylalanine, OMeRS with 4-Methoxy-L-phenylalanine)].

All constructs were tested with a respective reporter ms2-loops for MCP, boxB-loops for λ_{N22}, pp7-loops for PCP.

In all fusion constructs the synthetases should be freely interchangeable.

For the SYNZIP constructs it is important to note that SYNZIP1 forms a pair with SYNZIP2 and SYNZIP3 forms a pair with SYNZIP4. In principle all other described SYNZIPs should work similarly (pubs.acs.org/doi/pdf/10.1021/ja907617a).

TABLE 3
Tested OT fusion constructs

| | AA |
|---|---|
| | SEQ |
| Fusion proteins comprising | ID |
| O-RS or RNA-TP segments | NO: |
| EBAG9₁₋₂₉::FUS::PylRS(AF) | 320 |
| EBAG9::PylRS(AF) | 322 |
| EBAG9::FUS::PylRS(AF) | 324 |
| EBAG9::MCP | 326 |
| EBAG9::EWSR1::MCP | 328 |
| EBAG9₁₋₂₉::PylRS(AF) | 330 |
| EBAG9₁₋₂₉::FUS::PylRS(AF) | 332 |
| EBAG9₁₋₂₉::MCP | 334 |
| EBAG9₁₋₂₉::EWSR1::MCP | 336 |
| EBAG9::EWSR1::4xλ_{N22} | 338 |
| EBAG9₁₋₂₉::EWSR1::4xλ_{N22} | 340 |
| EBAG9::PylRS(AA) | 342 |
| EBAG9::PylRS(AAAF) | 344 |
| EBAG9::FUS::PylRS(AA) | 346 |
| EBAG9::FUS::PylRS(AAAF) | 348 |
| EBAG9₁₋₂₉::FUS::PylRS(AA) | 350 |
| EBAG9₁₋₂₉::FUS::PylRS(AAAF) | 352 |
| EBAG9₁₋₂₉::FUS::MCP::PylRS(AF) | 354 |
| CG1::PylRS(AF) | 356 |
| CG1::PylRS(AA) | 358 |
| CG1::PylRS(AAAF) | 360 |
| CG1::FUS::PylRS(AA) | 362 |
| CG1::FUS::PylRS(AAAF) | 364 |
| CG1::MCP | 366 |
| CG1::EWSR1::MCP | 368 |
| CG1::FUS::PylRS(AF) | 370 |
| CG1::FUS::MCP::PylRS(AF) | 372 |
| CMP-SaTr::PylRS(AF) | 374 |
| CMP-SaTr::PylRS(AA) | 376 |
| CMP-SaTr::PylRS(AAAF) | 378 |
| CMP-SaTr::FUS::PylRS(AA) | 380 |
| CMP-SaTr::FUS::PylRS(AAAF) | 382 |
| CMP-SaTr::MCP | 384 |
| CMP-SaTr::EWSR1::MCP | 386 |
| CMP-SaTr::PylRS(AF)EWSR1::4xλ_{N22} | 388 |
| CMP-SaTr::FUS::PylRS(AF) | 390 |
| P450 2C1₁₋₂₇::PylRS(AF) | 392 |
| P450 2C1₁₋₂₇::MCP | 394 |
| P450 2C1₁₋₂₇::FUS::PylRS(AF) | 396 |
| P450 2C1₁₋₂₇::EWSR1::MCP | 398 |
| P450 2C1₁₋₂₇::FUS::MCP::PylRS(AF) | 400 |
| P450 2C1₁₋₂₉::FUS::MCP::PylRS(AF) | 402 |
| EB1::PylRS(AF) | 404 |
| EB1::PylRS(AA) | 406 |
| EB1::PylRS(AAAF) | 408 |
| EB1::FUS::PylRS(AA) | 410 |
| EB1::FUS::PylRS(AAAF) | 412 |
| EB1::MCP | 414 |
| EB1::EWSR1::MCP | 416 |
| EB1::EWSR1::4xλ_{N22} | 418 |
| EB1::FUS::PylRS(AF) | 420 |
| EB1::FUS::MCP::PylRS(AF) | 422 |
| TOM20::FUS::PCP::PylRS(AF) | 424 |
| TOM20::FUS::2xPCP::PylRS(AF) | 426 |
| TOM20::FUS::4xλ_{N22}::PylRS(AF) | 428 |
| LCK::FUS::2xPCP::CbzRS | 430 |
| LCK::FUS::PCP::CbzRS | 432 |
| TOM20::FUS::CbzRS | 434 |
| TOM20::FUS::2xPCP::CbzRS | 436 |
| TOM20::FUS::4xλ_{N22}::CbzRS | 438 |
| EBAG9₁₋₂₉::FUS::PCP::PylRS(AF) | 440 |
| EBAG9₁₋₂₉::FUS::4xλ_{N22}::IFRS1 | 442 |
| KIF16B::EWSR1::Myc::2xPCP | 444 |
| KIF16B::EWSR1::HA::2xPCP | 446 |
| EBAG9₁₋₂₉::EWSR1::Myc::2xPCP | 448 |
| EBAG9₁₋₂₉::EWSR1::HA::2xPCP | 450 |
| LCK::CbzRS | 452 |
| LCK::FUS::CbzRS | 454 |
| TOM20::FUS::SYNZIP1::CpkRS | 456 |
| KIF16B::FUS::CbzRS | 458 |
| EBAG9₁₋₂₉::FUS::CpkRS | 460 |
| TOM20::FUS::CbzRS | 462 |
| EBAG9₁₋₂₉::FUS::CbzRS | 464 |
| TOM20::FUS::SYNZIP1::CbzRS | 466 |
| KIF16B::FUS::CpkRS | 468 |
| LCK::FUS::CpkRS | 470 |
| LCK::CpkRS | 472 |
| TOM20::FUS::SYNZIP3::CbzRS | 474 |
| TOM20::FUS::SYNZIP3::CpkRS | 476 |
| TOM20::EWSR1::PylRS(AA)::FUS::PylRS(AA) | 478 |
| LCK::PylRS(AF)::FUS::PylRS(AF) | 480 |
| LCK::FUS::PylRS(AF)::EWSR1::PylRS(AF) | 482 |
| LCK::FUS::PylRS(AF)::FUS::PylRS(AF) | 484 |
| TOM20::FUS::PylRS(AF)::EWSR1::PylRS(AF) | 486 |
| TOM20::FUS::PylRS(AF)::FUS::PylRS(AF) | 488 |
| TOM20::EWSR1::4xλ_{N22}::PylRS(AA)::FUS::PylRS(AA) | 490 |
| LCK::EWSR1::MCP::PylRS(AA)::FUS::PylRS(AA) | 492 |
| LCK::PylRS(AA)::FUS::PylRS(AA) | 494 |
| LCK::PylRS(AF)::EWSR1::PylRS(AF) | 496 |
| TOM20::FUS::MCP::PylRS(AF)::EWSR1::PylRS(AF) | 498 |
| TOM20::FUS::4xλ_{N22}::PylRS(AF)::EWSR1::PylRS(AF) | 500 |
| TOM20::FUS::SYNZIP1::MCP::PylRS(AF)::EWSR1::PylRS(AF) | 502 |
| TOM20::FUS::SYNZIP2::MCP::PylRS(AF)::EWSR1::PylRS(AF) | 504 |
| LCK::FUS::SYNZIP1::MCP::PylRS(AF)::EWSR1::PylRS(AF) | 506 |
| LCK::FUS::SYNZIP2::MCP::PylRS(AF)::EWSR1::PylRS(AF) | 508 |
| LCK::PylRS(AA)::EWSR1::PylRS(AA) | 510 |
| LCK::FUS::PylRS(AA)::EWSR1::PylRS(AA) | 512 |
| TOM20::FUS::PylRS(AA)::EWSR1::PylRS(AA) | 514 |
| TOM20::FUS::MCP::PylRS(AA)::EWSR1::PylRS(AA) | 516 |
| TOM20::FUS::4xλ_{N22}::PylRS(AA)::EWSR1::PylRS(AA) | 518 |
| LCK::EWSR1::MCP::PylRS(AF)::FUS::PylRS(AF) | 520 |
| TOM20::EWSR1::4xλ_{N22}::PylRS(AF)::FUS::PylRS(AF) | 522 |
| TOM20::EWSR1::MCP::PylRS(AF)::FUS::PylRS(AF) | 524 |
| LCK::PylRS(AA)::FUS::PylRS(AA) | 526 |
| EBAG9₁₋₂₉::EWSR1::SYNZIP4::4xλ_{N22} | 528 |
| KIF16B::FUS::SYNZIP1::PylRS(AF) | 530 |
| KIF16B::FUS::SYNZIP!::PylRS(AA) | 532 |
| EBAG9₁₋₂₉::EWSR1::SYNZIP2::MCP | 534 |
| TOM20::EWSR1::SYNZIP2::MCP | 536 |
| TOM20::FUS::SYNZIP4::4xλ_{N22}::PylRS(AA) | 538 |
| KIF16B::EWSR1::SYNZIP4::4xλ_{N22} | 540 |
| TOM20::EWSR1::SYNZP4::4xλ_{N22} | 542 |
| TOM20::FUS::SYNZIP1::PylRS(AF) | 544 |
| TOM20::FUS::SYNZIP::3::PylRS(AF) | 546 |
| EBAG9₁₋₂₉::FUS::SYNZIP1::PylRS(AF) | 548 |
| EBAG9₁₋₂₉::FUS::SYNZIP3::PylRS(AF) | 550 |
| TOM20::FUS::SYNZIP1::PylRS(AA) | 552 |
| TOM20::FUS::SYNZIP3::PylRS(AA) | 554 |
| TOM20::FUS::SYNZIP3::PylRS(AAAF) | 556 |
| LCK::FUS::SYNZIP3::PylRS(AF) | 558 |
| LCK::SYNZIP1::PylRS(AF) | 560 |
| LCK::SYNZIP3::PylRS(AF) | 562 |
| SYNZIP2::MCP | 564 |
| LCK::EWSR1::SYNZIP2::MCP | 566 |
| LCK::EWSR1::SYNZIP4::4xλ_{N22} | 568 |
| LCK::SYNZIP2::MCP | 570 |
| EWSR1::SYNZIP2::MCP | 572 |
| LCK::FUS::SYNZIP1::PylRS(AF) | 574 |
| LCK::FUS::SYNZIP3::PylRS(AF) | 576 |
| TOM20::EWSR1::SYNZIP4::2xPCP | 578 |
| TOM20::EWSR1::SYNZIP2::2xPCP | 580 |
| KIF16B::EWSR1::SYNZIP2::MCP | 582 |
| LCK::SYNZIP1::PylRS(AF) | 584 |
| LCK::FUS::SYNZIP1::PylRS(AF) | 586 |
| SYNZIP4::4xλN22 | 588 |
| TOM20::FUS::SYNZIP1::MCP::PylRS(AF) | 590 |
| TOM20::FUS::SYNZIP2::MCP::PylRS(AF) | 592 |
| TOM20::FUS::SYNZIP1::MCP::PylRS(AA) | 594 |
| TOM20::FUS::SYNZIP2::MCP::PylRS(AA) | 596 |
| TOM20::FUS::SYNZIP::MCP::IFRS1 | 598 |
| TOM20::FUS::SYNZIP2::MCP::IFRS1 | 600 |
| TOM20::FUS::SYNZIP3::4xλ_{N22}::CbzRS | 602 |
| EBAG9₁₋₂₉::FUS::SYNZIP3::PCP::PylRS(AF) | 604 |
| EBAG9₁₋₂₉::FUS::SYNZIP4::PylRS(AF) | 606 |
| EBAG9₁₋₂₉::FUS::SYNZIP3::4xλ_{N22}::IFRS1 | 608 |
| LCK::FUS::SYNZIP1::MCP::PylRS(AF) | 610 |
| LCK::FUS::SYNZIP2::MCP::PylRS(AF) | 612 |
| CG1::FUS::SYNZIP1::MCP::PylRS(AF) | 614 |
| CG1::FUS::SYNZIP2::MCP::PylRS(AF) | 616 |
| TOM20::FUS::SYNZIP4::λ_{N22}::CbzRS | 618 |
| EBAG9₁₋₂₉::FUS::SYNZIP4::4xλ_{N22}::IFRS1 | 620 |
| TOM20::FUS::SYNZIP3::4xλ_{N22}::PylRS(AA) | 622 |
| LCK::FUS::SYNZIP1::MCP::PylRS(AA) | 624 |
| TOM20::EWSR1::SYNZIP4::4xλ_{N22}::SYNZIP4::PylRS(AF) | 626 |
| TOM20::FUS::SYNZIP1::MCP::SYNZIP1::PylRS(AF) | 628 |
| TOM20::EWSR1::SYNZIP4::4xλ_{N22}::SYNZIP4::PylRS(AA) | 630 |
| TOM20::FUS::SYNZIP3::4xλ_{N22}::SYNZIP3::PylRS(AA) | 632 |
| LCK::EWSR1::SYNZIP4::4xλ_{N22}::SYNZIP4::PylRS(AF) | 634 |
| LCK::FUS::SYNZIP3::4xλ_{N22}::SYNZIP3::PylRS(AF) | 636 |
| TOM20::FUS::SYNZIP3::4xλ_{N22}::SYNZIP3::PylRS(AF) | 638 |
| TOM20::EWSR1::SYNZIP2::MCP::SYNZIP2::PylRS(AF) | 640 |
| LCK::OMeRS | 642 |
| TOM20::FUS::OMeRS | 644 |
| KIF16B::FUS::OMeRS | 646 |
| LCK::FUS::OMeRS | 648 |
| EBAG9₁₋₂₉:FUS::OMeRS | 650 |
| TOM20::FUS::SYNZIP1::OMeRS | 652 |
| TOM20::FUS::SYNZIP3::OMeRS | 654 |
| SLP3::FUS::PylRS(AF) | 656 |
| SLP3::MCP | 658 |
| SLP3::EWSR1::MCP | 660 |
| SLP3::EWSR1::4xλ_{N22} | 662 |
| SLP3::PylRS(AF) | 664 |
| TOM20::FUS::MCP::PylRS(AF) | 666 |
| KIF16B::1xLAF-1::PylRS(AF) | 668 |
| KIF16B::1xLAF-1::MCP | 670 |
| KIF16B::1xLAF-1::2xPCP | 672 |
| KIF16B::2xLAF-1::2xPCP | 674 |
| KIF16B::2xLAF-1::PylRS(AF) | 676 |
| KIF16B::2xLAF-1::MCP | 678 |

AA: amino acid sequence

### Abbreviations

“-” or Symbols representing a peptidic linkage
“⋅” Symbol representing a combination of polypeptides
AP polypeptide segment acting as assembler
AFP assembler fusion protein
BSA bovine serum albumin
BoxB lambda phase RNA stem-loop, specific binding site of λ_{N22}
CbzRS Methanosarcina mazei PylRS (Y306M, L309G, C348T)
CDS (en-)coding sequence
CG1 CG1 (Nup42) nucleoporin protein for targeting to nuclear membrane
CMPSiaTr CMP sialic acid transporter for targeting to Golgi membrane
CpkRS Methanosarcina mazei PylRS (A302S
EB1 EB1 protein for targeting to microtubule plus ends
EBAG9 Receptor-binding cancer antigen expressed on SiSo cells
EBAG9_{FL} EBAG9 full length protein for targeting to Golgi membrane
EBAG9₁₋₂₉ EBAG9 amino acid residues 1-29 (N-terminal) for targeting to Golgi membrane
EGFP^{149TAG} enhanced green fluorescent protein, amino acid position 149 encoded by Amber codon (TAG)
EP polypeptide segment acting as an effector
ER Endoplasmatic Reticulum
EWSR1 Ewing sarcoma breakpoint region 1 (also termed EWS herein)
FBS fetal bovine serum
FFC fluorescence flow cytometry
FISH fluorescence in situ hybridization
FRB-CD28 synthetic membrane targeting domain derived from transmembrane proteins CD4, FRB (similar to mTOR) and CD28
FSC-A forward scatter area
FUS fused-in sarcoma
FUS-CD28 (synthetic membrane targeting fusion polypeptide derived from CD4, FUS and CD28
GCE genetic code expansion
GFP green fluorescent protein
GFP^{39TAA} green fluorescent protein, amino acid position 39 encoded by Ochre codon (TAA)
GFP^{39TAG} green fluorescent protein, amino acid position 39 encoded by Amber codon (TAG)
GFP^{39TGA} green fluorescent protein, amino acid position 39 encoded by Opal codon (TGA)
GFP^{39,149TAG} green fluorescent protein, each of amino acid positions 39 and 149 encoded by Amber codon (TAG)
GFP^{39,149,182TAG} green fluorescent protein, each of amino acid positions 39, 149 and 182 encoded by Amber codon (TAG)
IC-TP intracellular targeting polypeptide
IDP intrinsically disordered protein
IFRS1 Methanosarcina mazei PylRS (L305M, Y306L, L309S, N346S, C348M)
INSR insulin receptor
INSR^{676TAG} insulin receptor, amino acid position 676 encoded by Amber codon (TAG)
iRFP near-infrared fluorescent protein
KIF13A kinesin family member 13A—Unless specified otherwise herein, “KIF13A” specifically refers to the fragment covering amino acid residues 1-411 of KIF13A wherein P390 is deleted (KIF13A_{1-411,ΔP390}).
KIF16B kinesin family member 16B—Unless specified otherwise herein, “KIF16B” specifically refers to the fragment covering amino acid residues 1-400 of KIF16B (KIF16B₁₋₄₀₀).
λ_{N22} 22 amino acid RNA-binding domain of lambda phage antiterminator protein N
LcK posttranslational modification site for plasma membrane targeting of lymphocyte-specific protein tyrosine kinase
mCherry^{185TAG} mCherry, amino acid position 185 encoded by Amber codon (TAG)
MCP MS2 bacteriophage coat protein
MLC membrane-less compartment
MS2 Enterobacteria phage MS2
MS2-tag two MS2 RNA stem-loops fused to the 3′ untranslated region of the mRNA (or coding sequence therefor)
ms2 MS2-tag
ncAA non-canonical amino acid
NLS nuclear localization sequence
Nup153 nucleoporin 153
O-RS orthogonal aminoacyl tRNA synthetase
OMeRS Methanosarcina mazei PyrRS (A302T, Y384F, N346V, C348W, V401L)
OT assembly spatially enriched components of the GCE machinery in a membrane-less assembly that is able to act as an artificial orthogonally translating (OT) organelle
P450 2C1₁₋₂₇ P450 2C1 residues 1-27 (N-terminal) for targeting of ER membranes
PBS phosphate buffered saline
PCP Bacteriophage coat protein for targeting to pp7 loop tag
PEI polyethylenimine
POI polypeptide of interest (=target polypeptide)
POI^{TAG} POI comprising an Amber-(TAG-)encoded amino acid residue (or coding sequence therefor)
pp7 pp7 loop tag from RNA bacteriophage pp7
PSP phase separation polypeptide
PylRS pyrrolysyl tRNA synthetase
PylRS^{AA} mutant M. mazei pyrrolysyl tRNA synthetase comprising amino acid substitutions N346A and C348A
PylRS^{AF} mutant M. mazei pyrrolysyl tRNA synthetase comprising amino acid substitutions Y306A and Y384F
PylRS^{AAF} mutant M. mazei pyrrolysyl tRNA synthetase comprising amino acid substitutions Y306A, N346A, C348A and Y384F
RNA-TP RNA targeting polypeptide
RS aminoacyl tRNA synthetase
RT room temperature
SCO cyclooctyne lysine
SEM standard error of the mean
SSC saline-sodium citrate (buffer)
SSC-A side scatter area
SSC-W side scatter width
SPD5 spindle-defective protein 5
SYNZIP1 Synthetic coiled coil peptide 1
SYNZIP2 Synthetic coiled coil peptide 2
SYNZIP3 Synthetic coiled coil peptide 3
SYNZIP4 Synthetic coiled coil peptide 4
TOMM20 translocase of outer mitochondrial membrane 20
TOMM20₁₋₇₀ fragment covering amino acid residues 1-70 of TOMM20
tRNA^{Pyl} tRNA that is coupled to pyrrolysyl or another non-canonical amino acid residue by a wild-type or modified PylRS and has an anticodon that, for site-specific incorporation of a (non-canonical) amino acid residue into a POI, is preferably the reverse complement of a selector codon. The tRNA^{Pyl} used in the examples carried the anticodon against the stop codon Amber (tRNA^{Pyl,CUA}), Ochre (tRNA^{Pyl,UUA}) or Opal (tRNA^{Pyl,UCA}) depending on which of these was used as selector codon in the POI-encoding sequence.
3′ UTR 3′ untranslated region
Vim^{116TAG} Vimentin, amino acid position 116 encoded by Amber codon (TAG)

### Sequences

The following sections show the sequences of the polypeptides and polynucleotides described herein.

Nucleic acid sequences are stated in 5′ to 3′ orientation, protein sequences are stated from N- to C-terminus.

Lengthy table referenced here
US20230098002A1-20230330-T00001
Please refer to the end of the specification for access instructions.

LENGTHY TABLES
The patent application contains a lengthy table section. A copy of the table is available in electronic form from the USPTO web site (https://seqdata.uspto.gov/?pageRequest=docDetail&DocID=US20230098002A1). An electronic copy of the table will also be available from the USPTO upon request and payment of the fee set forth in 37 CFR 1.19(b)(3).

1-15. (canceled)

16. An assembler fusion protein (AFP) comprising:
(a) at least one first polypeptide segment acting as assembler (AP) that is selected from:
(a1) a polypeptide segment derived from an intracellular targeting polypeptide (IC-TP segment), wherein said intracellular targeting polypeptide targets, and thus becomes locally enriched at, an intracellular structural element within or directly adjacent to the cytoplasm; and
(a2) a polypeptide segment derived from a phase separation polypeptide (PSP segment), wherein said phase separation polypeptide has the ability to undergo self-association in the cytoplasm of a cell so as to create sites of high local concentration in the cytoplasm, and
(b) at least one second polypeptide segment acting as an effector (EP) that is selected from:
b1) an RNA-targeting polypeptide (RNA-TP) segment, and
b2) an orthogonal aminoacyl tRNA synthetase (O-RS) segment;
wherein said polypeptide segments are functionally linked in said AFP.

17. An assembler fusion protein (AFP) combination comprising at least two AFPs of claim 16.

18. A fusion protein (RNA-TP/O-RS fusion protein) comprising:
(i) at least one RNA-targeting polypeptide (RNA-TP) segment; and
(ii) at least one orthogonal aminoacyl tRNA synthetase (O-RS) segment,
wherein said polypeptide segments are functionally linked in said RNA-TP/O-RS fusion protein.

19. A nucleic acid molecule, or a combination of two or more nucleic acid molecules, comprising:
(i) a nucleotide sequence that encodes at least one AFP of claim 16, or at least one AFP combination of at least two AFPs of claim 16, or
(ii) a nucleic acid sequence complementary to the nucleotide sequence of (i), or
(iii) both of (i) and (ii).

20. A nucleic acid molecule, or a combination of two or more nucleic acid molecules, comprising:
(i) a nucleotide sequence that encodes at least one RNA-TP/O-RS fusion protein of claim 18, or
(ii) a nucleic acid sequence complementary to (i), or
(iii) both of (i) and (ii).

21. An expression cassette comprising the nucleotide sequence of the nucleic acid molecule, or the combination of nucleic acid molecules of claim 19.

22. An expression cassette comprising the nucleotide sequence of the nucleic acid molecule, or the combination of nucleic acid molecules of claim 20.

23. An expression vector comprising at least one expression cassette of claim 21.

24. An expression vector comprising at least one expression cassette of claim 22.

25. A cell comprising at least one nucleic acid molecule, or combination of nucleic acid molecules, of claim 19.

26. A cell comprising at least one nucleic acid molecule, or combination of nucleic acid molecules of claim 20.

27. A method for preparing a polypeptide of interest (POI) comprising in its amino acid sequence one or more non-canonical amino acid (ncAA) residues, wherein the method comprises expressing the POI in a cell in the presence of said one or more ncAAs, wherein the cell comprises:
(i) a POI-encoding nucleotide sequence (CS^{POI}) wherein said one or more ncAA residues of the POI are encoded by selector codon(s),
(ii) a targeting nucleotide sequence (TN) that is functionally linked to the CS^{POI} and is able to interact with an RNA-TP segment of at least one of the AFPs in the cell;
(iii) one or more orthogonal tRNA^{ncAA} (O-tRNA^{ncAA}) molecules which carry the anticodon(s) complementary to the selector codon(s) of the CS^{POI}, and wherein said O-tRNA^{ncAA} molecules together with one or more O-RS segments of at least one of the AFPs in the cell form one or more orthogonal O-RS/O-tRNA^{ncAA} pairs which allow for the introduction of said one or more ncAA residues into the amino acid sequence of the POI;
and wherein the method optionally further comprises recovering the expressed POI.

28. A method for preparing a polypeptide of interest (POI) comprising in its amino acid sequence one or more non-canonical amino acid (ncAA) residues, wherein the method comprises expressing the POI in a cell in the presence of said one or more ncAAs, wherein the cell comprises:
(i) a POI-encoding nucleotide sequence (CS^{POI}) wherein said one or more ncAA residues of the POI are encoded by selector codon(s),
(ii) a targeting nucleotide sequence (TN) that is functionally linked to the CS^{POI} and is able to interact with an RNA-TP segment of at least one of the RNA-TP/O-RS fusion proteins in the cell;
(iii) one or more orthogonal tRNA^{ncAA} (O-tRNA^{ncAA}) molecules which carry the anticodon(s) complementary to the selector codon(s) of the CS^{POI}, and wherein said O-tRNA^{ncAA} molecules together with one or more O-RS segments of the RNA-TP/O-RS fusion proteins in the cell form one or more orthogonal O-RS/O-tRNA^{ncAA} pairs which allow for the introduction of said one or more ncAA residues into the amino acid sequence of the POI;
and wherein the method optionally further comprises recovering the expressed POI.

29. A nucleic acid molecule comprising:
(i) a nucleotide sequence (CSP^{POI}) that encodes a polypeptide of interest (POI), said POI comprising one or more non-canonical amino acid (ncAA) residues which are encoded in the CS^{POI} by selector codons, and
(ii) a targeting nucleotide sequence (TN), wherein an RNA molecule comprising said TN is able to interact via said TN with an RNA-targeting polypeptide (RNA-TP).

30. A kit for preparing a polypeptide of interest (POI) having at least one non-canonical amino acid (ncAA) residue, the kit comprising:
at least one ncAA, or salt thereof, corresponding to the at least one ncAA residue of the POI, and
at least one expression vector of claim 23.

31. A kit for preparing a polypeptide of interest (POI) having at least one non-canonical amino acid (ncAA) residue, the kit comprising:
at least one ncAA, or salt thereof, corresponding to the at least one ncAA residue of the POI, and
at least one expression vector of claim 24.
